(19) United States
(12) Patent Application Publication
(10) Pub. No.: US 2018/0161410 A1
(43) Pub. Date: Jun. 14, 2018

(54) Methods to Treat Diseases with Protein, Peptide, Antigen Modification and Hemopurification

(71) Applicant: Tianxin Wang, Walnut Creek, CA (US)

(72) Inventor: Tianxin Wang, Walnut Creek, CA (US)

(73) Assignee: Tianxin Wang, Walnut Creek, CA (US)

(21) Appl. No.: 15/883,100

(22) Filed: Jan. 30, 2018

Related U.S. Application Data
(62) Division of application No. 15/373,483, filed on Dec. 9, 2016.
Application No. 15/373,483 is a continuation-in-part of application No. 14/246,117, filed on Apr. 6, 2014.

(51) Int. Cl. A61K 39/00; C07K 14/605; C07K 14/58; C07K 14/47; C07K 14/575

(52) U.S. Cl. CPC A61K 39/0008; C07K 14/605; C07K 14/58; A61K 2039/6031; C07K 14/47; C07K 14/575; A61K 39/00

(57) ABSTRACT

The current invention discloses methods to modify protein and peptide and antigen to treat disease such as pathogen infection, autoimmune diseases and cancer. The method involves increasing the molecular weight of the protein by connecting multiple peptide units with site specific conjugation to extend the in vivo half life. The current invention also discloses methods to construct activatable enzyme, which becomes active when they reach the treatment target, therefore provide higher specificity for treatment. The current invention also relates to methods to treat disease with hemopurification.

### CROSS-REFERENCE TO RELATED APPLICATIONS

This application is the divisional application of U.S. patent application Ser. No 15/373,483 filed on Dec. 9, 2016, which claims priority to U.S. Provisional Patent Application 62/265,991 filed on Dec. 11 2015, and U.S. Provisional Patent Application 62/300,924 filed on Feb. 29, 2016. The current application is also a Continuation-In-Part application of U.S. patent application Ser. No. 14/246,117, filed on Apr. 6, 2014, which is a continuation application of U.S. application Ser. No. 13/444,201, filed on Apr. 11, 2012, which claims priority to U.S. Provisional Patent Application No. 61/457,807 filed on Jun. 8, 2011 and U.S. Provisional Patent Application No. 61/516,956 filed on Apr. 12, 2011. The entire disclosure of the prior application is considered to be part of the disclosure of the instant application and is hereby incorporated by reference.

### BACKGROUND OF THE INVENTION

### Field of the Invention

The current invention relates to protein, peptide and antigen modification for pharmaceutical applications and reagents to treat disease such as pathogen infection, auto immune disease and cancer. The method used for protein and peptide modification can extend their half life. The current invention also relates to methods to treat disease with hemopurification.

### Background Information

Protein drugs have changed the face of modern medicine, finding application in a variety of different diseases such as cancer, anemia, and neutropenia. As with any drugs, however, the need and desire for drugs having improved specificity and selectivity for their targets is of great interest, especially in developing second generation of protein drugs having known targets to which they bind. It is also desirable to have a long in vivo half life for the protein drug to reduce their injection frequency to provide a better treatment for patient. Extending the half-life a therapeutic agent, whether being a therapeutic protein, peptide or small molecule, often requires specialized formulations or modifications to the therapeutic agent itself. Conventional modification methods such as pegylation, adding to the therapeutic agent an antibody fragment or an albumin molecule, suffer from a number of profound drawbacks. For example, PEGylated proteins have been observed to cause renal tubular vacuolation in animal models. Renally cleared PEGylated proteins or their metabolites may accumulate in the kidney, causing formation of PEG hydrates that interfere with normal glomerular filtration. Thus, there remains a considerable need for alternative compositions and methods useful for the production of highly pure form of therapeutic agents with extended half-life properties at a reasonable cost.

Extracorporeal therapy is a procedure in which blood is taken from a patient's circulation to have a process applied to it before it is returned to the circulation. All of the apparatus carrying the blood outside the body is termed the extracorporeal circuit. It includes hemodialysis, hemofiltration, plasmapheresis, apheresis and etc. Hemodialysis is a method for extracorporeal removing waste products such as creatinine and urea, as well as free water from the blood when the kidneys are in renal failure. Plasmapheresis is the removal, treatment, and return of (components of) blood plasma from blood circulation. The procedure is used to treat a variety of disorders, including those of the immune system, such as myasthenia gravis, lupus, and thrombotic thrombocytopenic purpura. Hemoperfusion (blood perfusion) is a medical process used to remove toxic or unwanted substances from a patient's blood. Typically, the technique involves passing large volumes of blood over an adsorbent substance. The adsorbent substances most commonly used in hemoperfusion are resins and activated carbon. Hemoperfusion is an extracorporeal form of treatment because the blood is pumped through a device outside the patient's body. Its major uses include removing drugs or poisons from the blood in emergency situations, removing waste products from the blood in patients with renal failure, and as a supportive treatment for patients before and after liver transplantation. Apheresis is a medical technology in which the blood of a donor or patient is passed through an apparatus that separates out one particular constituent and returns the remainder to the circulation. Depending on the substance that is being removed, different processes are employed in apheresis.

### BRIEF DESCRIPTION OF THE DRAWINGS

FIG. 1 shows multivalent homo Fab format with suitable length flexible linker for higher affinity.

FIG. 2 shows hetero Fab format targeting two antigens of the different protein on the cell/microorganism for higher affinity.

FIG. 3 shows Hetero Fab format targeting two epitope sites of the same target protein for higher affinity.

FIG. 4 shows construction of bi-specific antibody and ADC using selective reduction.

FIG. 5 shows bi specific antibody by linking two or more full size antibodies.

FIG. 6 shows an example of the preparation of bi specific antibody by linking two full size antibodies.

FIG. 7 shows uses an example of using immobilized affinity group targeting the carbohydrate on the antibody to selectively protect one FC conjugation site on the antibody to achieve mono conjugation

FIG. 8 shows mono labeling of drug and linker on the antibody.

FIG. 9 shows example of the flexible Ab in mono specific format and bispecific format.

FIG. 10 shows example of the flexible Ab for site specific conjugation for ADC.

FIG. 11 shows example of the flexible bispecific antibody without Fc.

FIG. 12 shows example of the flexible bispecific antibody containing different site specific conjugation residue.

FIG. 13 shows example of alpha-galactosyl-drug conjugate.

FIG. 14 shows an example of alpha-galactosyl-Exenatide conjugate for Exenatide half life extension.

FIG. 15 shows the structure and activating mechanism of self assembly probody

FIG. 16 shows examples of self assembly probody with Fc modifier

FIG. 17 shows the activation mechanism of self assembly probody with Fc modifier

FIG. 18 shows an example of self assembly probody with Fc modifier

FIG. 19 shows example of self assembly probody with heterogenic MM

FIG. 20 shows the structure and activating mechanism of protamer

FIG. 21 shows the structure and activating mechanism of self assembly protamer

FIG. 22 shows examples protamer with half life modifier or drug conjugation

FIG. 23 shows an example of Binding Based Prozyme, which is an enzyme activated upon binding of aptamer

FIG. 24 shows an example of Binding Based Prozyme, which is an enzyme activated upon binding of antibody

FIG. 25 shows the scheme of ABP (antibody binding partner)-linker-EIP (enzyme inhibition partner) based Prozyme

FIG. 26 shows the examples of format of ABP (antibody binding partner)-linker-EIP (enzyme inhibition partner) based prozyme

FIG. 27 shows the scheme of Cleavage Based Prozyme, which is an enzyme activated with second enzyme

FIG. 28 shows sialidase based prozyme and its activation by tumor enzyme

FIG. 29 shows an example of uPA activated sialidase prozyme to treat cancer

FIG. 30 shows example of sialidase-lipid conjugate and sialidase-lipid-folic acid conjugate for cancer treatment.

FIG. 31 shows an examples of a block polymer made of two PEG blocks connected with a biodegradable polylactic acid.

FIG. 32 shows different formats of biodegradable PEG and the biodegradable HGH dimer.

FIG. 33 shows an example of HGH trimer that can extend HGH in vivo half life.

FIG. 34 shows an example of the HGH trimer and its preparation

FIG. 35 shows an example of HGH trimer using 3 arm linker

FIG. 36 shows another example of HGH trimer using 3 arm linker

FIG. 37 shows the scheme of crosslink HGH with affinity group to extend its in vivo half life

FIG. 38 shows the scheme of crosslink HGH with antibody to extend its in vivo half life

FIG. 39 shows HGH trimer for half-life extension using a small PEG or peptide as linker and the synthesis.

FIG. 40 shows another example of HGH trimer for half-life extension using a small PEG as linker and the synthesis.

FIG. 41 shows examples of HGH oligomer with biodegradable linker.

FIG. 42 shows an example of HGH oligomer with peptide linker prepared with recombinant technology.

FIG. 43 shows examples of HGH oligomer with terminal modifier.

FIG. 44 shows examples of HGH monomer and dimer with terminal modifier for half-life extension.

FIG. 45 shows another example of the synthesis of HGH trimer.

FIG. 46 shows an example of Exenatide monomer.

FIG. 47 shows Exenatide polymer can be degraded to release free Exenatide form

FIG. 48 shows an example Exenatide polymer having fatty acid

FIG. 49 shows an example of site specific conjugation of peptide drug to synthetic linear peptide for half-life extension

FIG. 50 shows an example of liraglutide derivative having a cleavable linker

FIG. 51 shows an example of peptide polymer drug having fatty acid

FIG. 52 shows an example of lipophilic molecules conjugated to the Exenatide via self-immolative linker

FIG. 53 shows an example of 5 Glu in Exenatide is esterized with alkyl alcohol.

FIG. 54 shows an example of shows a liraglutide conjugated with a self immolative linker and a fatty acid to bind with albumin to increase its half-life in vivo.

FIG. 55 shows exenatide conjugated with a self immolative linker and an alkyl chain to bind with albumin to increase its half-life in vivo, which release the active drug in vivo

FIG. 56 shows that the site to be adjusted for hydrolytic rate by incorporating functional group into the linker

FIG. 57 shows examples of CNP peptide conjugated to an alkyl chain with a self immolative linker

FIG. 58 shows examples of CNP peptide dimer conjugated to an alkyl chain with a self immolative linker

FIG. 59 shows examples of multimeric drug containing both CNP-22 and Extennatide.

FIG. 60 shows an example of Double filtration plasmapheresis

FIG. 61 shows another example of Double filtration plasmapheresis

FIG. 62 shows an example of ADC for SLE treatment

FIG. 63 shows example of general strucyre of Epitope(antigen)-alpha-gal conjugate

FIG. 64 shows an example of antigen-alpha-gal conjugate for SLE treatment

FIG. 65 shows examples of antigen-cell inactivating molecule conjugate

FIG. 66 shows examples of VEGF-cell inactivating molecule conjugate for cancer treatment

### DESCRIPTION OF THE INVENTIONS AND THE PREFERRED EMBODIMENT

The current invention discloses novel strategy for site specific conjugation of proteins including antibodies. Site specific antibody drug conjugation is a promising drug discovery strategy for cancer treatment; several companies (e. g. ambrx, innate-pharma and sutrobio) are working on developing new method for site specific conjugation of proteins, In one aspect, the new method in the current invention uses elevated temperature for site specific conjugation using MTgase (microbial transglutaminase, also called bacterial transglutaminase, BTG) to couple the drug/linker having amine group to the Gln of the protein. Preferred temperature is >40 degree, more preferably >45 degree but less than 75 degree. In some embodiments, the temperature is 50˜65° C. The elevated temperature can expose the previous hidden (e.g. the Gln in antibody difficult to be accessed by MTgase) functional groups for site specific conjugation.

In one example conjugation of IgG1 with Monodansylcadaverine (MDC) is catalyzed by MTgase. MDC has a primary amine and its fluorescence can be easily monitored. MDC is used here to conjugate to mAB. To purified IgG1 (1-10 mg/ml) in Tris-buffer (pH 6.5-8.5), add MDC (Sigma-Aldrich) in DMSO to final concentrations of 1-5 mM (final DMSO 2-10%). Add purified MTgase to a final concentration of 0.05-1.0 mg/ml. Incubate the reaction mixtures at 50° C. for 5 hours. Reaction is monitored by HPLC. Antigen peptide for the IgG (e.g. 5 fold excess) can be added to the reaction mix to stabilize the Fab of the antibody.

In another aspect, the new method in the current invention uses MTgase to couple the drug/linker having Gln group to the amine group of the protein (e.g. lysine or N terminal amine). The coupling can be done in either high temperature (e.g. 45˜55) or low temperature (e.g. 25-37° C.). Point mutation can be used on the protein (e.g. antibody) to introduce lysine as coupling site.

In one example, pegylation of IgG1 with 1 kDa PEG-CO-Gln-COOH or PEG-CO-Gln-Gly-NH2 is performed by MTgase catalysis. This experiment is carried out essentially the same condition as described in the example above. The MDC is replaced with MW=1 k PEG-CO-Gln-COOH (the product of HO-PEG-COOH coupling with Gln, which for an amide bond between PEG-COOH and the amine of Gln) or PEG-CO-Gln-Gly-NH2 in pH 7.0 to a final concentration of 1 to 2 mM, PEGylated IgG1 is obtained. The Gln of on the PEG couples to the amine group on the IgG1 by MTgase catalysis.

The current invention also discloses novel toxin which can be used for antibody-drug conjugate (ADC) and cancer treatment. Currently MMAE (monomethyl auristatin E) or MMAF is used for ADC as toxin to conjugate with antibody. The novel toxins in the current invention are N-substituted MMAE/MMAF. Their structures are shown below (the attachment group is where the toxin to be conjugated with):

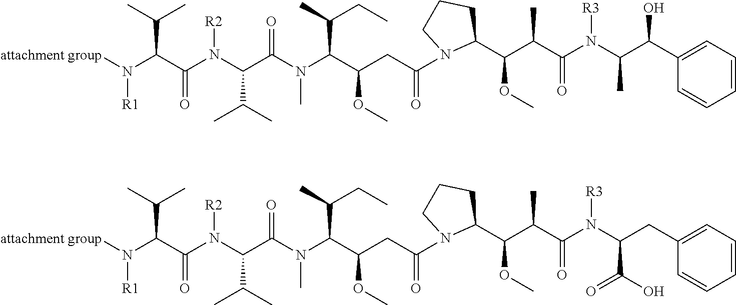

Where in R1, R2 and R3 is independently selected from the group consisting of H, C1-C8 alkyl, haloCl-C8 alkyl, C3-C8 carbocycle, aryl, X-aryl, OR21, SR21, N(R21)2, —NHCOR21 and —NHSOR2R21, X-(C3-C8 carbocycle), C3-C8 heterocycle and X-(C3-C8 heterocycle), each X is independently C1-C10alkylene.

In some examples, R1 is independently H or CH3 or CH2F or CHF2 or CF3, R2 independently is H or CH3 or CH2F or CF3 and R3 is independently H or CH3 or CH2F or CF3.

The structures also include:

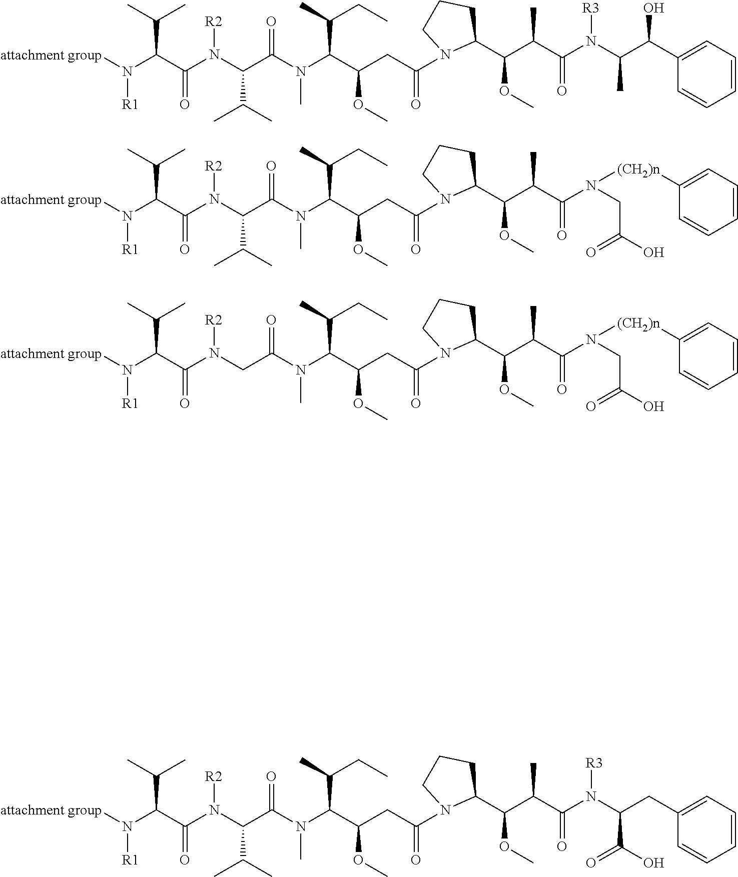

Where in R1, R2 and R3 is independently selected from the group consisting of H, C1-C8 alkyl, haloC1-C8 alkyl, C3-C8 carbocycle, aryl, X-aryl, OR21, SR21, N(R21)2, —NHCOR21 and —NHSOR2R21, X-(C3-C8 carbocycle), C3-C8 heterocycle and X-(C3-C8 heterocycle), each X is independently C1-C10alkylene, n is an integer between 1˜5.

In some examples, R1 is independently H or CH3 or CH2F or CHF2 or CF3, R2 independently is H or CH3 or CH2F or CF3 or isopropyl and R3 is independently H or CH3 or CH2F or CF3.

The attachment group is where the toxin conjugates to linker or proteins. It is the same as those used in the current MMAE/MMAF ADC.

The current invention also discloses novel strategy for antibody purification and conjugation.

Current antibody purification method uses protein A column, which is expensive and has potential risk of leaking protein A. The new strategy uses affinity column based on epitope peptide or mimotope for antibody purification by coupling epitope peptide or minotope to the solid phase support as column filler, e.g. sephadex beads. The advantages are low cost, more stable chemistry for immobilization, selectively isolating antibody with high binding affinity and removing non binding antibody/ADC, therefore increase the potency and therapeutic index of antibody or ADC. In one example: peptide NIYNCEPANPSEKNSPSTQYCYSI (SEQ ID NO: 1) is used to couple to solid phase support to make an affinity column, which can be used for Rituximab purification. The benefit of using peptide based affinity column (activated beads are commercially available) is greater than the effort of developing the peptide for each antibody. Many peptide sequence are available from literature or epitope scan for both linear and conformational discourteous epitope (e.g. from pepscan). This strategy also works for other protein drugs by using synthetic ligand (e.g. affinity peptide) for the binding site of that protein to prepare affinity column.

Furthermore, it can be used to selectively protect the reactive amino acid in the binding site of the antibody, by adding epitope peptide or mimotope (free form or immobilized) or masking peptide (e.g. those used in probody) to form the peptide-antibody complex during antibody-drug conjugation. Similarly it can be used to protect the active binding site of other type of protein by using the affinity ligand that can mask the active binding site of that protein. This method is suitable for both chemical and enzymatic conjugation, therefore provide more drug load for ADC, more conjugation reaction can be allowed (e.g. >2 types of toxin). Similar strategy is used in enzyme conjugation to keep the enzyme activity by adding enzyme substrate. Synthetic peptide is very easy to make (low cost and more stable) using synthetic peptide chemistry than making proteins. Peptide can be made in large amount easily using solid phase peptide synthesis. In one example: peptide NIYNCEPANPSEKNSPSTQYCYSI (SEQ ID NO: 1) is used to protect Rituximab during conjugating drugs to the antibody. Peptide NIYNCEPANPSEKNSPSTQYCYSI (SEQ ID NO: 1) can bind with Rituximab at its antigen binding site. By adding NIYNCEPANPSEKNSPSTQYCYSI (preferably at >2:1 ratio) to Rituximab before chemical conjugation on Rituximab, the antigen binding site of Rituximab is protected.

The current invention also discloses novel Bi specific antibody and its application. They can be used to treat cancer, pathogens, immune disorders and targeting delivery of vector (retrovirus based gene therapy).

Bi specific antibody can be in traditional monomer format: multivalent homo Fab format with a suitable length flexible linker for higher affinity (not bi specific), hetero Fab format targeting two epitope sites of the different protein on the cell/microorganism to achieve higher affinity and hetero Fab format targeting two epitope sites of the target protein to achieve higher affinity.

Bi specific antibody can also be in dimer format or trimer or higher degree oligomer format: multivalent homo Fab format with suitable length flexible linker for higher affinity (not bi specific), hetero Fab format targeting two epitope sites of the target protein for higher affinity and hetero Fab format targeting two epitope sites of the different protein on the cell/microorganism for higher affinity. Construction of this type of Bi specific antibody can be achieved using boric affinity column or lectin affinity column for mono conjugation (boric affinity column or lectin affinity column can also be used for antibody purification).

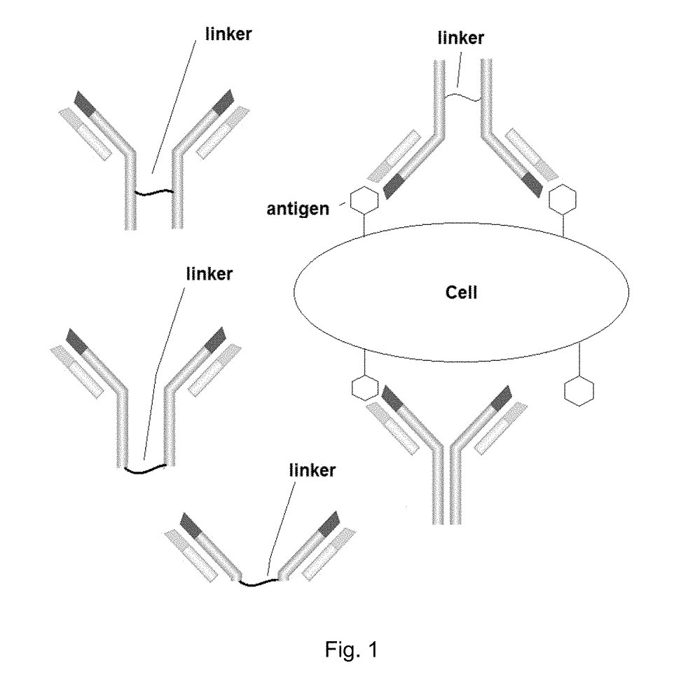

Bi Specific Antibody (BsAb) can be used for against cytoplasm target. In some embodiments, Bi specific antibody is in traditional antibody monomer format: multivalent homo Fab format with suitable length flexible linker for higher affinity. Native antibody's hinge region is not long and flexible enough therefore may not reach two antigens on the target cell. Using a flexible and suitable length of linker to connect the antibody parts will greatly increase the binding affinity (FIG. 1). The linker can be a flexible peptide linker such as poly glycine/serine or synthetic polymer such as PEG. In the current inventions the “/” mark means either “and” or “or”.

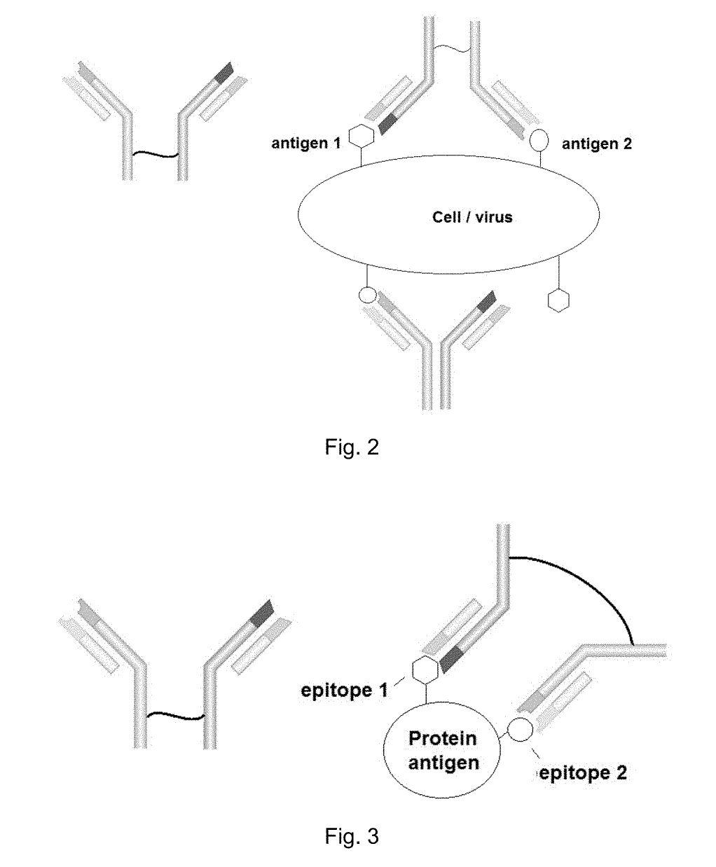

It can also be hetero Fab format targeting two antigens of the different protein on the cell/microorganism for higher affinity. Similarly, the above approach can also be applied to bispecific antibody binding to two different antigens on the cell/pathogen. The bispecific antibodies with flexible proper length linkers can be made easily to get the optimal binding of two antigens simultaneously while traditional method is time consuming (FIG. 2).

Another format is to use bi specific antibody to target the two different epitopes on the same antigen, which will also significantly increase the binding affinity (FIG. 3).

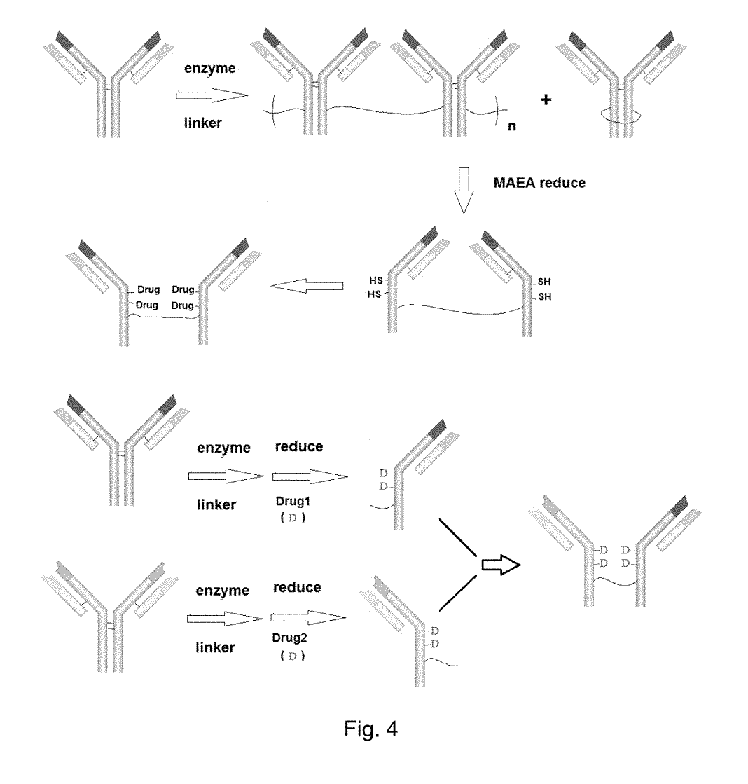

Construction of these types of Bi specific antibody: Using the selective reduction of the disulfide bond at the hinge region with 2-Mercaptoethylamine, several formats (FIG. 4) can be used to make this type of bispecific antibodies, with high yield and no concern for dimer formation to ease the industrial scale separation process. Two formats are shown below: to use some —SH reactive reagent (or mutation to remove —SH) to block the free —SH group to prevent the regeneration of —SS-bond, which will generate the traditional format bispecific antibody.

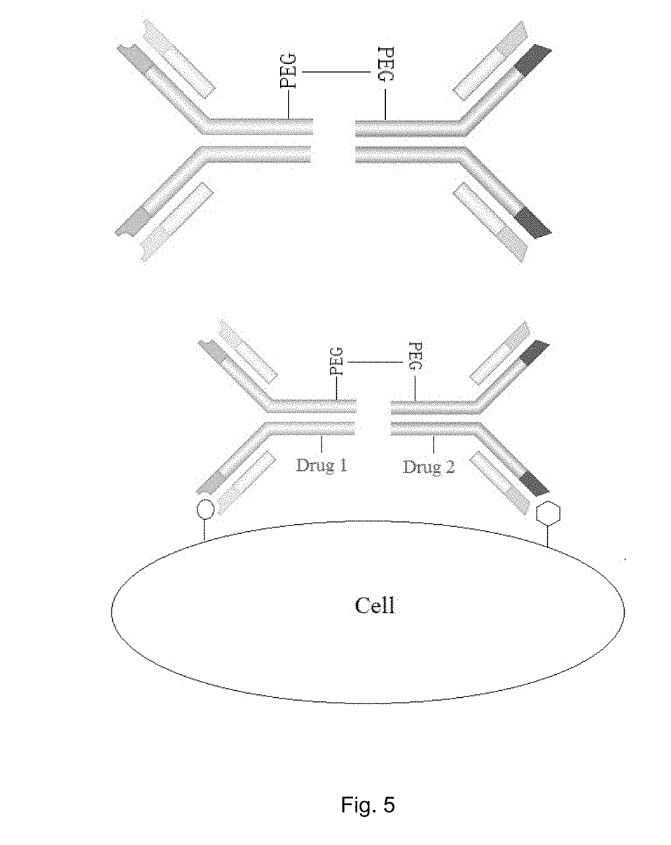

Similarly, bi specific antibody by linking two or more full size antibodies can also be used in above applications (FIG. 5) and formats and synthesized readily (FIG. 6), which may offer higher stability and higher binding affinity as shown by IgA and IgM.

Construction of this type of Bi specific antibody can be achieved using borate affinity column or lectin affinity column for mono conjugation. This strategy is also useful for antibody purification. This design uses immobilized antibody to archive high yield mono labeling of the antibody, to eliminate the potential bi-labeled antibody (generating polymerized antibody).

Immobilized protein was used to make mono PEGlated protein previously. Ion exchange resin was used to immobilize the protein. However ion exchange resin may not work for antibody to block half of FC and the binding affinity is low, which may cause exchange between two sides.

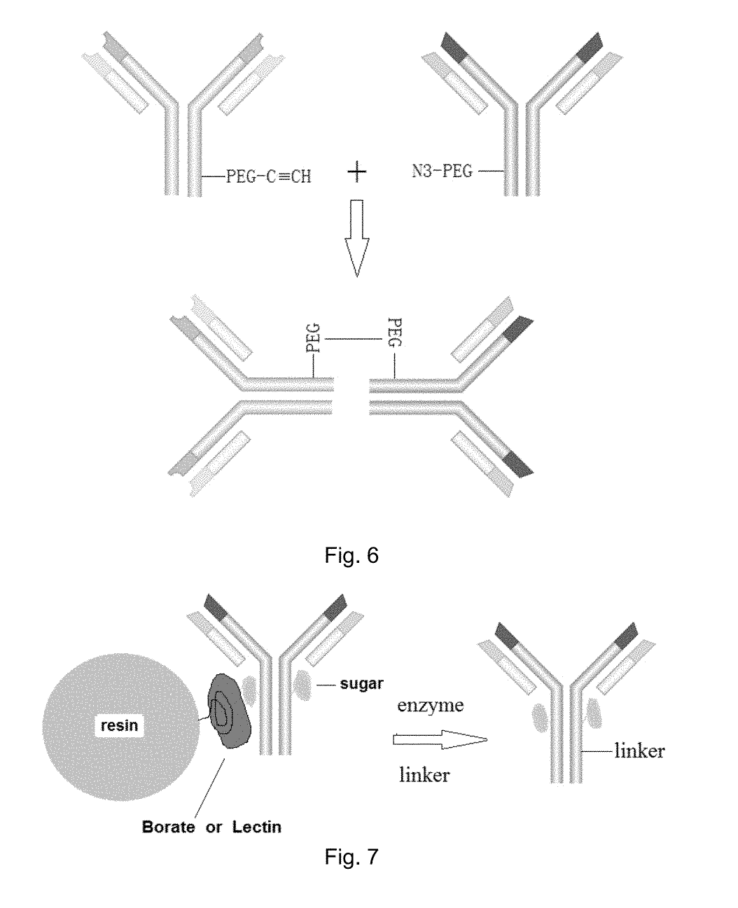

This design uses affinity group targeting the carbohydrate on the antibody to selectively protect one FC conjugation site on the antibody to achieve the mono conjugation. Suitable affinity resins include borate based affinity solid phase support or lectin based affinity phase support (FIG. 7). When one side of the antibody is protected, the other side can be selectively modified (e.g. site specific conjugation using enzyme such as mTGase).

Borate is a carbohydrate chelators and borate based column is widely used in separating carbohydrate, many are commercially available (e.g. from Sigma). Different borate also has different affinity to different sugar. Lectins are carbohydrate-binding proteins, most are from plant, which is used as antivirus/bacterial drug for animals. Different lectin has selectivity for different carbohydrate. Lectin column is also used in studying carbohydrate. Lectin or borate based resin can also be a useful tool for large scale purification of antibody drugs during ADC labeling. They can also be used for protein mono labeling other than antibody if the protein has carbohydrate modification.

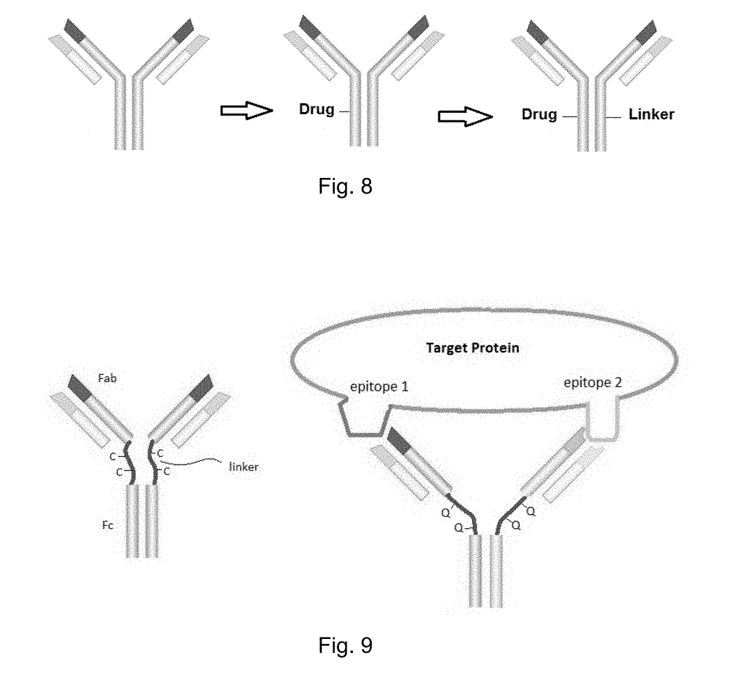

If mono labeling drug on the antibody can be done efficiently, then the later mono labeling of linker labeling can be done easily (FIG. 8).

Using ADC made of BsAb against two makers on the target cell will increase the specificity of drug delivery.

Bi Specific Antibody can be used for cytoplasm target. For example, in lupus, the key auto antibody causing the damage to the cells is the auto antibody against dsDNA. They are released from lysosome after internalization and bind with nucleus to cause cell damage. There are also many antibodies are against cytoplasm target. It is known that many cell surface receptors are reused after been internalized: suggesting it is not digested in lysosome.

Similarly, antibody against tublin can be used instead of MMAE or other toxin in the ADC. Therefore the ADC is essentially an antibody (e.g. for HER2)-antibody (e.g. for tubulin) conjugate, in another word, a bi-specific antibody. The advantage of using antibody instead of toxin as effector is that AB is much less toxic and can have high affinity and specificity, therefore less concern on side effect and toxicity due to potential release of toxin in blood circulation. Furthermore, the effector antibody may not need to target tubulin; it can be antibody against many other cytoplasm in tumor cells (e.g. tolemarase).

One issue with ADC for drug is that there are limited cell surface markers on cancers cells can be used for antibody and even HER2 is only positive in 30% patients. To expand the application of the above BS-Antibody strategy, the targets can be extended to diseases beyond cancer. There are many cytoplasm targets for many diseases and a lot of drugs are against cytoplasm targets, bi-specific antibody can be used as therapeutics against them: one AB against cytoplasm target and one against cell surface marker to help the effector AB uptaken by the cell.

The rate of internalization of antibody dimer should not be a big problem as size is not a key factor affecting internalization in many cases. A much bigger virus can be internalized easily. Even if it was a concern, monomer type Bs antibody or adding a positively charged linker can be used to improve internalization.

An antibody (against gp120)—toxin conjugate has been made to kill HIV virus infected T cell (HIV infected T cells express HIV gp 120 on T cell surface). This strategy can be applied to many other virus infections since the infected cell will express virus protein on their surface. However, toxin is toxic and has their limitations.

A more universal strategy is to use antibody-virus inhibitor conjugates instead. Many virus inhibitors are very potent and have suitable functional groups to be linked to antibody with very low toxicity to cells. For example, antibody against gp120 or CD3, CD4 can be conjugated to HIV RT inhibitor (e.g. AZT) or HIV protease inhibitor (e.g. Amprenavir) to treat HIV infection; antibody against CK18, CK19 or HBV surface antigen conjugate with RT inhibitor can be used to treat HBV infection.

A benefit of using virus inhibitor is that the antibody in ADC can target the normal cell surface marker (e.g. using ADC targeting CD3, 4 for T cell to treat HIV; using ADC targeting CK 18 for hepatic cell to treat HBV, HCV), which is prohibited for using toxin (will kill the normal cell) and the toxicity is very lower. It will also allow the inhibition of virus infecting cells before the virus protein is expressed on the host cell surface. There are applications for ADC in other diseases besides treating virus infection and cancer.

The current invention also discloses flexible antibody and bispecific antibody for site specific conjugation and better affinity. The antibody (Ab) of the current invention has a flexible linker connecting the Fab and Fc. The linker can be chemically synthesized and then conjugated to the Fab and Fc. Alternatively, the whole antibody can be expressed as a recombinant protein including the linker. The linker can be a synthetic polymer such as PEG or a flexible hydrophilic peptide (e.g. a peptide rich in Ser and Gly and Asp, 10˜50 AA).

FIG. 9 shows the flexible Ab in mono specific format (left) and bispecific format (right). The length of the flexible linker can be optimized to allow the two Fab of the resulting antibody bind to two identical epitopes at the same time or two different epitopes on the same target at the same time (for bispecific Ab). This will increase the binding affinity for the target.

It is preferred that one or more Gln (e.g. Q of antibody at right side in FIG. 9) can be incorporated into the linker, which will allow the site specific conjugation of drug to the antibody at the linker region using mTGase. Other functional group such as Cys (e.g. C of antibody at left side in FIG. 9) can be used instead of Gln for other site specific conjugation chemistry (e.g. sulfhydryl maleimide coupling).

Introducing a flexible linker having reactive amino acid into antibody provides coupling site for site specific conjugation. It also increases binding affinity, allow site specific conjugation for ADC (as shown in FIG. 10, drug D is conjugated to the antibody's Gln site specifically with mTGase), can be prepared readily with recombinant technology, can be either monospecific or bispecific antibody format.

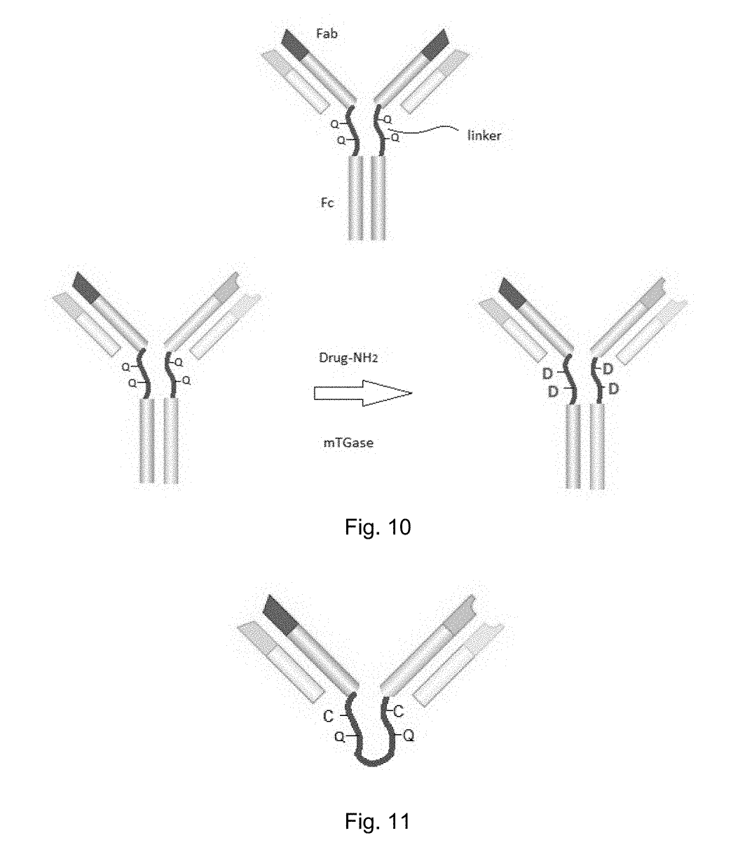

An extended flexible linker (e.g. a Ser/Gly rich peptide) provides optimal spacer to allow the two Fab to bind with two epitopes of the same target simultaneously, therefore increasing the affinity by multivalency. Reactive amino acids (e.g. Cys or Gln) can be readily expressed in the liker for site specific conjugation of ADC, the flexibility of reactive linker allow optimal conjugation efficacy, the reactive flexible linker can be readily incorporated into many other formats of bispecific antibody. Besides the format described above, this reactive flexible linker strategy can be readily incorporated into many other formats of bispecific antibody. For example, the two binding regions of the bispecific antibody without Fc can be directly linked with the reactive flexible linker (FIG. 11).

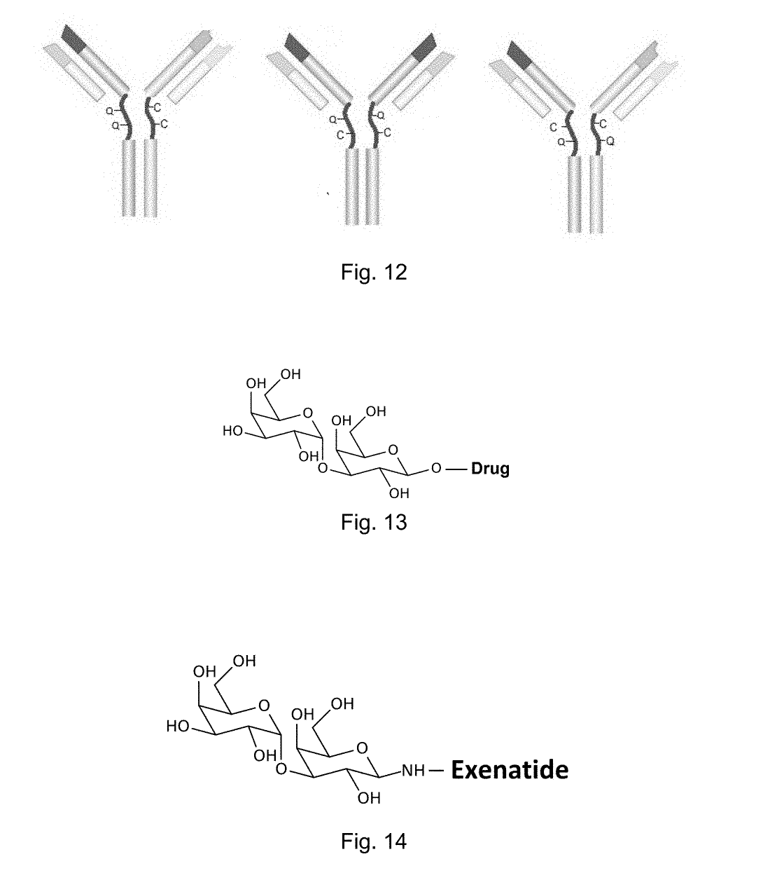

This strategy can also allow two or more types of drug to be conjugated to the antibody by introducing two or more reactive amino acids to the linker site. For example in FIG. 12, the linker contains the combination of Q and C, which allow the conjugation of different drugs using the combination of —SH based conjugation and mTGase based conjugation.

The current invention also discloses protein/peptide/small molecule drug half-life extension based on hapten-drug conjugate utilizing endogenous antibody. For example, anti-Gal antibody, binds to alpha-gal epitope (Galactose-alpha-1,3-galactose), accounts for ˜1% of total antibody in serum in all human being. The hapten-drug conjugate such as alpha-galactosyl-(optional linker)-drug conjugate will bind with endogenous anti-Gal antibody and therefore show extended half-life in vivo. An example is shown in FIG. 13.

Alpha-Gal is a small molecule, can be easily conjugated to peptide drug during peptide synthesis with minimal impact on drug structure. This method can also be applied to small molecule drug half-life extension. It may be also used for peptide vaccine to increase the half life of antigen of the vaccine. The PK (pharmacokinetics) may differ between individuals. FIG. 14 is an example design of Half-life extension using endogenous antibody and hapten-drug conjugate for GLP-1 type drug. Glucagon-like peptide-1 analogs (GLP-1 e.g. Exenatide or Liraglutide) needs daily injection for diabetes. Hapten-drug conjugate for Exenatide half-life extension can be achieved with alpha-galactosyl-(optional linker)-Exenatide. The linker can be biodegradable (e.g. self immolative linker). Besides alpha-Gal, other endogenous hapten such as L-rhamnose can also be used to conjugate with the drug to improve the half life of the drug.

Aptamer-long alkyl chain (e.g. fatty acid) conjugate can also be used for drug half life extension. Currently long alkyl chain containing compound such as fatty acid is used to conjugate with drugs (e.g. protein or peptide drug) to extend their half life by binding with albumin. However the binding is weak. An aptamer that can bind with albumin can be conjugated with one or more long alkyl chain to increase the binding affinity of this conjugate to albumin. This conjugate can be conjugated to the drug to extend its half life. Preferably the aptamer bind to albumin at the site close to the fatty acid binding site but does not block the fatty acid binding. A linker can be added between aptamer and long alkyl chain to allow optimal binding. The nucleic acid library containing alkyl chain groups can be used for SELEX to screen the aptamer containing one or more long alkyl chain that can bind with albumin. Similarly, instead of albumin binding aptamer, albumin binding peptide or other albumin binding small molecules can also be conjugated with long alkyl chain (e.g. fatty acid) with an optional linker to increase the binding of the conjugate to albumin and this conjugate can be used to attach to the drug to extend its half life.

The current also invention discloses novel strategy for antibody or aptamer construction, which can be activated by enzyme, they are called self assembly probody and protamer respectively.

Probody (e.g. those developed by Cytomx) is antibody that can be activated (having binding affinity to antigen after activation) by enzyme. Protamer is aptamer that can be activated (having binding affinity to target after activation) by enzyme.

US patent/patent applications U.S. Pat. No. 8,529,898, US 2010/0189651(U.S. Ser. No. 12/686,344), US20130315906 (U.S. Ser. No. 13/872,052) and US20140010810 (U.S. Ser. No. 13/923,935) disclosed antibody construction called probody that can be activated by enzyme.

The probody in the prior art are activatable binding polypeptides (ABPs, e.g. antibody), which contain a target binding moiety (TBM), a masking moiety (MM), and a cleavable moiety (CM) are provided. Activatable antibody compositions, which contain a TBM containing an antigen binding domain (ABD), a MM and a CM are provided. Furthermore, ABPs which contain a first TBM, a second TBM and a CM are provided. The ABPs exhibit an “activatable” conformation such that at least one of the TBMs is less accessible to target when uncleaved than after cleavage of the CM in the presence of a cleaving agent (e.g. enzyme) capable of cleaving the CM. Further provided in the prior art are libraries of candidate ABPs, methods of screening to identify such ABPs, and methods of use. Further provided are ABPs having TBMs that bind VEGF, CTLA-4, or VCAM, ABPs having a first TBM that binds VEGF and a second TBM that binds FGF, as well as compositions and methods of use. The prior art disclosure provides modified antibodies which contain an antibody or antibody fragment (AB) modified with a masking moiety (MM). Such modified antibodies can be further coupled to a cleavable moiety (CM), resulting in activatable antibodies (AAs), wherein the CM is capable of being cleaved, reduced, photolysed, or otherwise modified. AAs can exhibit an activatable conformation such that the AB is more accessible to a target after, for example, removal of the MM by cleavage, reduction, or photolysis of the CM in the presence of an agent capable of cleaving, reducing, or photolysing the CM.

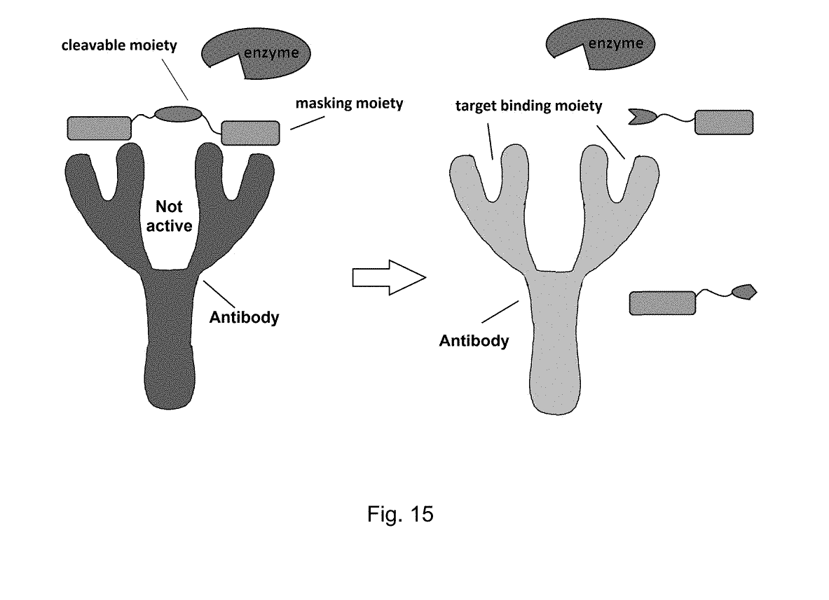

The current invention discloses novel probody format. In the prior art, the masking moiety MM is covalently conjugated to the target binding moiety TBM (e.g. antibody, receptor, ligand for receptor such as VEGF). In the current invention, the difference is that the masking moiety MM is not covalently linked to the TBM (e.g. antibody, receptor, ligand for receptor such as VEGF). The cleavable moiety (CM) connect two MM instead of connecting the MM with the TBM in the prior art. Optionally a linker/spacer (e.g. a peptide or PEG) can be added between the MM and CM to allow optimal binding of two MM to the two Fab sites (or other binding moieties such as VEGF). The TMB such as antibody, MM and CM sequence can be essentially the same as these in the prior art disclosure except the linking between them is different as described above. The tandem MM strategy in the prior art can also be applied (FIG. 15). The probody in the current invention is a bound complex instead of a single molecule as that in the prior art. This strategy allows the use of the current available antibody or protein without the need to develop a new conjugate, therefore simplify the drug development process. The enzyme will cleave the CM and activate the TBM by exposing the previously blocked binding sites. One can either use the pre-formed complex or give the patient the two components separately to allow the complex form in vivo.

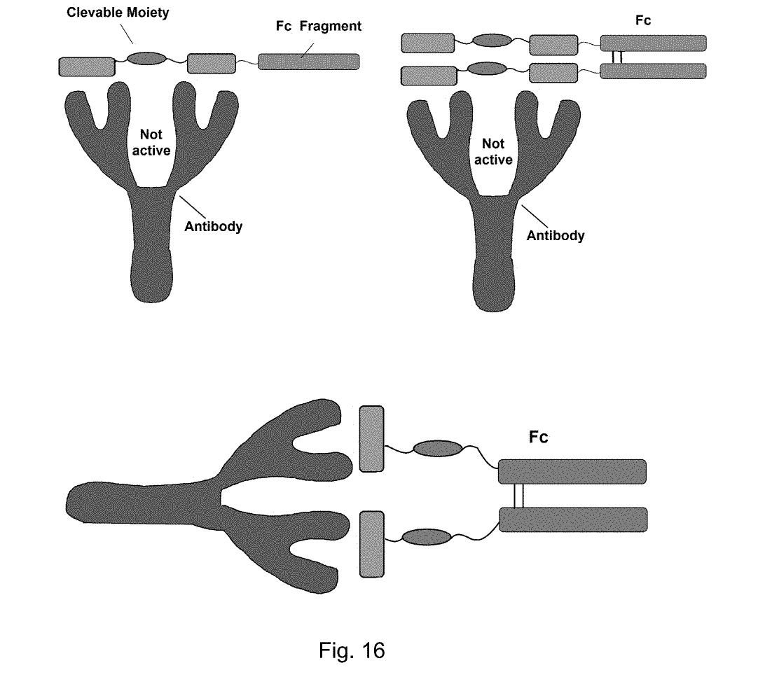

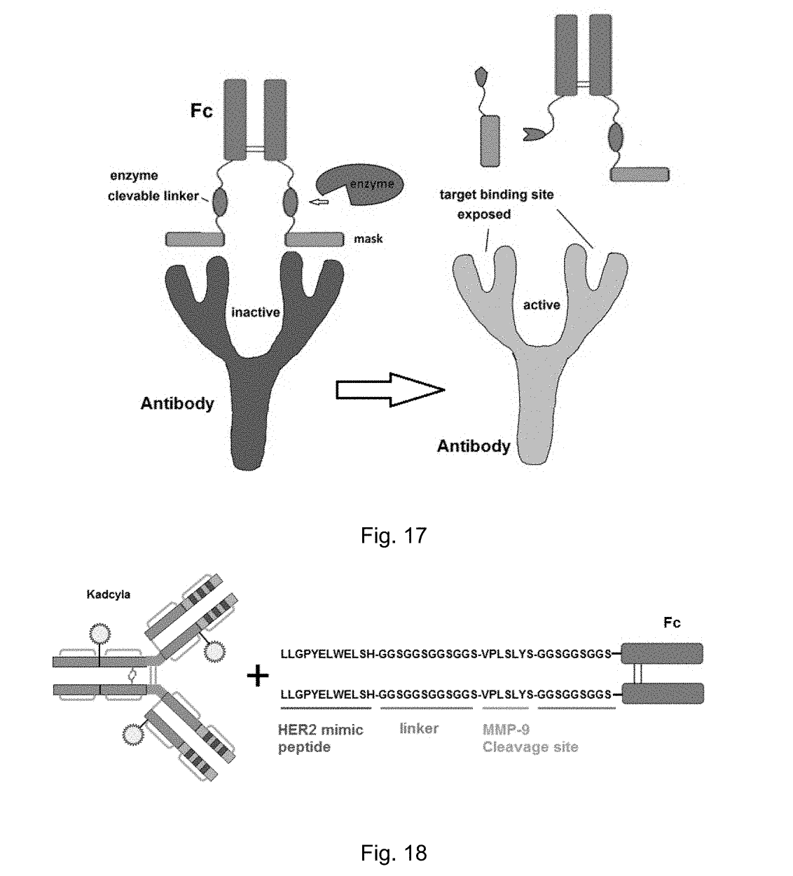

Preferably antibody Fc or its fragment (e.g. single chain) can be connected to the MM (either by chemical conjugation or fusion/expression) to increase its half life (examples see FIGS. 16-17). Besides Fc tag, other half life extender (e.g. PEG, albumin, lipophilic tag, Xten, carboxyl-terminal peptide (CTP) of human chorionic gonadotropin (hCG)-beta-subunit) currently used to extend in vivo protein half life can also be attached to the MM covalently to reduce its in vivo inactivation/elimination (FIG. 16-17). In some embodiments the antibody can be engineered that the binding of ligand (masking moiety) with antibody does not activate complement. The antibody can have mutations that preclude binding to FcγR and/or C1q. The antibody (or other TBM) can be conjugated with drugs as a targeted drug delivery system. Excess amount of cleavable moiety (CM)—MM conjugate can be used to inhibit the antibody (or other TBM) binding completely.

In one example (FIG. 18), Trastuzumab emtansine self-assembly probody is disclosed. LLGPYELWELSHGGSGGSGGSGGSVPLSLYSGGSGGSGGS (SEQ ID NO: 2) containing a HER2 mimic peptide, linker peptides and MMP-9 substrate peptide is fused with Fc, which forms a self assembly complex with Trastuzumab emtansine to block its binding affinity with HER-2 when no MMP-9 is present. The matrix metallopeptidase 9 (MMP-9) cleave the Fc-Mask peptide; release the active Trastuzumab emtansine (Kadcyla) to bind with HER2 on the tumor cell for targeted cancer therapy.

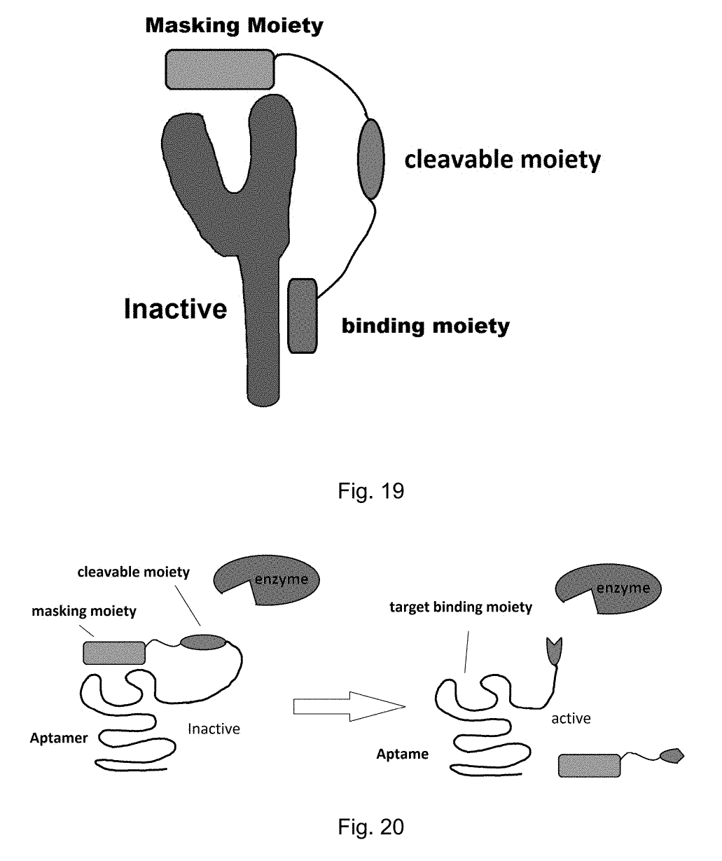

The two MM can also be heterogenic. One binds with the active site of the protein (e.g. the Fab or binding part of the protein), another bind with another part of the protein (non-TBM binding/active site). In this scenario, sometimes one of the MM is not a masking moiety anymore; it is essentially a binding moiety (FIG. 19). In FIG. 19, the masking moiety is a binding ligand for TBM while the binding moiety is protein A that binds with the Fc of the antibody.

The current invention also discloses novel protamer that can be activated by enzyme to restore its binding affinity. It is similar to probody except the activatable binding polypeptides (e.g. antibody) is replaced by an aptamer. The designs of protamer are illustrated in the FIG. 20. In one format, the aptamer is conjugated with a CM and then a MM covalently. The sequence of the CM can be the same as those used in probody. The MM is an affinity ligand (e.g a peptide that can bind with the aptamer binding domain or a complementary nucleic acid sequence) to the aptamer that can block the binding affinity of the aptamer. When the activating enzyme (or other condition such as low pH or recuing environment or light) is not present, the target binding affinity of the protamer is blocked by the masking moiety. When the enzyme is present, the enzyme will cleave the CM and activate the aptamer by exposing the previously blocked aptamer binding site.

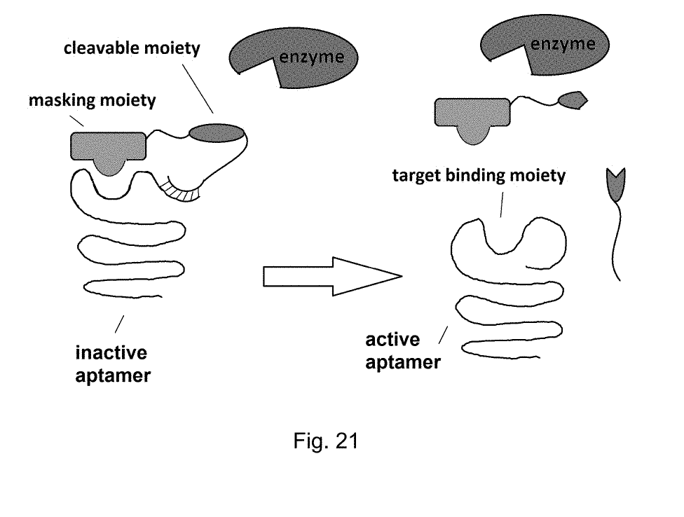

Alternatively, the CM can also be linked to the aptamer non-covalently, similar to the novel probody described in the current invention. For example (FIG. 21), the CM is linked to a nucleic acid sequence that can bind with the aptamer, therefore bind with the aptamer non- covalently.

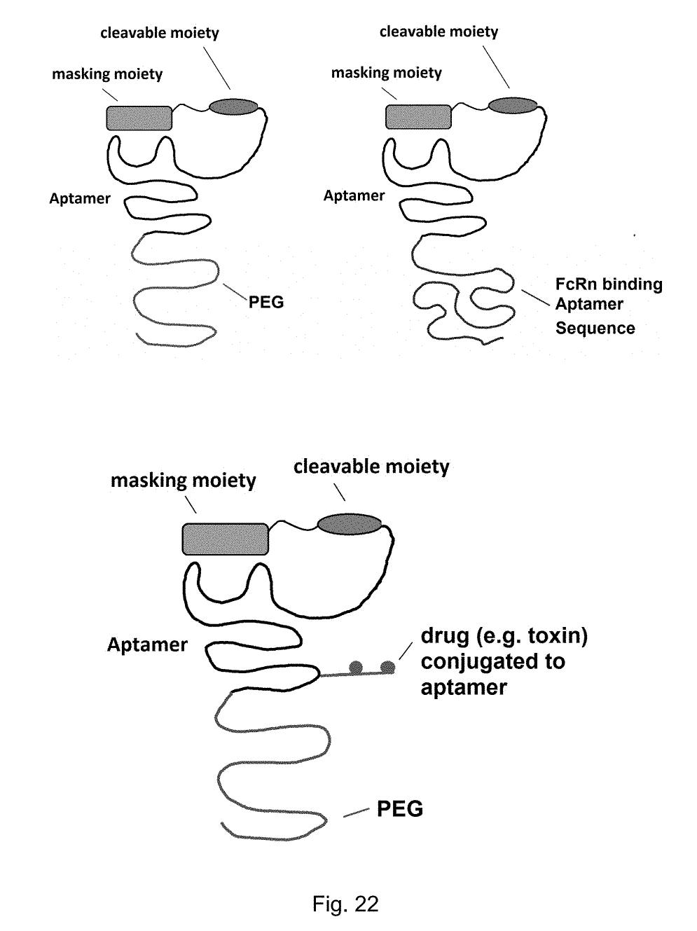

The aptamer can also be conjugated with a drug (e.g. toxin, radioactive element, chelater-radioactive element complex) to act as a targeted drug delivery system similar to the antibody drug conjugate. The aptamer can also be conjugated with a PEG or Fc domain or other polymer (e.g. Xten from Amunix) or tag (e.g. an affinity tag that can bind with albumin) to extend its in vivo half life. The aptamer can also have a binding sequence (made of another nucleic acid sequence) mimic the Fc domain of antibody to allow the recycle of the aptamer. This sequence is essentially an aptamer that mimic the function of Fc domain that can bind with FcRn at acidic pH of (<6.5) but not at neutral or higher pH. Examples are shown in the FIG. 22.

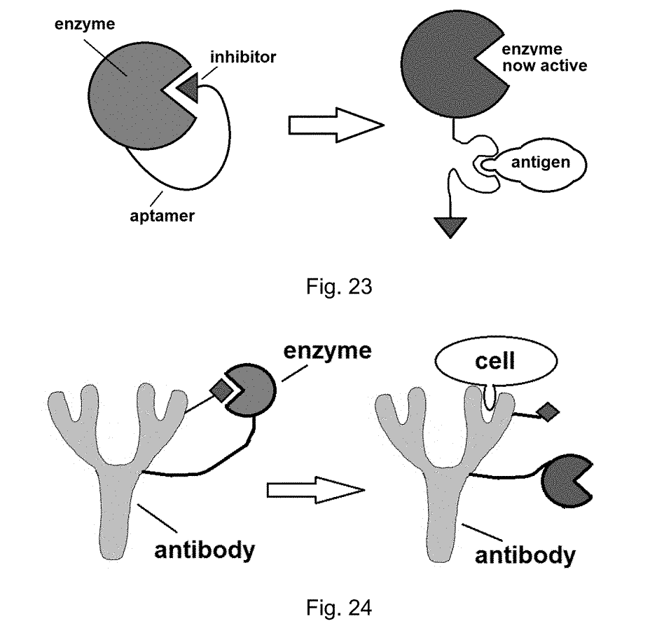

The current invention discloses novel strategy for enzyme construction which is called Binding Based Prozyme. Binding Based Prozyme is enzyme conjugated with affinity ligand (e.g. aptamer or antibody). When its affinity ligand does not bind with the target, the enzyme has low or no activity. When it binds with the target, the enzyme is activated to show high catalytic activity (FIG. 23). The affinity ligand is covalently coupled to the enzyme; the affinity ligand is also coupled with an enzyme inhibitor (e.g. a molecule that can mask the enzyme catalytic center) or a molecule that can block the enzyme's active site. When the target molecule (antigen) is not present, the enzyme inhibitor binds with the enzyme to block the enzyme's activity. When the target molecule (antigen) is present, the aptamer bind with the antigen and the conformation change of the aptamer due to binding inhibits the binding of the enzyme inhibitor with the enzyme, therefore exposes the active enzyme catalytic site and restores the enzyme activity.

In one example, glutathione S-transferase-PEG 20-CGA GAG GTT GGT GTG GTT GG (SEQ ID NO: 3)-fluorescein-3′ is made by coupling 5′-PEG 20-CGA GAG GTT GGT GTG GTT GG (SEQ ID NO: 3)-fluorescein-3′ having a —COOH group at the PEG end with the amine group on the enzyme using EDC. -CGA GAG GTT GGT GTG GTT GG (SEQ ID NO: 3)—is a thrombin-binding DNA aptamer. Fluorescein is an inhibitor of glutathione S-transferase. The resulting conjugate has low enzyme activity when there is no thrombin and has high enzyme activity when thrombin is present.

FIG. 24 shows the resulting steric hindrance from binding of antibody with the antigen releases the active enzyme from its inhibitor therefore restores the enzyme activity. The enzyme inhibitor is conjugated close to the antibody's antigen binding site and the enzyme is conjugated to the antibody with a linker. When the antigen is not present, the enzyme is blocked by the inhibitor. When the antigen is present, the antibody will bind with the antigen and the resulting steric hindrance from binding of antibody with the antigen prevents the binding of the inhibitor with the enzyme, therefore restore the activity of the enzyme.

Another example is a sialidase-antibody conjugate. The antibody is a therapeutical antibody against cancer such as herceptin. The sialidase is engineered to have an antibody binding epitope peptide region or its mimic (e.g. HER2 epitope mimic) expressed close to its catalytic center. The sialidase is linked with the antibody (e.g. at C terminal of its Fc) with a flexible linker having suitable length that allows the antibody bind with the epitope mimic region of the sialidase in an intra molecule format therefore block the enzyme activity of sialidase. When the antibody reach the cancer cell, the epitope on the cancer cell will replace the epitope mimic at the sialidase for antibody binding therefore expose the catalytic center of the sialidase, restore its enzyme activity. The activated sialidase can enhance the anticancer efficacy of the antibody. In some embodiments the epitope at the sialidase is not close to its catalytic center and the binding with antibody induce conformational change which inactivate the enzyme, once the binding is removed by the competing binding of the cancer cell epitope, the enzyme become active again.

This strategy can be used to provide therapeutic enzyme conjugate that become activated enzyme when it binds with certain target, therefore provides better target specificity. For example, the affinity ligand can bind with certain cell or pathogen surface marker and the enzyme can produce certain biological effect to the cell or pathogen. When there is no target cell/pathogen present, the enzyme is inactive, when the maker bearing cell/pathogen is present, the enzyme conjugate bind with the cell/pathogen and the enzyme become active, produce therapeutical effect to the cell or pathogen. In one example, the affinity ligand is an aptamer or antibody against HER2, the enzyme is a protease or an enzyme that can convert an anti caner prodrug to its active form. This Prozyme can be used to selectively inactivate the HER2 positive cancer cells. In another example, the affinity ligand is an aptamer or antibody against gp-120, the enzyme is a hydrolase that can damage the virus particle. This Prozyme can be used to selectively inactivate HIV virus.

Alternatively, the affinity ligand can bind with one part of the target macromolecule (or its complex) and the active enzyme can act on the other part of the macromolecule (or its complex), when the target macromolecule (or its complex) is present, the enzyme will be active and act on the target macromolecule (or its complex). In one example, the target is amyloid plaques. The affinity ligand can bind with amyloid plaque and the enzyme is a hydrolase that can cleave peptide bonds. This Prozyme can be used to hydrolyze amyloid plaques. This method also provides a new method to develop new enzyme, by coupling a specific ligand to enzyme that has a broad substrate spectrum. The resulting enzyme will have higher selectivity: only act on the target that can bind with the affinity ligand.

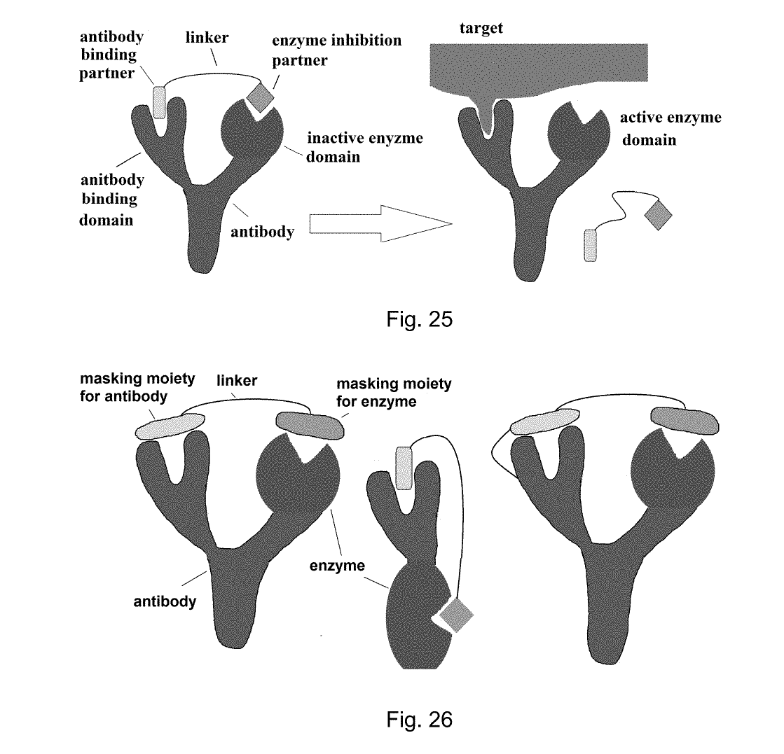

Another format (FIG. 25) is to use an ABP (antibody binding partner)-linker-EIP (enzyme inhibition partner) to form a non-covalent complex with the antibody-enzyme fusion protein, in which the enzyme domain is inactivated by the EIP. The ABP can be the antigen or MM used in the probody. The EIP can be an enzyme inhibitor or a masking molecule that mask the enzyme active center. The linker length is optimized to ensure the maximal binding of ABP and EIP to the fusion protein. When the antibody binding target is present, the ABP-linker-EIP is displaced and the enzyme activity is restored. ABP-linker-EIP can be added in excess amount to inhibit the enzyme activity to the desired level when binding target is not present. In some embodiments, the ABP can also be conjugated to the antibody, which will result in a covalent complex with the antibody-enzyme fusion protein. Examples of possible formats are shown in the FIG. 26. Besides antibody or antibody fragment, other affinity ligand for the target such as aptamer can also be used to conjugate/fuse with the enzyme.

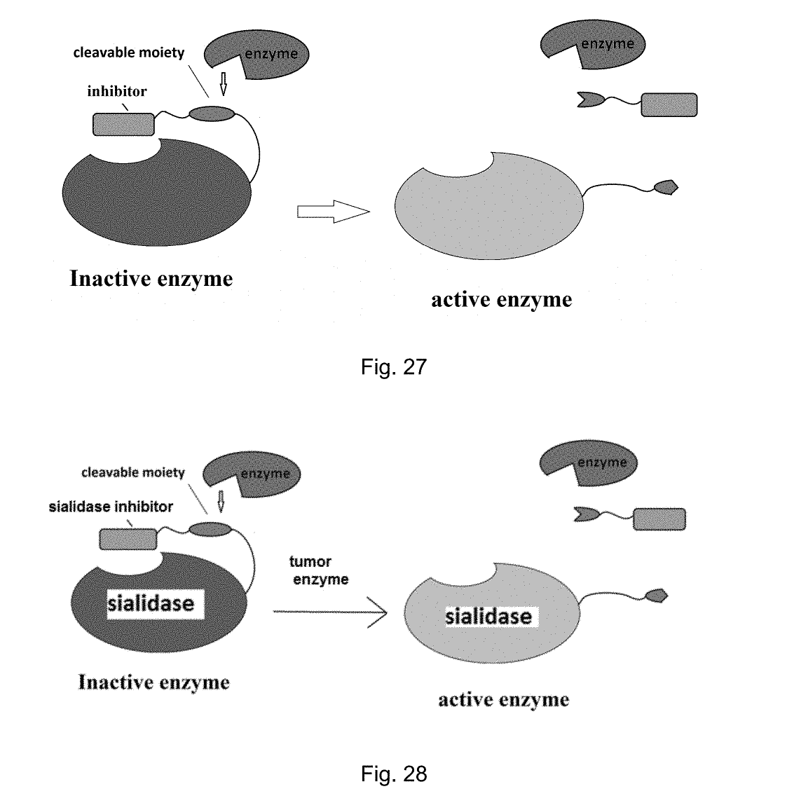

The current invention discloses novel strategy for enzyme construction which is called Cleavage Based Prozyme. Cleavage Based Prozyme is an activatable enzyme, which contains an active enzyme moiety (or a catalytic domain of an enzyme) conjugated with enzyme inhibitor moiety via a second enzyme (or other condition such as low pH or reducing environment) cleavable moiety (CM), a mechanism similar to probody. When there is no second enzyme or suitable cleavage condition, the active enzyme moiety binds with the enzyme inhibitor moiety or is blocked by the enzyme inhibitor moiety, therefore has low or no activity. When there is second enzyme or other cleavage condition, the enzyme cleavable moiety is cleaved to release the enzyme inhibitor from the enzyme, therefore the enzyme is activated to show high catalytic activity (FIG. 27). The second enzyme can be either the same as the activatable enzyme or an enzyme with different catalytic activity.

The cleavable moiety is covalently coupled to the enzyme; the cleavable moiety is also coupled with an enzyme inhibitor (e.g. a molecule that can mask the enzyme catalytic center). In one example, glutathione S-transferase-PEG 20-CCCCAAA-fluorescein-3′ is made by coupling 5′-PEG 20-CCCCAAA-fluorescein-3′ having a —COOH group at the PEG end with the amine group on the enzyme using EDC. -CCCCAAA is DNA fragment which can be cleaved by DNase. Fluorescein is an inhibitor of glutathione S-transferase. The resulting conjugate has low enzyme activity when there is no DNase and has high enzyme activity when DNase is present.

This strategy can be used to provide therapeutic enzyme conjugate that become activated enzyme when it is close to a target having the second enzyme, therefore provides better target specificity. For example, the second enzyme can be on the surface of or inside certain cell or pathogen and the enzyme can produce certain biological effect to the cell or pathogen. When there is no target cell/pathogen present, the enzyme is inactive, when the second enzyme bearing cell/pathogen is present, the enzyme conjugate will be cleaved by the cell/pathogen and the enzyme become active, produce therapeutical effect to the cell or pathogen. In one example, the cleavable moiety is a special peptide sequence that can be cleaved by a protease, the enzyme is an esterase that can convert an anti caner prodrug to its active form. This prozyme can be used to selectively inactivate the said protease rich cancer cells.

Furthermore, the prozyme can be conjugated to or fused to an affinity ligand (e.g. an antibody) to provide further selectivity. In one example, the antibody is an antibody against HER2, therefore the Prozyme-antibody conjugate can be used to kill HER-2 positive cancer cells. In one example, the cleavable moiety and the linker connecting antibody with the enzyme (e.g. those currently used in ADC drugs) are substrate of the enzyme in lysosome. After endocytosis, the prozyme-antibody conjugate in the lysosome is cleaved to release the active enzyme to kill the cancer cell. Hydrophilic carbon chain can be introduced into the conjugate to help breaking the lysosome membrane.

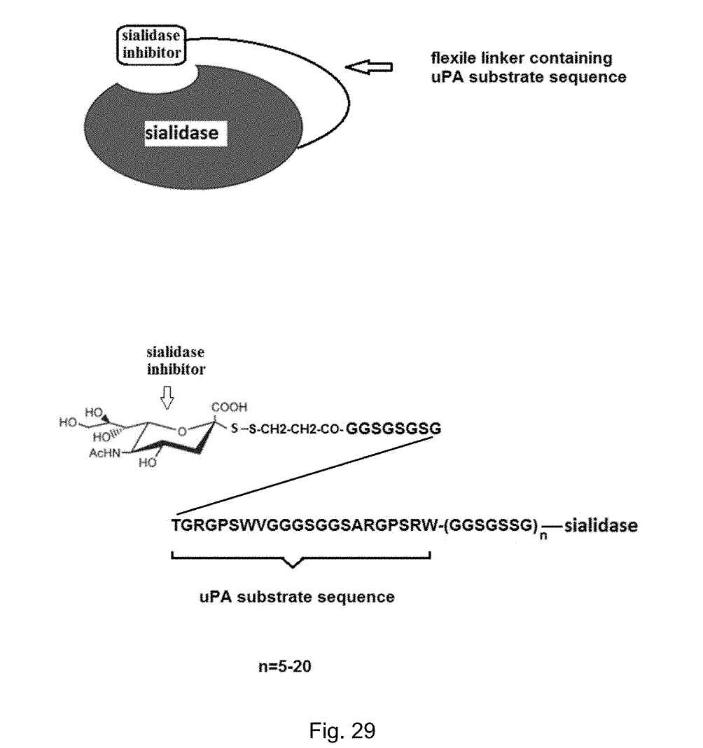

In some embodiments, the enzyme activatable prozyme strategy is applied to sialidase (neuraminidase). Tumor cell surface has high density of sialic acid, which protects the tumor cell from attack of the immune system and antibody drugs. Removing the cancer cell surface sialic acid can improve the efficacy of immune therapy and immune cell cytotoxicity against tumor cell. Antibody-sialidase conjugate can remove tumor cell surface sialic acid, improves the complement activation and ADCC of antibody drug (e.g. Herceptin) by activating NK cell. The prozyme strategy can be applied to sialidase for cancer therapy. The prozyme can be administered to the patient (e.g. 10 mg˜500 mg intravenous injection daily or weekly) to increase their immune response against cancer cells from immune cell as well as antibody drugs. As shown in FIG. 28, the sialidase is covalent linked with a flexible linker, the linker contains one or more tumor enzyme cleavable peptide sequence or non-peptide substrate (e.g. an oligosaccharide), the linker is further linked with a sialidase inhibitor. The whole structure can be either expressed as a recombinant protein or chemically conjugated together. Examples of the tumor enzyme can be found in the probody from Cytomx Inc., such as legumain, plasmin, TMPRSS-3/4, MMP-9, MT1-MMP, cathepsin, caspase, human neutrophil elastase, beta-secretase, uPA, or PSA. The cleavable moiety used in the probody from Cytomx Inc. can be used as the cleavable peptide sequence. The flexile linker contains flexible peptide sequence or other flexible polymer (e.g. PEG) at optimal length to allow the sialidase inhibitor bind with the sialidase when the linker is not cleaved. The sialidase can be either human sialidase or bacterial sialidase or virus sialidase such as flu sialidase, V. Cholerae sialidase, NEU1, NEU2, NEU3 and NEU4. FIG. 29 shows an example of sialidase prozyme to treat cancer. It can be activated by uPA in the tumor therefore selectively cleave the sialic acid on the tumor cells. It contains a sulfur substituted sialic acid as sialidase inhibitor, which connect to a flexible linker with disulfide bond. The flexible linker contains an uPA cleavable sequence. Another end of the linker is connected to the N terminal of sialidase either by chemical conjugation or expression. An example of the flexible linker for uPA is:

(SEQ ID NO: 6)
-GGSGSGSG-TGRGPSWVGGGSGGSARGPSRW-GGSGSSG-

The GS rich peptide region before and/or after the uPA substrate region in the above sequence can be repeated (e.g. 5˜20 times) to give the optimal linker length to allow the intra molecular binding of the inhibitor with the sialidase.

The sialidase can also be conjugated with one or more affinity ligand to the therapeutical antibody (e.g. an antibody against cancer cell such as Herceptin). It will bind to the therapeutical antibody and cleave the sialic acid on the cancer cells once the anti cancer antibody bind with cancer cells. This will provide targeted delivery of sialidase and increase therapeutical efficacy of the therapeutical antibody. It can be either pre-mixed with antibody to form the binding complex or injected to the patient separately to allow the sialidase-therapeutical antibody complex form in vivo. The affinity ligand can bind with either with Fc or Fab or Fab′ of the therapeutical antibody but should not block the binding of the therapeutical antibody to its target (non-neutralizing). Preferably the ligand binding with the therapeutical antibody should not inhibit the ADCC of antibody and should not inhibit complement activation. The antibody binding ligand can either be peptide, antibody, antibody fragment, aptamer or small molecules. For example, when anti cancer therapeutical antibody is IgG containing humanized Fab, a non-neutralizing antibody or its Fab′ or Fab fragment against human IgG Fab region can be used to conjugate with sialic acid. In some embodiments, the antibody against human IgG Fab used for sialidase conjugation can be those used as secondary antibody against Fab in ELISA, for example, it can be Human IgG Fab Secondary Antibody (mouse anti human SA1-19255) from ThermoFisher or Mouse Anti-Human IgG Fab fragment antibody [4A11] (ab771) from Abcam or their F(ab)/Fab′/F(ab′)₂ fragments. The anti-Human IgG Fab antibody or its fragment can be conjugated to the sialic acid via a linker (e.g. PEG or flexible peptide) either chemically or by expression. The sialidase can be either active sialidase or the prozyme form sialidase.

When the therapeutical antibody is an antibody against pathogens such as bacterial, the sialidase conjugate in the current invention can also be used to increase the efficacy of treating pathogens by removing the sialic acid on the pathogen surface.

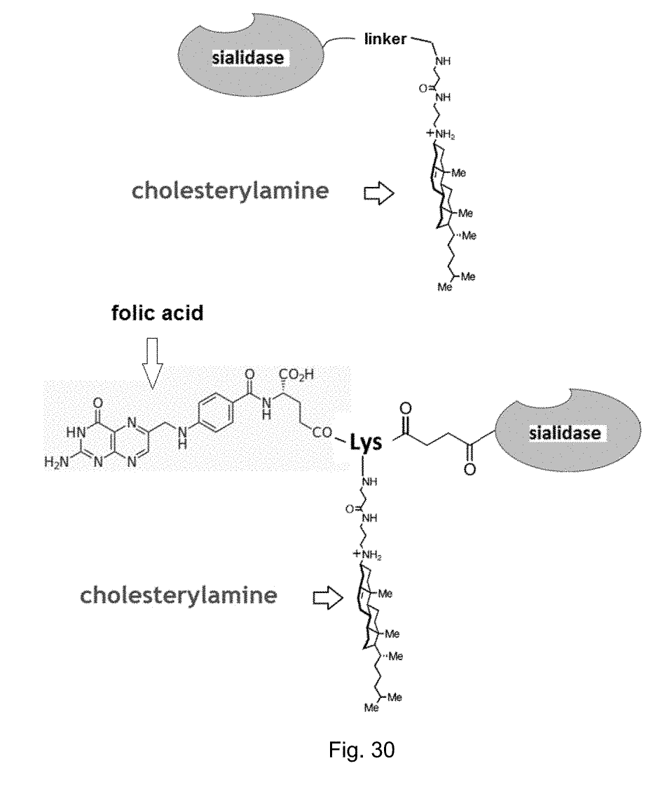

The sialidase (either as active enzyme or in prozyme form) can also be conjugated with one or more lipid type molecule such as Sphingolipids or Cholesterol derivative (e.g. 3β-cholesterylamine). This will help anchor the sialidase on the cell surface and extend its half life by endosome recycling. This kind of lipid-sialidase conjugate can be injected to the tumor directly to treat cancer. The sialidase can also be conjugated with one or more peptide or small molecule affinity ligand to the cancer cell to increase its targeting. Example of suitable affinity ligand include folic acid derivatives and RGD peptide/peptidomimetic. The sialidase-affinity ligand conjugate can further include one or more lipid moiety as described above. FIG. 30 shows example of sialidase-lipid conjugate and sialidase-lipid-folic acid conjugate for cancer treatment.

The current invention also discloses biological active protein that can be used as potential drug in oligomer format (e.g. trimer format, which connects 3 proteins with either cleavable or non-cleavable linkers) and its application in HGH oligomer (e.g. trimer) to increase their in vivo half life and potency.

Modification of proteins with hydrophilic polymers is an effective strategy for regulation of protein pharmacokinetics. However, conjugates of slowly or non-biodegradable materials, such as poly ethylene glycol (PEG), are known to cause long-lasting cell vacuolization when its MW is high, in particular in renal epithelium. Conjugates of more degradable polymers, e.g., polysaccharides, have a significant risk of immunotoxicity. Polymers that combine complete degradability, long circulation in vivo, and low immuno and chemical toxicity would be most beneficial as protein conjugate components. In one aspect the current invention uses biodegradable linker to connect PEG block polymer (or other synthetic polymer) to generate large MW biodegradable PEG (or other synthetic polymer). The resulting big MW PEG (or other synthetic polymer) can break into small PEG (or other synthetic polymer) to increase drug potency/PEG (or other synthetic polymer) clearance and reduce toxicity of large PEG (or other synthetic polymer). Proteins with MW<70 K can be rapidly cleared by kidney. People use PEG to conjugate to proteins to increase its MW to reduce the kidney clearance rate. However large

PEG (MW>40K) can cause kidney damage and has high viscosity which makes protein drug injection difficult. Examples of biodegradable linker include peptide, ester, polylactic acid, carbohydrate, polyal (e.g. those in U.S. Pat. No. 8,524,214), biodegradable hydrophilic polyacetal, poly (1-hydroxymethylethylene hydroxymethylformal, polyphosphate, Mersana's Fleximer® polymer and etc. Peptide that can be cleaved with endogenous peptidase/protease and those cleavable linkers used in ADC (e.g. hydrazone linker, disulfide linker, peptide linker such as-Val-Cit-) can also be used to connect small PEG fragment/blocks (or other synthetic polymer), which can undergo enzyme cleavage, acidic (e.g. proton-catalyzed hydrolysis at lysosomal pH), proteolytic or redox cleavage.

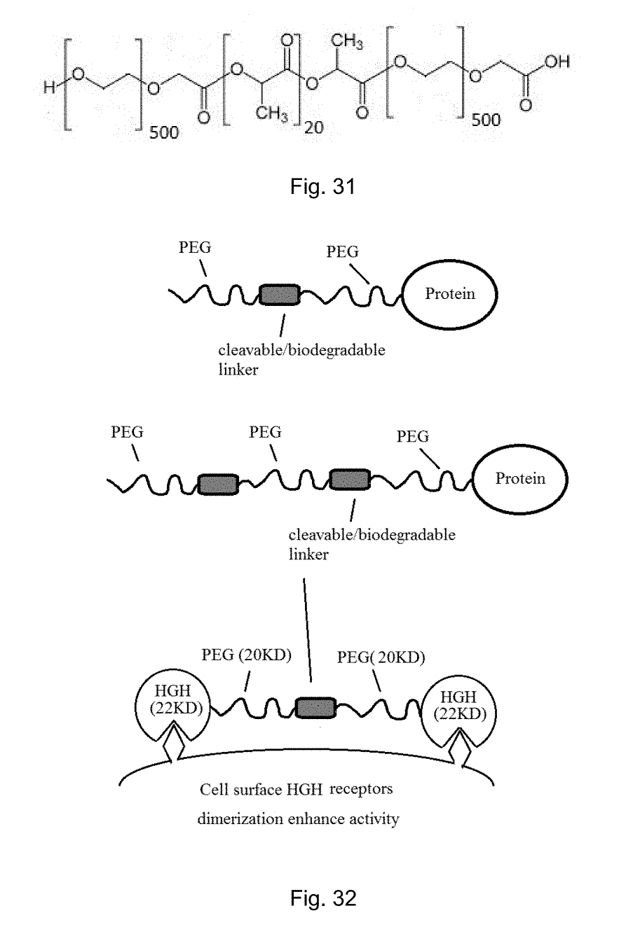

When PEG is used It has the following general structure: (PEG-biodegradable linker)_{N}-protein (N is an integer). Optionally there is a attachment moiety (e.g. a chemical bond or conjugation linker) between the (PEG-biodegradable linker)_{N} and the protein to connect them together. One example is given in the FIG. 31, which is a block polymer made of two PEG blocks connected with a biodegradable polylactic acid. One end of the PEG has a —COOH group, which can be used to couple to the amine group of the lysine on the protein surface. Other synthetic polymer such as poly vinyl alcohol can also be instead of PEG.

In another example HGH dimer is constructed. Human growth hormone (HGH, MW=22K) needs daily injection due to its fast kidney clearance. Biodegradable HGH dimer can be used as a better alternative: HGH-PEG(20K)-cleavable linker-PEG(20K)-HGH MW=85K>70K (MW cutoff for kidney clearance). In one embodiment the PEG has an amine terminal, which can couple to the Gln on the HGH by mTgase. The FIG. 32 illustrates different formats of biodegradable PEG and the biodegradable HGH dimer.

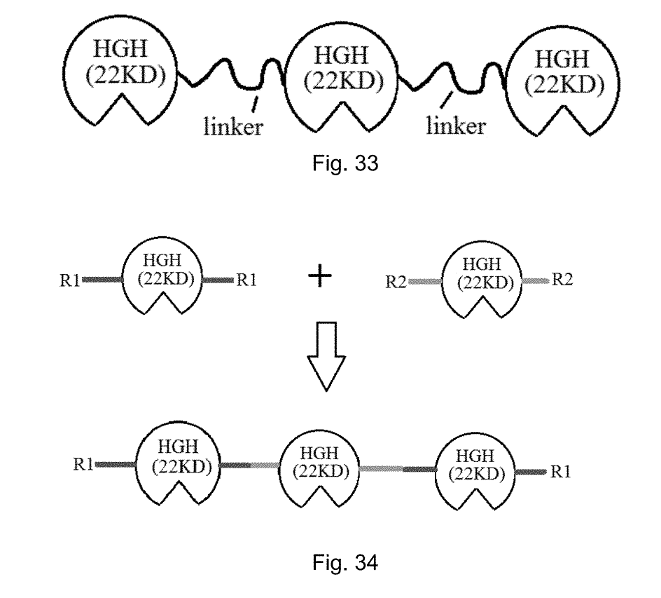

Alternatively, 3 proteins can be covalently connected to form a trimer with two linkers, which will further increase its size and molecular weight therefore extend its half life in vivo. The linker can be either biodegradable or non biodegradable. Preferably the molecular of the resulting trimer is greater than 60 KD. In some embodiments it is greater than 70 KD. The preferred linker should have a preferred molecular weight that make the total trimer >60 KD. The linker can be PEG, peptide or other biologically acceptable linker. FIG. 33 shows an example of HGH trimer which can extend HGH in vivo half life.

The two linkers connecting the 3 HGH can be the same. For example, it can be a PEG or a hydrophilic peptide (e.g. peptide rich of Ser, Thr, Glu, Asp) having a MW between 500˜15 KD.

FIG. 34 shows another example of the HGH trimer and its preparation. Each HGH has two modifications resulting in two reactive groups. R1-PEG-NH2 and R2-PEG-NH2 can be site specifically conjugated to HGH separately by MTgase. R1 and R2 are reactive groups (e.g. those in click chemistry, —SH/maleimide pair and etc) that can conjugate together specifically to form a covalent bond. Next the resulting two HGH are mixed and the covalent bond is formed connecting R1 and R2. To ensure the trimer is the main product other than tetramer and polymer in higher degree, HGH with R1 can be added in excess (e.g. 10 folds more), or one of the R1 can be protected/blocked before the coupling.

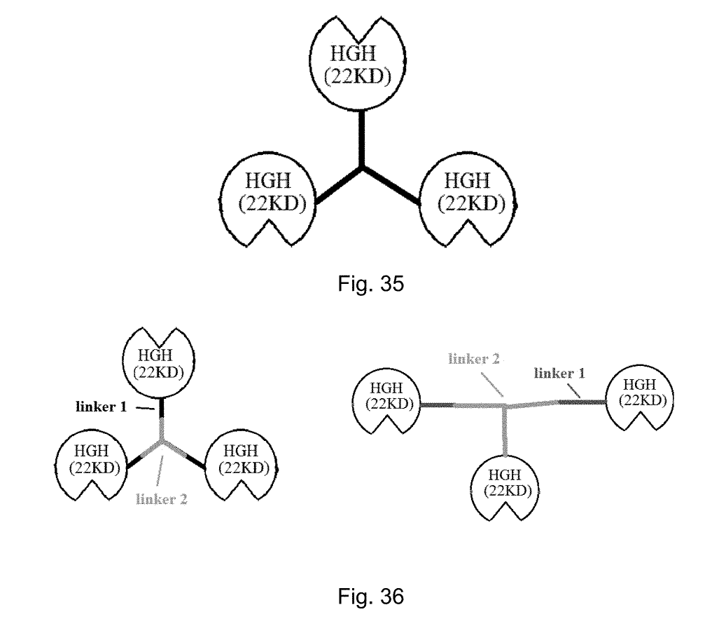

The trimer can also be constructed with a linker having three arms as shown in FIG. 35. For example, the 3 arm linker can be a three arm PEG or a three arm hydrophilic peptide (e.g. peptide rich of Ser, Thr, Glu, Asp) or their conjugate having a MW between 2K˜20 KD. In another example (FIG. 36), linker 1 and liker 2 are connected covalently. Linker 2 and linker 1 are conjugated to HGH (to its Gln) with MTgase and then coupled together using the reactive group on linker 1 and liker 2. Linker 1 and 2 can be functionalized PEG having a MW between 500˜10 KD.

Alternatively, extended in vivo half life of pharmaceutically active protein can be achieved by cross linking the protein non-covalently with linker having multiple affinity group (e.g. antibody or its fragment such as Fab, aptamer or an affinity peptide that can be generated using phase display or the method similar to the development of masking peptide used in probody or screening or rational design) for the protein. Optionally the linker is biodegradable (e.g. an enzyme cleavable peptide). The affinity group can bind with the protein at its active site or non active site.

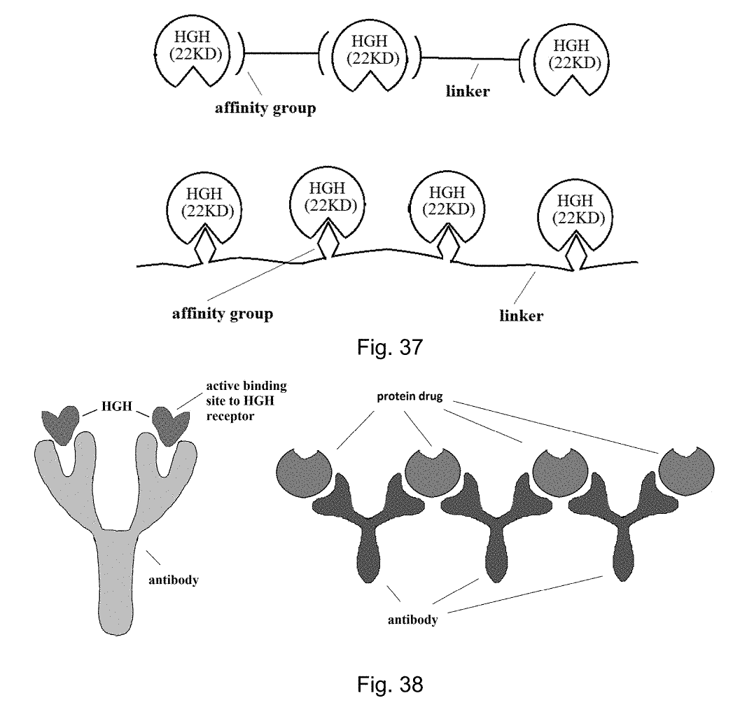

FIG. 37 illustrates two formats to crosslink HGH to extend its in vivo half life. One format is to use a linker having affinity groups binding to HGH's non-receptor binding site at both ends to crosslink HGH. In one example, the affinity group is a 30 AA (amino acid) peptide and the linker is a peptide having 10 AA or a short PEG. Another format is to have a linker carrying multiple affinity groups binding to HGH's receptor binding site.

The linker having multiple affinity groups can be a protein or a peptide having multiple affinity groups, e.g. an antibody, since each antibody has two binding sites. The binding site for the affinity groups can also be introduced artificially to the pharmaceutically active protein. For example, biotins can be attached to the target protein by expression or chemical conjugation and avidin can be used to crosslink the said biotinylated protein for longer in vivo half life. In some examples, the protein is modified with Thermo Scientific EZ-Link Sulfo-NHS-Biotinylation Kit (#21425) or EZ-Link Pentylamine-Biotin (#21345) using the provided protocol from the vendor and then dialyzed to remove the uncoupled. Next avidin or streptavidin is added to the biotinylated protein at 1:2 ratio in PBS for 30 min to form the binding complex, which will have longer in vivo half life compared with the original protein.

Another format is to use protein specific antibody or antibody fragments or aptamer to form an immuno complex or aptamer-protein complex, which will have higher molecular weight (may also protect the protein from enzyme degradation) therefore slower elimination. The binding of antibody/aptamer can be either targeting the protein's active site or non active site. In one example, antibody against HGH's non binding region is mixed with HGH at 1:2 ratio to form its immuno complex, this complex can be used as therapeutics having extended half life to be administrated to the patient. It can also be two antibodies binding with one protein format (the sandwich type binding format similar to those seen in ELISA). Optionally the protein binding with antibody does not activate complement, which can be archived by engineering the antibody. Mutation can be introduced to the antibody FC to remove complement binding (e.g. to c1q), binding to FcγR as well as binding to CR1. FIG. 38 shows two examples using the strategy described above. Bispecific antibody that binds to two different epitopes of the target protein can be used to crosslink the protein.

Alternatively, two antibodies targeting two different epitopes can be connected together (e.g. by fusion or conjugation) to act as a bispecific antibody to cross link target proteins. One example of this kind of two antibody conjugate is shown in FIG. 5 and FIG. 6. Antibodies or antibody fragments targeting different epitope of the protein (e.g. HGH) can be screened to obtain the antibody/antibody fragment providing the best potency and pharmacokinetic property (e.g. in vivo half life).

In some embodiments, antibody fragment containing the epitope binding region is used to form the immuno complex to extend the half life of protein. Suitable antibody fragment can be selected from F(ab′)2 (110 KD), Fab′ (55 KD) Fab (50 KD) Fv (25 KD) which can be cross-linked to improve its stability, scFV, di-scFV, sdAb or the like. In one example, Fab or half-IgG (rIgG) against HGH can be mixed with HGH at 1:1 ratio to form the immuno complex, which can be used as a controlled release HGH drug. Different Fab (e.g. Fab bind with different region of HGH) can be screened to achieve the desired in vivo stability. The resulting binding complex has a MW>70K therefore the kidney clearance rate is reduced. The MW of Fab (50K) ensures that it will have similar clearance rate as HGH therefore reduce the buildup of Fab against HGH.

Optionally the antibody or antibody fragment including FC fusion protein used in the current application can engineered/mutated on the FC to remove complement binding (e.g. to clq), binding to FcγR as well as binding to CR1. The Fc region can also be engineered/mutated to adjust its FcRN binding capability (e.g. provide higher binding affinity for longer Fc containing protein in vivo half life).

The current invention discloses methods for Protein drug half-life extension with Protein Drug dimer, trimer (or higher degree oligomer) using protein as monomer building block. Many small therapeutic proteins (e.g. 10-30 KD) require high MW PEG to reduce rapid renal clearance (>60 KD). High MW PEG may cause cell vacuolation, reduced protein activity, solubility issues and high viscosity; and mono-PEGylation may not provide enough protection against protease/peptidase. The current invention discloses Protein dimerization or trimerization (or higher degree oligomer) for half life extension.

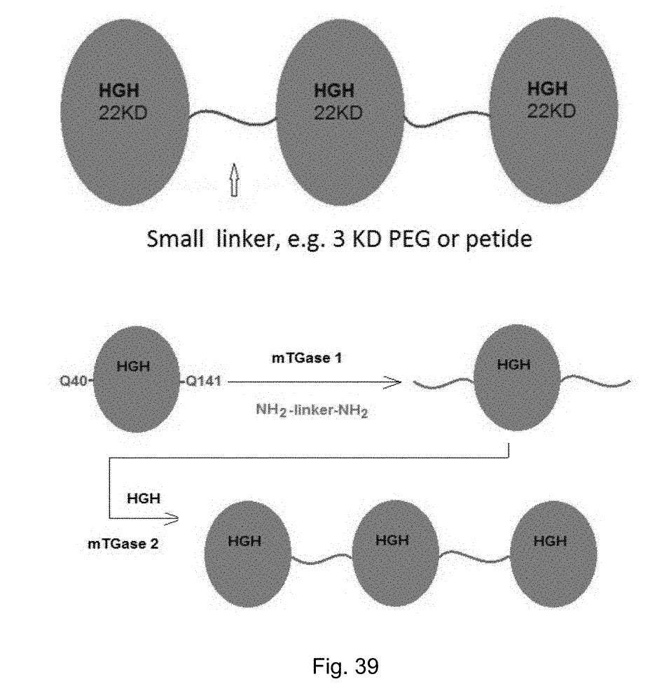

FIG. 39 shows examples of PEGylated HGH (Human Growth hormone) trimer for half-life extension using a small size PEG (or peptide) as linker and an example of its synthesis. The HGH suitable for the current invention can be HGH (Somatropin) from pituitary origin (191 amino acids, the SEQ ID No.1 disclosed in U.S. Pat. No. 8,841,249) having Accession Number: DB00052 (BIOD00086, BTD00086). For example, a low MW PEG (e.g. its MW can be a number between 5K˜25K) having —NH2 groups at its two ends can be used as a linker, alternatively, a peptide having 30˜200 amino acid residuals and two —NH2 groups at it two ends can also be used. The conjugation can be performed using transglutaminase (TGase) to couple the linker to the glutamine in the HGH. Preferably, the linker is introduced at the positions corresponding to positions glutamine 40 and/or glutamine 141 in HGH. The use of transglutaminase (TGase), and in particular microbial transglutaminase (mTGase) from Streptoverticillium mobaraenae or Streptomyces lydicus allows a selective introduction of the linker at positions 40 and/or 141, and the remaining 11 glutamine residues are left untouched despite the fact that glutamine is a substrate for transglutaminase. The protocol of MTGase can be found in many publications such as U.S. Pat. No. 8,841,249 and can be readily adopted for the current application. In the example shown in FIG. 39, excess linker (e.g. di-amino PEG at 10˜20 folds excess to the HGH amount) is added to the HGH and the coupling is performed with mTGase. The resulting HGH carrying two linkers on each HGH monomer is purified to remove unconjugated linker and unconjugated/mono conjugated HGH. Next excess amount of unconjugated HGH (e.g. 20 folds excess) is mixed with the previously prepared di-conjugated HGH and the coupling is performed with mTGase. The resulting conjugate is the HGH trimer having two linkers in the middle HGH and one linker on each end HGH. Using special mTgase can allow the site specific conjugation at either glutamine 40 or 141 or both.

For example, the use of a transglutaminase to attach PEG to HGH on glutamine residues has previously been described in U.S. Ser. No. 13/318,865 and U.S. Ser. No. 12/527,451. The method may be used in accordance with the present invention for attachment of the linker and linker conjugated with HGH. The TGase used can be microbial transglutaminase according to U.S. Pat. No. 5,156,956. In one embodiment, a hGH is dissolved in triethanol amine buffer (20 mM, pH 8.5, 40% v/v ethylene glycol). This solution is mixed with a solution of amine donor linker, e.g. NH2-PEG-NH2 dissolved in triethanol amine buffer (200 mM, pH 8.5, 40% v/v ethylene glycol, pH adjusted to 8.6 with dilute hydrochloric acid after dissolution of the amine donor). Finally a solution of mTGase (˜0.5-7 mg/g hGH) dissolved in 20 mM PB, pH 6.0 is added and the volume is adjusted to reach 5-15 mg/ml hGH (20 mM, pH 8.5). The combined mixtures are incubated for 1-25 hours at room temperature. The reaction mixture is monitored with by CIE HPLC. The resulting HGH having two linkers on each protein is purified.

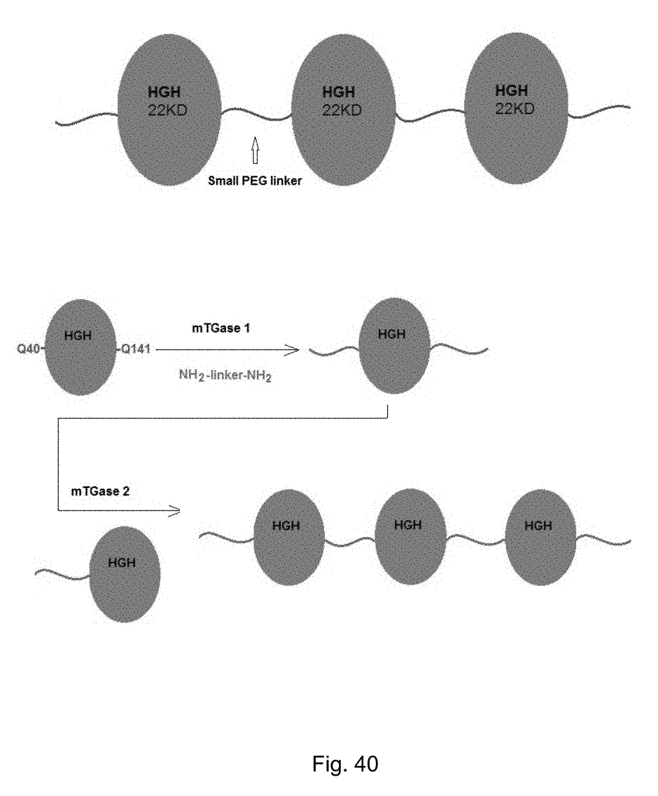

Alternatively, if excess amount of mono-conjugated HGH (e.g. 20 folds excess) is mixed with the previously prepared di-conjugated HGH and the coupling is performed with mTGase. The resulting conjugate is the HGH trimer with two linkers on all HGH (FIG. 40). In some embodiment, the linker for preparing the mono-conjugated HGH has one end with —NH2 group and another end without —NH2 group. By using special mTgase having different substrate specificity and altering the conjugation sequence and ratio, different trimer or oligomer can be prepared readily by skilled in the art.

Other site specific conjugation method can also be used to construct the oligomer. It could be as chemo selective synthesis such as click chemistry, thiol maleimide coupling and etc. It can lso be enzyme based coupling other than mTgase conjugation, such as sortases based conjugation as well as the combination of different conjugation method. Sortase, particularly sortase A from S. aureus, has been recognized for some time as a useful protein engineering tool, allowing the ligation of oligo-glycine-containing polypeptides or small molecules to proteins containing a sortase-penta-peptide motif, LPETG (SEQ ID NO: 9) in case of S. aureus sortase A, (LPETG: Leu-Pro-Glu-Thr-Gly), e.g.: -LPETGG (SEQ ID NO: 10)+GGGGG-(SEQ ID NO: 11)→-LPETGGGGG-(SEQ ID NO: 12). The Glu (E) in the sortase-penta-peptide motif can be replaced with other amino acid, which is fully disclosed in the literature and patents. The protocol of sortase based conjugation can be found in many publications (e.g. U.S. patent application Ser. No. 14/774,986) and can be readily adopted for the current application.

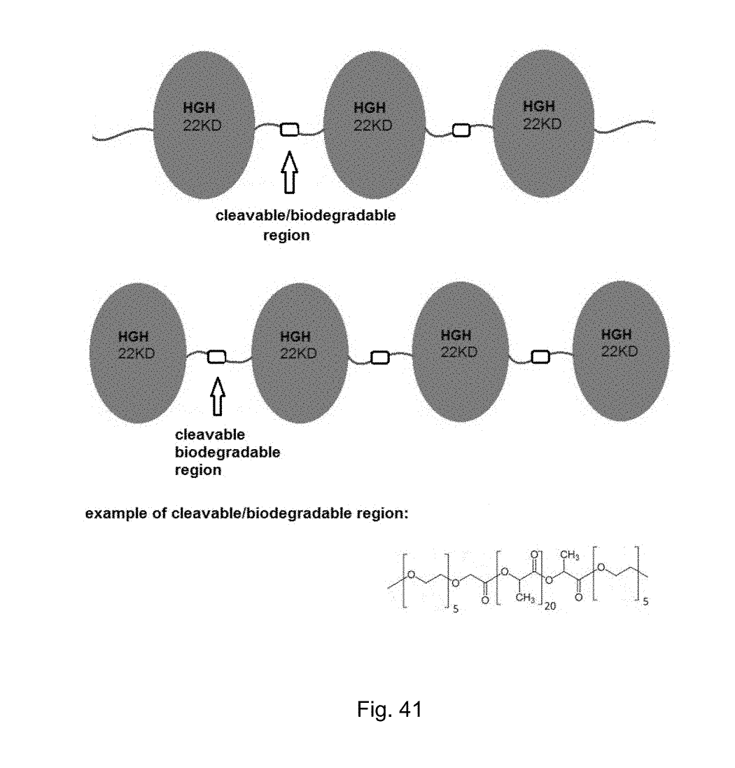

The linker used to construct protein oligomer (e.g. dimer or trimer) can also contain one or more cleavable/biodegradable region (FIG. 41), which is essentially a cleavable/biodegradable linker similar to that previously described. This will allow the release of protein monomer or lower degree oligomer slowly in vivo and therefore provide better control on in vivo stability.

This method will reduce renal clearance efficiently with minimal linker (e.g. PEG) content. Small PEG can be used (e.g. 1˜15 KD) to achieve total MW of the conjugate >60K to avoid problems associated with high MW PEG, linear structure also increase hydrodynamic size. It can offer better protection against protease degradation. The resulting more drug load and higher activity than mono-pegylated protein due to multivalency will reduce drug amount and volume to improve the comfort of subcutaneous injection. It will provide defined structure and allow site specific conjugation. Higher degree than trimer (e.g. tetramer), biodegradable linker and non-PEG linker (PVA linker, peptide based linker and etc.) can be readily adopted. It is suitable for many proteins with MW 10˜30K. Examples of the protein can be found in well known publications and prior arts, include but not limited EPO, IFN-α, IFN-β, IFN-γ, factor VIII, factor IX, IL-1, IL-2, insulin, insulin analogues, granulocyte colony stimulating factor (GCSF), fibrinogen, thrombopoietin (TPO) and growth hormone releasing hormone (GHRH).

The protein dimer, trimer, tetramer or higher degree oligomer can also be produced by expression as recombinant protein, in which each monomer is connected by a flexible peptide linking region from the one's C terminal to another's N terminal. The protein dimer, trimer, tetramer or multimer drug is expressed as a whole protein having several monomeric units connected by hydrophilic peptide linking regions, e.g. Asp, Glu, Ser/Gly/Ala rich peptide having 20˜200 AA (amino acids), the negative charged Asp/Glu can inhibit the endocytosis of the protein drug by the cell to reduce receptor mediated clearance, optional protease cleavable sequence can be incorporated into the linking region to adjust its PK. In some embodiments the peptide linker suitable for the current invention contains 10˜150 AA; preferably between 15˜200AA; the sum of glycine (G), alanine (A), serine (S), threonine (T), glutamate (E), aspartate (D), and proline (P) residues constitutes more than about 90% of the total amino acid residues of linker; the sum of glutamate (E) and aspartate (D) residues constitutes more than about 20% of the total amino acid residues of linker. In some embodiments preferably the sum of glutamate (E) and aspartate (D) residues constitutes more than about 30% of the total amino acid residues of linker. Preferably the linker is flexible and displays a random secondary/tertiary structure. Optionally the linker comprises one or more a cleavage sequence (e.g. peptidase/protease cleavage sequence).

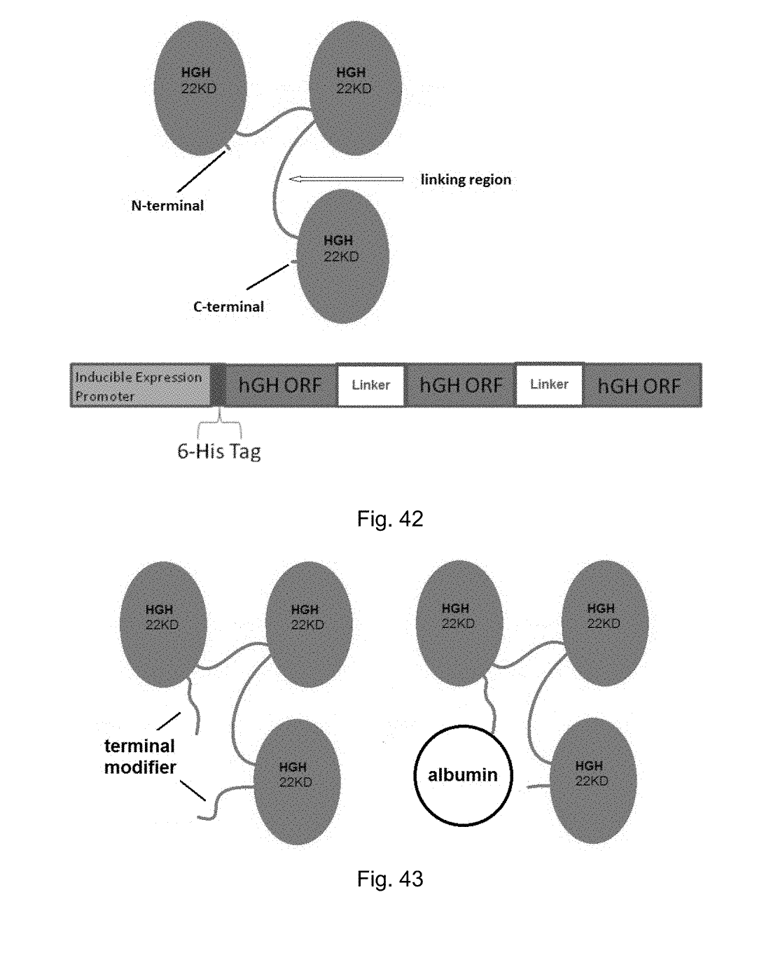

Preferably the linker constitutes less than about 50% of the total amino acid residues of resulting oligomer. In some embodiments more preferably the linker constitutes less than about 40% of the total amino acid residues of resulting oligomer. In some embodiments more preferably the linker constitutes less than about 30% of the total amino acid residues of resulting oligomer. Preferably the resulting oligomer has a MW>60K. An example of the linker is -GG(ASEGSDEAEGSEASGEGDG)₅-GG (SEQ ID NO: 4). FIG. 42 shows an example of a recombinant HGH trimer and its construction. It can be prepared with CHO cell or E coli expression construct. The Human Growth Hormone Trimer with linker sequence use HGH/Somatropin cDNA identical to HGH from pituitary origin (191 amino acids) Accession Number: DB00052 (BIOD00086, BTD00086). It is tagged with 6-His or other motif for purification. The peptide linker is -GGD(GSEGSEGEASEGSAEGEG)₂-DGG-(SEQ ID NO: 5). The protocol of recombinant protein expression is well known to the skilled in the art and protocols from the publications can be readily adopted for the current invention.

N terminal or C terminal modifier can also be introduced to the oligomer to the N terminal and/or C terminal of the oligomer by recombinant technology. Antibody FC or albumin can also be expressed together with the above oligomer. For example, they can be attached to the N terminal or C terminal of the oligomer by recombinant technology. N terminal and/or C terminal of the oligomer can also be added with modifier sequence such as a flexible peptide sequence similar to the linker using recombinant technology to adjust its in vivo half life (FIG. 43). The alkyl/fatty acid conjugation can also be employed. The protein oligomer generated from recombinant expression can also be further conjugated with half life modifier (e.g. PEG) with site specific conjugation method (e.g. sortase or mTgase conjugation).

Besides trimer, protein drug monomer or dimer with optional terminal half-life modifier can also be used to increase their half-life. The terminal half-life modifier can be Fc or albumin or alkyl/fatty acid or sphingolipids or cholesterol derivative (e.g. 3β-cholesterylamine). The key is to use a flexible linker to separate the Fc or albumin with protein drug monomer with enough distance and to separate the protein monomer themselves with enough distance if multiple protein monomer is incorporated within. This will reduce the immunogenicity and increase the size of the whole drug as well. In some embodiments, the flexible linker can be PEG (e.g. MW between 5K˜20K) or a flexible peptide linker (e.g. between 40˜200AA) such as those described before or similar to those used in Xten from Amunix or PAS linker (proline-alanine-serine polymer from XL-Protein GmbH). Examples of these kind of construct are shown in FIG. 44, where HGH is the protein and each HGH contains two flexible linkers (e.g. at its N and C terminal by recombinant technology or by site specific conjugation using PEG).

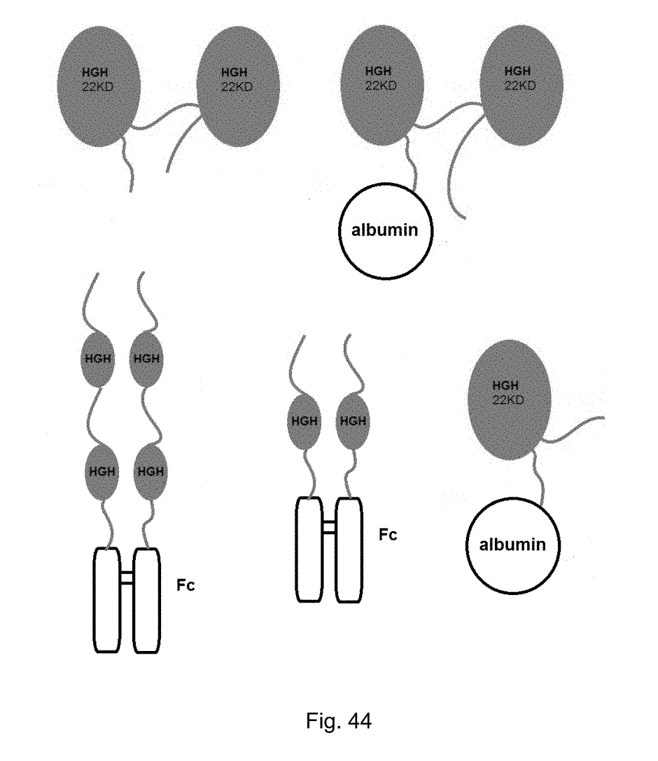

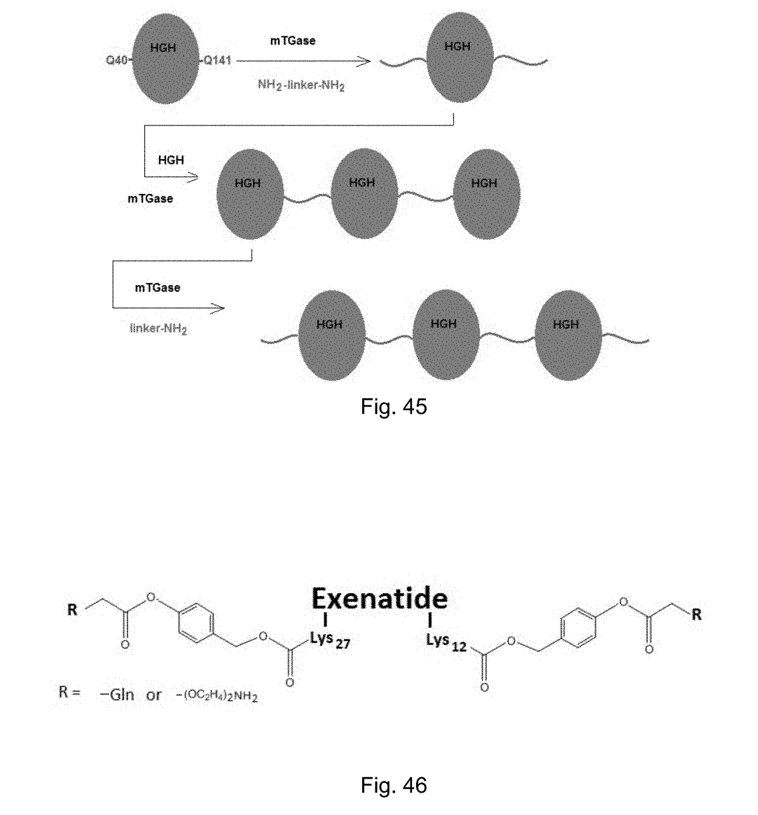

FIG. 45 shows another example of the synthesis of HGH trimer. In case PEG is used as linker, the mTgase (microbial transglutaminase) conjugates amine groups of the PEG to the Gln of the HGH site specifically. In step 1, excess NH2-PEG-NH2 (>20 folds of HGH, MW between 5K˜20K) is used to produce HGH with two PEG. In step 2 the resulting HGH having two PEG each having one —NH2 terminal react with excess free HGH to generate the trimer. In step 3, the trimer is further conjugated with mono amine PEG (>20 folds, MW between 5K˜20K) to get the final product. Gel filtration column or HIC column or ion exchange column can be used for purification. For example, HGH is dissolved in borate buffer (20 mM, pH 8.5). This solution is mixed with a solution of amine donor linker, e.g. NH2-PEG-NH2 dissolved in borate buffer (200 mM, pH 8.5, 20% v/v ethylene glycol, pH adjusted to 8.6 with dilute hydrochloric acid after dissolution of the amine donor). Finally, a solution of mTGase (˜0.5-1 mg/g hGH) dissolved in 1× mM PBS is added and the volume is adjusted to reach 5-15 mg/ml hGH (20 mM, pH 8.5). The combined mixtures are incubated for 10-20 hours at room temperature. The reaction mixture is monitored with by CIEX HPLC or RP-HPLC. The linker is introduced at the positions corresponding to positions glutamine 40 and/or glutamine 141 in HGH. The resulting HGH is purified. The resulting HGH having two PEG modification from step 1 can also be used for HGH half-life extension. In this case the PEG used for HGH modification can only have one amine end, preferably having a MW between 10K-30K. Instead of amine another end of the PEG can be —COOH or —OH or methyl group or conjugated with alkyl/fatty acid or sphingolipids or cholesterol derivative (e.g. 3β-cholesterylamine). One of the PEG conjugation can also be performed based on amide bond formation between PEG and HGH. For example, the first PEG (e.g. MW=15K) is conjugated to the N terminal of HGH using PEG-NETS ester or PEG-CHO followed by reduction with NaCNBH3; and the second PEG (e.g. MW=20K) is conjugated to Gln 141 with mTGase and mono amino PEG. Alternatively, the C terminal or N terminal of HGH or both can be added a flexible peptide linker (e.g. 50AA-200AA) by expression and next a PEG (e.g. MW=20K) is conjugated to Q141 of HGH. In another example, the C terminal of HGH is added a flexible peptide linker (e.g. 50AA-200AA) and next a PEG (e.g. MW=20K) is chemically conjugated to the N terminal of HGH.

The protein oligomer can also be constructed with the combination of recombinant technology and site specific conjugation. First the protein monomer having reactive N terminal and/or C terminal peptide end can be constructed with recombinant technology. Next the reactive N terminal and/or C terminal peptide end can be used as linking region to conjugate with other protein or linkers (e.g. peptide or PEG) with site specific conjugation method. For example, the protein monomer can be expressed with reactive end such as Gln/Lys to be used for mTgase based conjugation or LPETG/oligo glycine for sortase based conjugation. Optionally a peptide linker can be added between the native protein and the reactive end during the expression. This strategy can avoid the potential folding issue in direct protein oligomer expression. For example, the N terminal of one HGH is added with oligo glycine during expression and the C terminal of another HGH is added with LPETGG through a flexible peptide linker (e.g. the G/A/D/E rich peptides described above) during expression. Next the two modified HGH monomers are conjugated together with sortase mediated ligation. In another example, a HGH having N terminal oligo glycine and C terminal LPETGG (e.g. oligo glycine-peptide linker-HGH-peptide linker-LPETGG) is expressed, next it is used as monomer to prepare oligomer with sortase mediated ligation, the resulting oligomer can be a mixture of HGH oligomer having different degree of polymerization (e.g. dimer, trimer, tetramer and etc.). In another example, excess amount of (e.g. 5˜10 folds) expressed HGH-peptide linker-LPETGG reacts with expressed GGGGG-HGH-peptide linker-LPETGG using sortase mediated ligation to generate HGH-peptide linker-LPET-GGGGG-HGH-peptide linker-LPETGG, which is a HGH dimer. Next the purified HGH dimer is conjugated with GGGGG-HGH using sortase mediated ligation to form the HGH trimer: HGH-peptide linker-LPET-GGGGG-HGH-peptide linker-LPET-GGGGG-HGH. The expressed HGH can also be conjugated with synthetic molecules (e.g. modified PEG) bearing reactive groups for further conjugation and then the resulting HGH is used to construct oligomer. For example, expressed HGH-(G)n-LPETG is conjugated with GGGGGG-PEG-Azide to form the HGH having Azide group with sortase, next the HGH azide is conjugated with a HGH having two alkyne groups (which can be synthesized by coupling alkyne-PEG-NH2 with HGH with mTgase) using click chemistry. The product is a HGH trimer connected with cycloaddtion product of azide with alkyne.

The current inventions also disclose methods for peptide drug half-life extension. One is peptide drug oligomer using peptide as monomer building block. Another is peptide drug conjugated on linear peptide carrier. Peptide drug requires more than trimer/tetramer to get enough MW>60K, which is important to reduce kidney clearance. The current invention uses peptide drug as monomer to prepare oligomer/polymer: -[peptide drug]ₙ-to achieve high MV to prevent renal clearance and enzyme degradation. The monomer contains one or more cleavable linker such as a self immolative linker to allow the release of active drug. Hydrophilic region (e.g. PEG or hydrophilic peptide) can be incorporated to the polymer to improve its solubility.

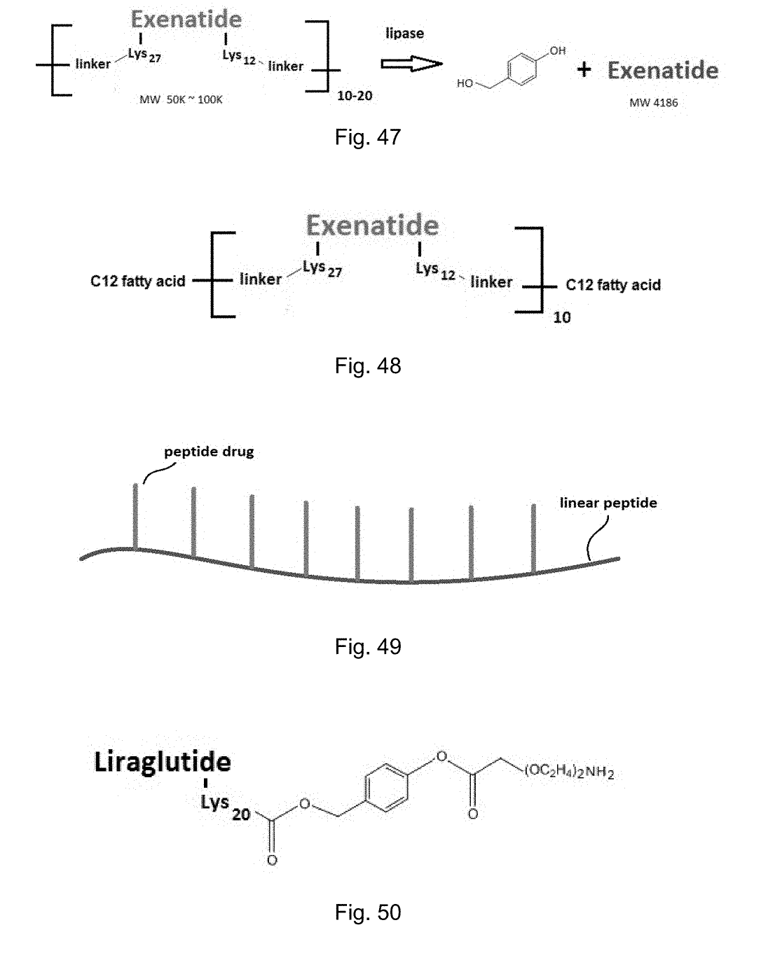

Each peptide drug can be added with two reactive groups as peptide drug monomer for polymerization. For example, FIG. 46 shows an Exenatide monomer. The ε-amines of Lys 27 and Lys 12 in Exenatide (MW 4200) are coupled with Gln or PEG-NH₂ via self-immolative linkers to generate two Exenatide monomers; which allow mTGase polymerize Gln modified monomer with PEG-NH₂ modified monomer. Coupling Gln and PEG-NH₂ to the same Exenatide monomer may simplify the chemistry with the risk of intra molecule conjugation. Other formats such as non-peptide drug monomer can also be used, e.g. using Gln-PEG-Gln and PEG-NH₂ modified Exenatide for polymerization. The resulting polymer can be degraded to release free drug Exenatide (FIG. 47). Amino acid in the peptide interfering polymerization can be protected before polymerization (e.g. protect Gln 13 in Exenatide with Mtt or photo cleavable protection group if replacing Gln affecting its activity). Spacer can be incorporated into the linker to adjust solubility and chemistry. Biodegradable linker (e.g. hydrolysable or enzyme cleavable linker) can be used. Other polymerization chemistry can also be used (e.g. thiol-maleimide coupling, click chemistry) besides enzyme based conjugation. High drug content can be achieved. High degree polymerization can lead to formation of microspheres, which have longer half-life than soluble polymer. Optionally one or more alkyl group such as fatty acid can be conjugated to the monomer or resulting polymer to allow it bind with albumin to further increase its half life (FIG. 48). The alkyl group can also be built in the monomer or linker.

This strategy can be applied to any peptide drug by replacing Exenatide with other peptide drug. The principle is to build monomer with peptide drug by adding reactive groups for polymerization to the peptide and then perform polymerization. The resulting peptide drug polymer will have high MW and steric hindrance therefore reduce its clearance.

Alternatively, peptide drug half-life extension can be achieved with linear peptide carrier. Synthetic polymers (e.g. PVA, PAA and dextrin) were used to conjugate with drugs for controlled release/targeted drug delivery; their polydisperse structure creates hurdle in drug development and regulatory approval. The current invention use site specific conjugation of peptide drug to synthetic linear peptide (structure shown in FIG. 49).

The linear peptide has defined MW, which can be achieved by peptide synthesis (if <70 mer) or expression (if longer peptide is required). The linear peptide is rich of hydrophilic AA and small AA (e.g. Ser, Glu, Ala and Gly) to provide a highly flexible/hydrophilic backbone and avoid the formation of secondary structure. The linear peptide contains either multiple Gln or multiple Lys to provide functional group for mTgase conjugation, preferably >5. For example: polymerized GESGQGSEG (SEQ ID NO: 7) such as [GESGQGSEG] ₂₀ can be used as a linear peptide to conjugate to peptide drug. The peptide drug contains Gln (for lys rich linear peptide) or free —NH2 (for Gln rich linear peptide) to be conjugated to the linear peptide with mTgase directly or via a linker (permanent or cleavable). For example, a self immolative linker can be used to couple the peptide drug to the linear peptide to release the original peptide drug after degradation. The FIG. 50 shows a liraglutide derivative having a cleavable linker (Lys 20 not conjugated with Glu-palmitoyl group). It can be coupled to the said linear peptide with Gln to extend its half-life.

Gln/Lys in the peptide drug that can cause intra molecule conjugation can be protected before mTgase conjugation and deprotected after conjugation. Cleavable regions can also be incorporated into the linear peptide (either peptide based or non-peptide based) to improve peptide drug release.

Non-AA monomer can also be incorporated into the linear peptide. For example: [GESGQGSEG-PEG2000] ₈ can be synthesized easily with Fmoc-PEG2000-COOH and Fmoc-GESGQGSEG-COOH using SPPS, which will provide 8 Gln for peptide drug conjugation and a ˜25K backbone. With 8 Exenatide conjugated to it, the MW will be >60K and may have a even bigger hydrodynamic size. This method will provide monodisperse MW of the conjugate and well defined structure of the conjugate. High drug content (>50% in weight) in the conjugate can be achieved. The synthesis of the conjugate is straightforward and fine tuning of the PK can be achieved readily.

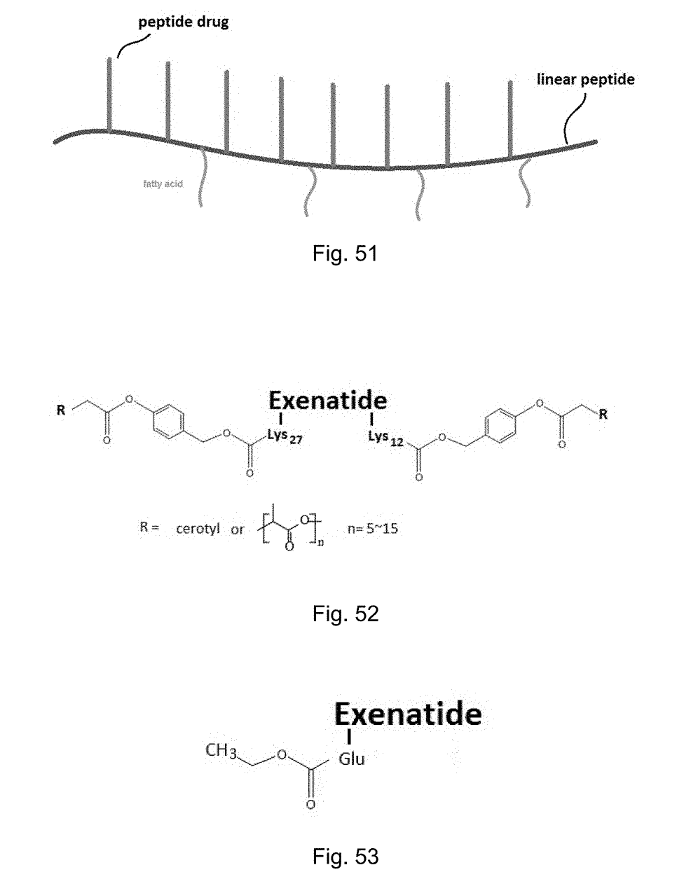

Optionally one or more alkyl group such as fatty acid can be conjugated to the monomer or resulting polymer to allow it bind with albumin to further increase its half life. The alkyl group can also be built in the monomer or linker as shown in FIG. 51. Other lipid type molecule such as Sphingolipids or Cholesterol derivative (e.g. 3β-cholesterylamine) can also be used instead of fatty acid.

The current invention also disclose a method to decrease the solubility of the drug to make it has a low solubility so it will be in the form of micro particles in vivo, therefore has extended half-life. The principle is to conjugate one or more lipophilic molecules (such as a long alkly chain or a short poly lactic acid chain) to the drug via cleavable linker such as self-immolative linker.

One example is shown in FIG. 52. Another Example is shown in FIG. 53, in which 5 Glu in Exenatide is esterized with alkyl alcohol. The insoluble drug can be formulated as liposome or suspension to be injected. Other lipid type molecule such as Sphingolipids or Cholesterol derivative (e.g. 3β-cholesterylamine) can also be used instead of fatty acid.

The current invention also discloses a method for protein or peptide or small molecule drug half life extension using drug- self immolative linker-half life modifier conjugate. The formula below shows the general structure of the drug-self immolative linker-half life modifier conjugate.

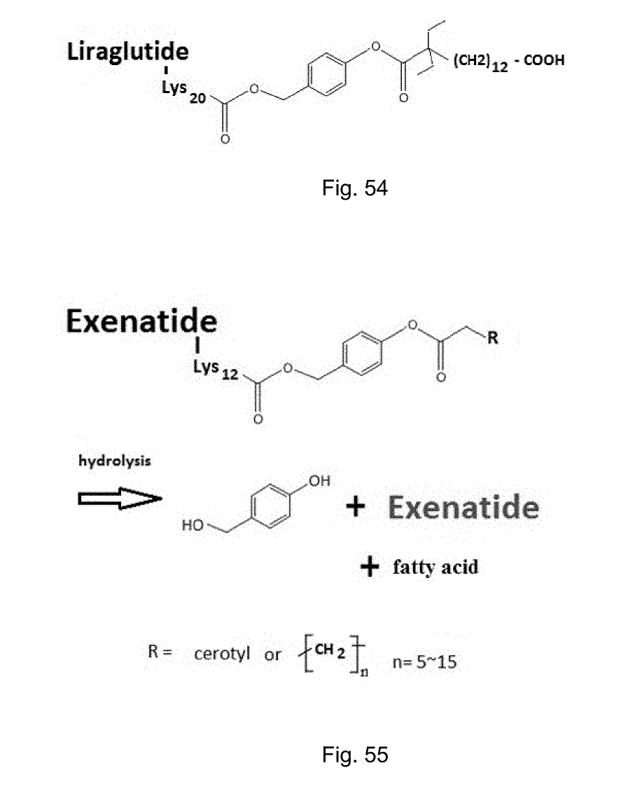

The drug (or drug multimer) is conjugated to a self immolative linker; the self immolative linker is also conjugated to a half life modifier. Examples of drugs include small molecule drug, peptide drug and protein drug. The drug can be conjugated to self immolative linker with its amine or —COOH or —OH or —SH group. Example of half life modifier include albumin binding molecule (e.g. fatty acid, long alkyl chain, small molecule or peptide or aptamer having high affinity to albumin), sphingolipids or cholesterol derivative such as 3β-cholesterylamine, antigen, FcRn binding molecule, PEG, FC of the antibody, polypeptide having large MW and etc. The half life modifier can be either in monomer form or oligomer form. The cleavage of self immolative linker will release the original drug in vivo, which preferably is the active drug. Other cleavable linkers such as those in U.S. patent application Ser. No. 12/865,693, U.S. Ser. No. 12/990,101 and U.S. Ser. No. 09/842,976 can also be used. The cleavable (e.g. hydrolytic) site of the linker can be adjusted (e.g. adding steric hindrance) to control its cleavage rate in vivo. One example (FIG. 54) shows a liraglutide conjugated with a self immolative linker and a fatty acid to bind with albumin to increase its half life in vivo. One or more hydrophilic region/modifier (e.g. PEG or hydrophilic peptide) can be incorporated into the conjugate to improve its solubility.

Another example (FIG. 55) shows exenatide conjugated with a self immolative linker and an alkyl chain to bind with albumin to increase its half life in vivo, which release the active drug in vivo.

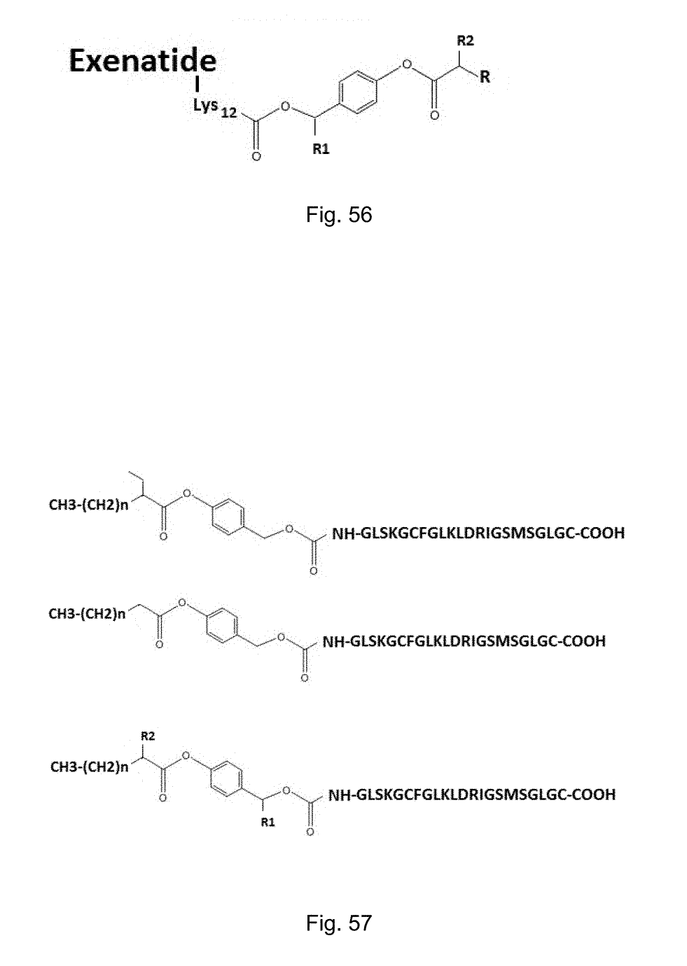

The hydrolytic rate of the linker can be adjusted by incorporating functional group into the linker (e.g. bulky R1, R2 in the FIG. 56) to adjust its stability.

Another example is shown in FIG. 57 involving C-Type Natriuretic Peptide: NH2-GLSKGCFGLKLDRIGSMSGLGC-COOH [native CNP; CNP22] (SEQ ID NO: 8). In FIG. 57 CNP peptide is conjugated to an alkyl chain with a self immolative linker, where n=5˜20 and R1, R2 are bulky group to provide steric hindrance or electron donating/withdrawing group to adjust the ester bond stability.

The drug can also be in the multimeric format (formula below) connected by cleavable linkers (e.g. self immolative linker).

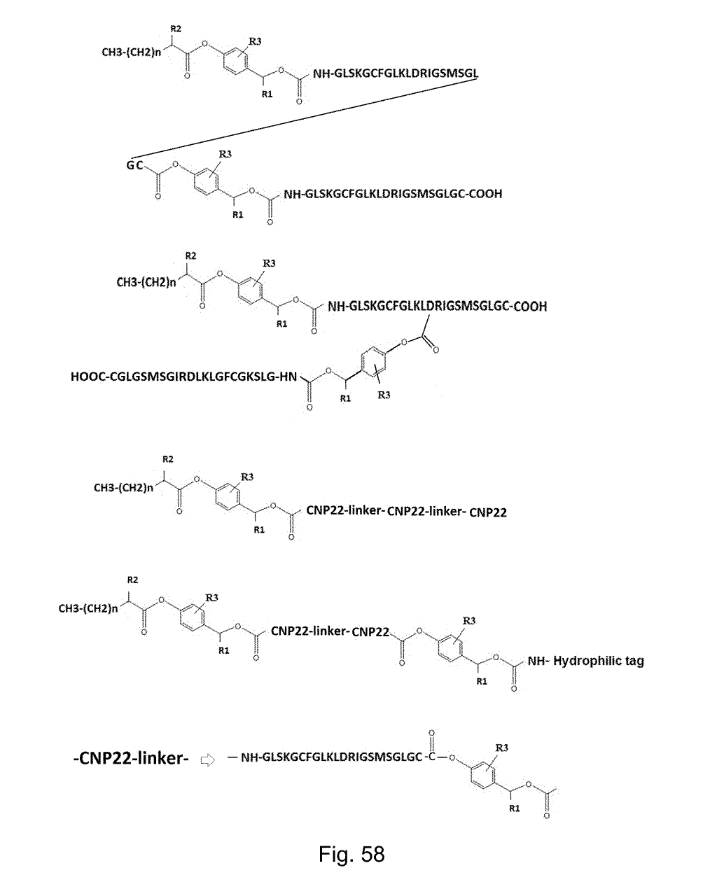

For example, as shown in the examples in FIGS. 58, R1, R2 and R3 are bulky group (e.g. tert-butyl group) to provide steric hindrance or electron donating/withdrawing group to adjust the ester bond stability, two C-Type Natriuretic Peptides are conjugated together using ester linkage via their C terminal or the —COOH of D (Asp) to another's N terminal and then conjugated to a fatty acid via an ester linkage.

Example of hydrophilic tag includes PEG or hydrophilic peptide (e.g. E, D, S rich peptide) to increase the solubility of the conjugate. Other C-Type Natriuretic Peptide analogues/derivatives/mimic can also be used instead of the native—Type Natriuretic Peptide, such as those described in J Pharmacol Exp Ther. 2015 April; 353(1):132-49.

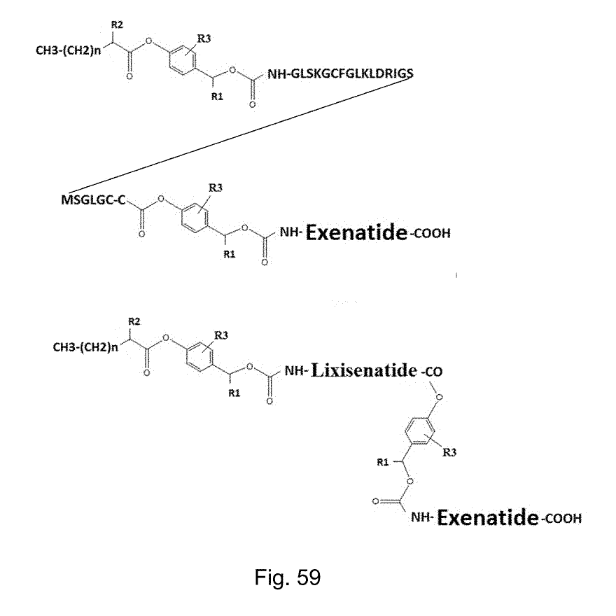

The multimeric drug is not limited to homo oligomer/polymer, it can also be the conjugate of two or different drugs (hetero oligomer/polymer) of the same biological function or different biological functions. Examples can be found in FIG. 59, where the multimeric drug contains both CNP-22 and Extennatide.

The current invention also provide methods to treat cancer especially to prevent tumor metastasis and tumor recurrence by removing and/or inactivating (e.g. killing) the circulating tumor cells (CTC, both single CTC cells and CTC aggregates) in the blood after removing the tumor or treating the tumor with therapeutical means such as surgery, chemotherapy, radiation therapy, photodynamic therapy, photon radiation therapy, laser therapy, microwave therapy, ultrasound, cryogenic therapy, heat therapy or combinations of them. In some embodiments, the therapeutical means targets the primary tumor. The method to prevent tumor metastasis and tumor recurrence in the current invention comprises two steps 1) removing the tumor or treating the tumor with therapeutical means such as surgery, chemotherapy, radiation therapy, photodynamic therapy, photon radiation therapy, laser therapy, microwave therapy, cryogenic therapy, heat therapy or combinations of them; next 2) removing the circulating tumor cells from the blood and/or inactivating the circulating tumor cells by extracorporeally circulating blood.

In some embodiments, the CTC amount in the blood of the patient is counted before the surgery or tumor treatment (e.g. radiation or chemotherapy), and then the CTC amount in the blood of the patient is counted during and/or after the treatment, if increase is observed (e.g. >50%), the patient is treated with CTC removal/inactivating by extracorporeally circulating blood.

In general, these circulating tumor cells are removed(inactivated) by blood purification (e.g. hemopurification) of extracorporeally circulating blood through a blood purifier that can remove/kill the circulating tumor cells in the blood and/or inactivate the CTC while it is outside the body by extracorporeally circulating blood. What passes the blood purifier or what is treated with CTC inactivation means can be either whole blood or the blood component containing the CTC. The methods are described in U.S. patent application Ser. No. 13/444,201 as well as PCT application PCT/US12/33153. Hemopurifier and blood dialysis device are widely used for many disease such as kidney failure. For example, a solid phase adsorbent that has affinity to the tumor cells can be placed in the blood purifier for the blood purification. For example, the solid phase adsorbent (e.g.

column, filter, fiber, membrane, particle) coated with affinity molecules that can selectively bind with the tumor cells can be used in the blood purification device to remove these cells. Preferably, these affinity molecules have no or low affinity to majority of other normal blood cells.

Cancer cells usually clump together for metastasis. Size based filtration can be used to remove the clumped cancer cells in the blood. These cell clumps (CTC aggregate) are bigger than blood cell size, therefore using a filter that can remove the clumped tumor cells but not the blood cells (such as filter with suitable pore size, e.g. 20 um) for blood purification during or after the surgery can also reduce the risk of metastasis. They can be also be removed by centrifuging the extracorporeally circulating blood as the CTC aggregate will be separated with other cells during centrifugation (e.g. precipitate faster).

In one example, the patient first undergoes a surgery to remove the tumor, either during the surgery or 2 h after the surgery or after one day the blood purification is performed to remove the CTC. First the extracorporeally circulating path is established, the blood comes out from the artery of the patient goes into the blood inlet of the blood purifier and pass through a membrane filter inside the blood purifier and then goes out from the blood outlet and infuse back to the vein of the patient. The filter has a pore size of 20 um and the diameter is 20 cm. The CTC aggregate will be retained on the filter while other cells will pass through. The blood flow rate is 100 ml/min and the operation last for 2 hours. The filter can also be of hollow fiber type similar to those described in FIG. 3˜6 and related examples in the said applications except the pore size is bigger than most single cells but smaller than most CTC aggregate (e.g. pore size 20 um˜30 um). This type of filter can also be used in combination with other CTC removal devices/methods described in the said applications to further remove the single CTC in the blood. For example, the extracorporeally circulating blood of the patient first passes through a 25 um filter to remove the CTC aggregate and then passes through another affinity sorbent type CTC removal device described in the said applications and then goes back to the patient.

The methods and devices for CTC removal described in the previous U.S. application Ser. No. 13/444,201 are to remove CTC from blood. The term CTC includes both single CTC and CTC aggregate.

Another method to remove CTC is to use blood cell separator. When the blood is processed with blood cell separator, most CTC will stay within the leukocyte component in many cases. In some cases CTC will be in the mononuclear cells component and in some cases the CTC will stay in the monocyte portion depending on the cell separator type, its parameter and the nature of the CTC cells (the exact distribution of CTC can be determined experimentally by testing a small amount of blood from the patient). One can readily isolate these components using blood cell separator. Next the portion containing the CTC (e.g. the monocyte portion or the mononuclear cell portion or the entire leukocyte portion) is given the CTC removal/inactivation treatment either continually or in a batch format. Other blood components can be sent back to the body directly after the separation or be combined with the blood component being treated then return to the body. Optionally the other blood components can also pass through a different blood purifier or being treated with CTC inactivating means before going back to the body. The CTC containing leucocytes can also be treated with centrifugation based device again (and optionally be added with buffer/liquid) to further enrich the CTC and remove the healthy cell (e.g. platelet) before go to the next treatment. Because single CTC cells and CTC aggregates may have different property (e.g. size, density which may cause different distribution during centrifugation) so they may stay in different cell layers/portion in blood cell separator. For example, in some patients their single CTCs maybe with white blood cell but CTC aggregates may be in another layer after centrifugation (e.g. at the bottom layer) so the removal of CTC need to be done for both layer/portion of cells. It is preferred to test the patient's blood in a small volume sample using the blood separator or a miniature device that can mimic the blood separator to be used to determine the distribution of the single CTC and CTC aggregates in the cell separation process and use the said distribution to guide the removal of single CTC and CTC aggregates from the patient in the real treatment using blood cell separator. The small volume blood test can also be used to optimize the parameter used for the blood separator to achieve the best CTC removal efficacy. For example, 20 ml of blood is taken from the patient and then processed with a miniature device that mimics the blood separator (e.g. a small centrifuge), multiple cell portion/fraction/ layers are obtained (e.g. divide into 10 fraction/layers based on their sedimentation rate) and each fraction/layer is tested for single CTCs and CTC aggregate count. The fraction/layers having high CTC count will be selected as fraction/portion to be removed. Next the parameter and protocol is transferred to the full size blood cell separator for the extracorporeally circulating treatment and the corresponding cell fractions/portions are removed from the blood, which contains single CTCs and CTC aggregate. The CTC s containing blood cell fraction/portion can be discarded or be treated with other CTC removal (e.g. a CTC purifier using a filter or CTC absorbent)/inactivating means to remove the CTCs, resulting in clean blood part and then return the cleaned part back to the patient. During the process of using blood cell separator for leucocytes, the CTC are with the separated leucocytes and the concentration of CTC and leucocytes are high in that fraction, which allow the leucocytes to in close contact with CTC and boost the immune reaction of the leucocytes against CTC. The fraction can be incubated for a while outside the patient to increase the leucocyte activity against CTC and its source cancer cells.

The current invention described several methods/devices to remove/inactivate CTC. These means can be used independently or in any combination if they are compatible as well as be repeated in one treatment session. For example, the whole blood can first be treated with a centrifugation type blood cell separator and the CTC containing leucocytes and CTC aggregate containing cell portion is sent to an affinity capture adsorbent based purifier or a filtration based separator. After filtration the blocked CTC/other cells (e.g. leucocytes) can be discarded or pass through an affinity capture based purifier or a CTC inactivating device before return to the patient. In another example, the whole blood first pass through a filtration type CTC removing device and the blocked CTC/other cells then pass through an affinity capture based purifier or a CTC inactivating device (or being treated with CTC inactivating means) before return to the patient. In a third example, the whole blood first passes through a filtration type CTC removing device and the blocked CTC/other cells then are sent to a centrifugation type blood cell separator. The resulting enriched CTC containing component can be discarded or be further treated with other type CTC removing/inactivating device/devices/means before return to the patient. At any stage, the resulting blood component containing no or only small number of CTC can be send back to the patient or optionally be treated with another type of CTC removing/inactivating device/means before return to the patient if this small number of CTC also need to be removed. In another example, the whole blood can first be treated with a centrifugation type blood cell separator and the CTC containing leucocytes and CTC aggregate containing cell fraction/portion is sent to a 20 um pore size filter to remove the CTC aggregate and then pass through a column type affinity capture based purifier and then the cleaned blood component is returned back to the patient. In another example, the whole blood can first be treated with a centrifugation type blood cell separator and the CTC containing leucocytes and CTC aggregate containing cell portion is sent to a 25 um pore size filter to remove the CTC aggregate and then mixed with magnetic particles that has specific affinity to CTC and then remove the CTC bound magnetic particles with magnet and then the cleaned blood component is returned back to the patient.

The previous patent applications also disclose method to improve the therapeutic efficacy of medicine by removing the substance in the blood that can bind with the medicine with high affinity using blood purification. There are many medicines take effect by bind with the surface marker of pathogens or human cells. Examples of these kinds of medicines include but not limited to antibodies, affinity ligand-bioactive agent conjugates such as affinity ligand (e.g. antibody, aptamer, small molecule ligand)-drug conjugates (here the term drug means molecule having bio activity, which can produce certain biological effect to the target, e.g. toxins, enzyme inhibitors and etc, it is not necessary that the drug can be used alone as a medicine), antibody-bioactive molecule conjugates such as antibody-drug conjugates and virus entry inhibitors. Other medicines take effect by bind with the internal receptor of pathogens or human cells. Therefore similar to the method described in the previous applications, a blood purification treatment can be performed to remove the circulating antigens/pathogens/cells having this surface maker or their released surface marker (receptor) and other substance (or the released target receptor if the target receptor is inside the pathogen/cell) in the blood that can bind with the medicine with high affinity before these types of medicine is given to the patient. This will minimize the side effect such as those caused by generating potential harmful immune complex or binding complex, reduce the dosage for the medicine and increase the medicine efficacy. One method is to pass the blood or plasma through solid phase coated with medicine or part of the medicine or its mimic or functional similar molecule that can bind with the same substance to be removed in the extracorporeally circulating treatment. Other methods such as less selective plasmapheresis, apheresis, hemofiltration et ac can also be used as long as the blood part containing these circulating antigens/pathogens/cells or released receptor can be removed. Without removing these circulating antigens/pathogens/cells/released target receptors, the medicine will bind with them to form a binding complex (e.g. an antibody-antigen immune complex if the medicine contains an antibody part) which could be harmful. The medicine can also bind with the circulating soluble antigen molecules (e.g. soluble gp120 in the blood of HIV patient) or other molecules in the blood having high affinity to the medicine, to compete with the medicine binding with its desired target (e.g. the pathogens/cells not in the blood) to reduce the medicine efficacy. If they are removed, the medicine will be more potent because the amount of target accessible medicine is higher, and sometimes less medicine can be used to reduce the side effect. Even if the desired target (e.g. pathogens/cells) is in the blood, removing significant amount them from blood before the patient is given the medicine is also beneficial because the medicine is more effective in treat the residual target and sometimes less medicine can be used to reduce side effect. Preferably the medicine is given to the patient before significant amount of circulating antigens/pathogens/cells/released surface marker (receptor)/released internal receptor is reproduced in the blood after the blood purification. It needs to be pointed out that the medicine suitable for the current invention is not limited to medicines that bind with the surface receptor of the target. It can also be a medicine that binds with the internal receptor (e.g. enzymes, DNA) of the target cell/pathogens. Because the target cell/pathogens can secrete said receptor or release said receptor when they are lysed, the blood will also contains abundant of these receptors, which are not desired target for the medicine's therapeutic efficacy. Removing them from blood before the medicine is given using blood purification will increase the efficacy and safety of the medicine. For example, tumor will release their surface marker or internal receptor into the blood especially when their cells are killed (e.g. apoptosis or under chemo therapy or radio therapy), removing them before giving the corresponding medicine targeting said marker or receptor will increase the treatment efficacy of said medicine, especially during or after the tumor cell killing chemo/radio therapy. Furthermore, sometimes the human or pathogen also produce affinity molecule (e.g. antibodies, receptors) for the biding target of the medicine. Removing these affinity molecules using blood purification before giving the medicine will also increase the efficacy and safety of the medicine. A column filled with solid phase support coated with the binding target of the medicine can be used in blood purification to remove these affinity molecules. For example, before giving patient an HIV drug targeting gp120, one can use a column filled with both solid phase support coated with gp-120 and solid phase support coated with antibody against gp-120 for blood purification.

For example, antibody-drug conjugates (ADCs) are a type of targeted therapy, used for many diseases including cancer. They often consist of an antibody (or antibody fragment such as a single-chain variable fragment linked to a payload drug (often cytotoxic). One can use blood purification to remove the antigen in the blood before the antibody-drug conjugates is given. One can use blood purification to remove the endogenous antibody against this antigen in the blood before the antibody-drug conjugates is given. Furthermore, the blood purification can also be performed after ADCs is given to remove the resulting immune complex in the blood. In one example, Brentuximab vedotin is an antibody-drug conjugate approved to treat anaplastic large cell lymphoma (ALCL) and Hodgkin lymphoma. The compound consists of the chimeric monoclonal antibody Brentuximab (which targets the cell-membrane protein CD30) linked to antimitotic agent monomethyl auristatin E. The patient is first treated with blood purification to remove the soluble CD30 and cells expressing CD 30 in the blood (e.g. the extracorporeally circulating blood of a patient passes through a CD 30 removal column such as a column filled with 100 ml 150 um diameter CNBr-activated Sepharose™ 4B bead coupled with Brentuximab or 100 ml 300 um diameter sephadex beads coupled with Brentuximab, at a flow rate of 150 ml/min for 2 h). Alternatively, the patient can be treated with blood cell separator (apheresis) to remove most of the white blood cells in which the cells expressing CD 30 is inside. Furthermore, CD30 can also be coated on the beads and 50 ml these beads are filled into another column to be used together with the first column during blood purification. Next Brentuximab vedotin is given to the patient for the treatment. Similarly this method can also be used for other antibody based anti tumor medicines (which can be pure antibody instead of drug conjugate) using blood purifier having solid phase support coated with the corresponding medicine or its mimic or functionally similar in terms of binding. In another example, Enfuvirtide is an HIV fusion inhibitor, which binds to gp41 preventing the creation of an entry pore for the capsid of the virus, keeping it out of the cell. A patient with HIV infection is first treated with blood purification to remove the HIV and free gp41 in the blood. The blood of a patient passes through a hollow fiber based plasma separator. The pore size of the membrane of the hollow fiber is 0.5 um, which allow the HIV particle to pass. The plasma part passes through a column filled with 100 ml 100 um diameter Sepharose™ 4B beads coupled with antibody against gp120 and antibody against gp41) and then the treated plasma is combined with the blood cells from the plasma separator to form the cleaned blood. The cleaned blood is sent back to the patient. The blood flow rate is 150 ml/min and the treatment continues for 2 h. Next the patient is given the Enfuvirtide as treatment either using the standard protocol or reduced dose.

Monoclonal antibody therapy is the use of monoclonal antibodies (or mAb) to specifically bind to target cells or proteins. This may then stimulate the patient's immune system to attack those cells. It is possible to create a mAb specific to almost any extracellular/cell surface target, and thus there is a large amount of research and development currently being undergone to create monoclonals for numerous serious diseases (such as rheumatoid arthritis, multiple sclerosis and different types of cancers). There are a number of ways that mAbs can be used for therapy. For example: mAb therapy can be used to destroy malignant tumor cells and prevent tumor growth by blocking specific cell receptors. Variations also exist within this treatment, e.g.

radioimmunotherapy, where a radioactive dose localizes on target cell line, delivering lethal chemical doses to the target. There are many antibody type medicines (e.g. those medicines described in http://en.wikipedia.org/wiki/Monoclonal_antibody_therapy) are suitable for the method of the current invention for many applications (e.g. for cancer and immune disease treatment).

For example, Omalizumab is a humanized IgG1k monoclonal antibody that selectively binds to free human immunoglobulin E (IgE) in the blood and interstitial fluid and to membrane-bound form of IgE (mIgE) on the surface of mIgE-expressing B lymphocytes. Omalizumab does not bind to IgE that is already bound by the high-affinity IgE receptors on the surface of mast cells, basophils, and antigen-presenting dendritic cells. It is approved for allergic asthma treatment. Omalizumab (trade name Xolair, Roche/Genentech and Novartis) is a humanized antibody approved for patients 12 years and older with moderate to severe allergic asthma. However it is only allowed to be used for patient with serum IgE in the range of 30 to about 700 IU/ml . Patient having higher serum IgE level or large body size (therefore high total amount of IgE) requiring high dose Xolair cannot use it due to the dosage limit although they may be the one need it the most. Omalizumab is most effective in patients with smaller body size, lower IgE levels, and frequent hospitalizations in spite of aggressive multidrug asthma therapy. Using high dose of Xolair will also increase the chance of side effect. The current invention disclose a method to allow those patient previously cannot use Xolair to be able to use Xolair and a method to reduce the side effect of Xolair by removing serum IgE (and IgE-bearing cells from peripheral blood if whole blood perfusion is used) from their blood to reduce the serum IgE level prior giving acceptable amount of Xolair to these patient using hemopurification treatment (extracorporeal depletion of IgE and IgE-bearing cells), therefore allow the use of lower dose of Xolair to be effective and safe.

The method comprising the following steps: testing the patient's blood IgE level, calculating the amount of Xolair needed using the known dose formula (e.g. Dose: 0.016 mg×body weight (kg)×IgE level (IU/mL)), if dose is too high (e.g. >allowed dose, for example, the current dose upper level is 750 mg per month), a hemopurification treatment is performed to the patient to reduce the IgE level, next the IgE level is tested again and a reduced dose of Xolair is given to the patient accordingly. If no IgE baring cells are removed, preferably the dose should be enough to neutralize >90% of the serum IgE and membrane-bound form of IgE (mIgE) on the surface of mIgE-expressing B lymphocytes. Even when the original dose is not too high, a hemopurification treatment can still be performed to the patient to reduce the IgE level and then the patient is given the drug (either reduced dose or original dose) to further increase the treatment efficacy. If reduced dose is used, the treatment cost is also reduced.

Alternatively, if a patient suffers from the side effect of Xolair, a hemopurification treatment can be performed to the patient to reduce the IgE level, next the IgE level is tested again and a recued dose of Xolair is given to the patient accordingly (e.g. calculated from the above formula). Studied indicated that urticaria developed in 8 (7.5%) of 106 patients in the high-dose group, 6 (5.7%) of 106 patients in the low-dose group, and 3 (2.9%) of 105 patients in the placebo group. Reducing the dose can reduce the rate and severity of side effect.

Preferably the drug should be given before the IgE level rise significantly again (e.g. rise more than 20%) after the hemopurification, in most case giving the drug within 3 days after the hemopurification will be suitable. This method can also be used for other drugs that bind with IgE.

As those described throughout the current application and the U.S. patent application Ser. No. 13/444,201, the hemopurification treatment to remove IgE and possibly IgE bound cells in the blood involves passing extracorporeal circulating blood or plasma through a hemopurifier, which contains a solid phase adsorbent that has affinity to the IgE. The solid phase adsorbent (e.g. column, filter, fiber, membrane, and particle) is coated with affinity molecules that can selectively bind with IgE. In one example, the patient is first treated with hemopurification to remove the IgE in the blood (e.g. the extracorporeally circulating whole blood of a patient or the plasma of the patient from a plasma separator passes through a IgE removal column such as a column filled with 100 ml 150 um diameter CNBr-activated Sepharose™ 4B bead coupled with Omalizumab or 100 ml 300 um diameter poly acrylic beads coupled with Omalizumab, at a flow rate of 150 ml/min for 2 h). Alternatively, the patient can be treated with plasmapheresis or the like to remove most of the antibody including IgE. Next suitable amount of Omalizumab is given to the patient for the treatment based on the current IgE level of the patient within 3 days after the hemopurification. In some cases, the IgE test can be performed after 1 or 2 days to get a stabilized IgE count.

In another example, the blood of a patient passes through a hollow fiber based plasma separator. The pore size of the membrane of the hollow fiber is 0.3 um. The plasma part passes through a column filled with 100 ml 100 um diameter silica beads coupled with Omalizumab or other antibody against IgE or other affinity ligand for IgE, and then the treated plasma is combined with the blood cells from the plasma separator to form the cleaned blood. The cleaned blood is sent back to the patient. The blood flow rate is 100 ml/min and the treatment continues for 2 h. Next the patient is given the Omalizumab as treatment either using the original dose before the treatment or reduced dose based on the IgE level after the treatment. The blood purification treatment can also be performed without giving Omalizumab to the patient for the indication of Omalizumab.

The plasma separator and the adsorbent can also be integrated in one cartridge same as that used by Aethlon for its lectin based HCV removal ADAPT™ System cartridge, except that the solid phase adsorbent for IgE removal is coated with antibody against IgE instead of the lectin in the ADAPT™ System cartridge.

The solid phase support in the blood purifier can also be coated with other antibody against IgE instead of Omalizumab as long as this antibody can still selectively bind with IgE. Omalizumab inhibits the binding of IgE to the high-affinity IgE receptor FccRI by binding to an epitope on IgE that overlaps with the site to which FccRI binds. This feature is critical to omalizumab's pharmacological effects because a typical anti-IgE antibody can cross-link cell surface FcεRI-bound IgE and induce mediator release from basophils and mast cells. This feature is not required for the antibody used for the blood purification to remove IgE especially when only plasma is used to pass the blood purifier. Antibody from other source (e.g. from goat) and targeting other IgE region can also be used instead. However humanized antibody can provide low immunogenicity since there may be leaks of the antibody into the blood during the treatment. Other affinity ligand such as aptamer, small molecules having high affinity to IgE selectively can also be used to be coupled to the solid phase support instead of using antibodies.

Alternatively, the patient can be treated with plasmapheresis or the like to remove most of the antibody including IgE. Next Omalizumab is given to the patient for the treatment. In another example, the blood of a patient passes through a hollow fiber based plasma separator. The pore size of the membrane of the hollow fiber is 0.3 um. The plasma part passes through a column filled with 100 ml 100 um diameter silica beads coupled with Omalizumab or other antibody against IgE or other affinity ligand for IgE) and then the treated plasma is combined with the blood cells from the plasma separator to form the cleaned blood. The cleaned blood is sent back to the patient. The blood flow rate is 100 ml/min and the treatment continues for 2 h. Next the patient is given the Omalizumab as treatment either using the standard protocol or reduced dose. The blood purification treatment can also be performed without giving Omalizumab to the patient for the indication of Omalizumab. The solid phase support in the blood purifier can also be coated with other antibody against IgE instead of Omalizumab as long as this antibody can still selectively bind with IgE. Omalizumab inhibits the binding of IgE to the high-affinity IgE receptor FccRI by binding to an epitope on IgE that overlaps with the site to which FccRI binds. This feature is critical to omalizumab's pharmacological effects because a typical anti-IgE antibody can cross-link cell surface FccRI-bound IgE and induce mediator release from basophils and mast cells. This feature is not required for the antibody used for the blood purification to remove IgE especially when only plasma is used to pass the blood purifier. Antibody from other source and targeting other IgE region (e.g. from goat) can also be used instead.

Belimumab is a human monoclonal antibody that inhibits B-cell activating factor (BAFF). It is approved in the United States, Canada and Europe for treatment of systemic lupus erythematosus (SLE), and is being tested for use in other autoimmune diseases. B-cell activating factor (BAFF) is secreted, sometimes under the influence of interferon-gamma, by a variety of cells during rheumatoid arthritis, Sjögren's syndrome, and certain glioblastomas. Belimumab binds primarily to circulating soluble BAFF, therefore not inducing antibody-dependent cellular cytotoxicity that could be expected from this IgG1-type antibody.

In one example, the patient is first treated with blood purification to remove the BAFF in the blood (e.g. the extracorporeally circulating whole blood of a patient or the plasma of the patient after a plasma separator passes through a BAFF removal column such as a column filled with 100 ml 150 um diameter CNBr-activated Sepharose™ 4B bead coupled with Belimumab or 100 ml 300 um diameter Sephadex beads coupled with Belimumab, at a flow rate of 100 ml/min for 2 h). Alternatively, the patient can be treated with plasmapheresis or other none selective blood purification method to remove most of the BAFF in the blood. Next Belimumab is given to the patient for the treatment. In another example, the blood of a patient passes through a hollow fiber based plasma separator. The pore size of the membrane of the hollow fiber is 0.3 um. The plasma part passes through a column filled with 50 ml 100 um diameter poly styrene beads coupled with Belimumab or other antibody against BAFF or other affinity ligand for BAFF) and then the treated plasma is combined with the blood cells from the plasma separator to form the cleaned blood. The cleaned blood is sent back to the patient. The blood flow rate is 100 ml/min and the treatment continues for 2 h. Next the patient is given the Belimumab as treatment either using the standard protocol or reduced dose. The plasma separator and the solid phase support can also be integrated within one container by placing the solid phase support outside the hollow fiber therefore no additional 1 blood purifier is needed. The blood purifier can separator the plasma from blood by itself so no plasma in and out outlet on it is needed. The device is similar to that described in FIG. 2 of the said application except no plasma out and plasma return path is needed and the BAFF sorbent is used instead of the pathogen sorbent. The blood purification treatment can also be used alone without giving Belimumab to the patient for the indication applied to Belimumab. The solid phase support in the blood purifier can also be coated with other antibody against BAFF instead of Belimumab as long as this antibody can still selectively bind with BAFF. It can also be other type of affinity ligand for BAFF such as aptamer, membrane receptors on B lymphocytes (B cells) for with BAFF (e.g. BCMA (B cell maturation antigen), TACI (transmembrane activator and calcium modulator and cyclophylin ligand interactor), BAFF-R (BAFF receptor), their binding domain or mimic. For example, Atacicept is a recombinant fusion protein built with the extracellular ligand binding portion of TACI; Blisibimod, an inhibitor of both soluble and membrane bound BAFF; BR3-Fc is a recombinant fusion protein built with the extracellular ligand-binding portion of BAFF-R. These affinity ligands or their mimics can also be used instead of Belimumab to coat the solid phase support used in the blood purifier. Other antibody (e.g. from different source, bind with other BAFF region) can also be used instead as long as they can selectively bind with BAFF. Removing BAFF using a high affinity blood purifier for BAFF can also be used alone instead being used in combination with Belimumab for immune diseases resulting from BAFF.

When antibody drug or antibody-drug conjugates are used, preferably the patient is tested for their blood concentration of the target of the antibody, if it is more than 10 ng/ml, a blood purification step is recommended to remove the free target in the blood. Preferably >50% of the free target in the blood needed to be removed. The drug should be given before the free target concentration rise again (e.g. before the concentration rise 50%). Preferably the drug is given immediately after the blood purification in some cases.

The current invention also discloses methods and device to treat cancer patient by removing the microvesicles in the blood. The method uses a double filtration strategy to remove the microvesicles in the patient's blood by extracorporeally circulating the patient's blood through two filters. The first filter separates the plasma from the blood cells. The second filter having a pore size (e.g. 30 nm or 50 nm) smaller than the size of the microvesicles is then used to remove the microvesicles in the plasma by passing the plasma from the previous step through this second filter. Next the blood cells and the purified plasma are returned to the patient. Tumor cells secrete microvesicles. They were estimated to be between 50-200 nanometers in diameter and associated with a variety of immune inhibitory effects. Specifically, it was demonstrated that such microvesicles could not only induce T cell apoptosis, but also block various aspects of T cell signaling, proliferation, cytokine production, and cytotoxicity. Other research identified another type of microvesicularlike structures, which were termed “exosomes”. Originally defined as small 80-200 nanometers in diameter, exosomes were observed initially in maturing reticulocytes. Subsequently it was discovered that exosomes are a potent method of dendritic cell communication with other antigen presenting cells. Exosomes secreted by dendritic cells were observed to contain extremely high levels of MHC I, MHC II, costimulatory molecules, and various adhesion molecules. In addition, dendritic cell exosomes contain antigens that said dendritic cell had previously engulfed. The ability of exosomes to act as “mini-antigen presenting cells” has stimulated cancer researchers to pulse dendritic cells with tumor antigens, collect exosomes secreted by the tumor antigen-pulsed dendritic cell, and use these exosomes for immunotherapy.

The invention described herein teaches methods of removing microvesicular particles, which include but are not limited to exosomes, from the systemic circulation of a subject in need thereof with the goal of reversing antigen-specific and antigen-nonspecific immune suppression. Said microvesicular particles could be generated by host cells that have been reprogrammed by neoplastic tissue, or the neoplastic tissue itself. Compositions of matter, medical devices, and novel utilities of existing medical devices are disclosed.

A method of removing immune suppressive microvesicular particles from the blood a subject in need thereof, said method comprising: a) establishing an extracorporeal circulation system which comprises contacting the whole blood or components thereof with a filter capable of filtrate the immune suppressive microvesicular particles found within said blood or components thereof to remove said immune suppressive microvesicular particles from said whole blood or components thereof; and b) returning said contacted whole blood or components thereof into the original blood, said contacted whole blood or components thereof containing substantially fewer immune suppressive microvesicular particles in comparison to the whole blood or components thereof originally residing in the subject.

Microvesicles secreted by tumor cells have been known since the early 1980s. They were estimated to be between 50-200 nanometers in diameter and associated with a variety of immune inhibitory effects. Specifically, it was demonstrated that such microvesicles could not only induce T cell apoptosis, but also block various aspects of T cell signaling, proliferation, cytokine production, and cytotoxicity. Although much interest arose in said microvesicles, little therapeutic applications developed since they were uncharacterized at a molecular level. Research occurring independently identified another type of microvesicular-like structures, which were termed “exosomes”. Originally defined as small (i.e., 80-200 nanometers in diameter), exosomes were observed initially in maturing reticulocytes. Subsequently it was discovered that exosomes are a potent method of dendritic cell communication with other antigen presenting cells. Exosomes secreted by dendritic cells were observed to contain extremely high levels of MHC I, MHC II, costimulatory molecules, and various adhesion molecules. In addition, dendritic cell exosomes contain antigens that said dendritic cell had previously engulfed. The ability of exosomes to act as “mini-antigen presenting cells” has stimulated cancer researchers to pulse dendritic cells with tumor antigens, collect exosomes secreted by the tumor antigen-pulsed dendritic cell, and use these exosomes for immunotherapy. Such exosomes were seen to be capable of eradicating established tumors when administered in various murine models. The ability of dendritic exosomes to potently prime the immune system brought about the question if exosomes may also possess a tolerance inducing or immune suppressive role. Since it is established that the exosome has a high concentration of tumor antigens, the question arose if whether exosomes may induce an abortive T cell activation process leading to energy. Specifically, it is known that numerous tumor cells express the T cell apoptosis inducing molecule Fas ligand.

In one aspect, the present invention relates to methods of removing microvesicles from the circulation of a subject in need thereof (e.g., cancer patients), thereby de-repressing immune suppression present in said subjects. Accordingly, the present invention teaches the use of various extracorporeal devices and methods of producing extracorporeal devices for use in clearing microvesicle content in subjects in need thereof. Said microvesicles may be elaborated by the tumor itself, or may be generated by non-malignant cells under the influence of tumor soluble or contact dependent interactions. Said microvesicles may be directly suppressing the host immune system through induction of T cell apoptosis, proliferation inhibition, incapacitation, anergy, deviation in cytokine production capability or cleavage of the T cell receptor zeta chain, or alternatively said microvesicles may be indirectly suppressing the immune system through modification of function of other immunological cells such as dendritic cells, NK cells, NKT cells and B cells. Said microvesicles may be suppressing the host antitumor immune response either in an antigen-specific or an antigen-nonspecific manner, or both.

One of the objects of the present invention is to provide an effective and relatively benign treatment for cancer. Another object is to provide an adjuvant, and/or neoadjuvant therapy to be used in conjunction with currently used cancer treatments that require a functional immune response for efficacy. Another object is to provide an adjuvant, and/or neoadjuvant therapy to be used in conjunction with currently used cancer treatments that stimulate the immune response of a subject in need thereof in an antigen-specific manner. Another object is to provide an adjuvant, and/or neoadjuvant therapy to be used in conjunction with currently used cancer treatments that stimulate the immune response of a subject in need thereof in an antigen-nonspecific manner. Another object is to provide improvements in extracorporeal treatment of cancer through selecting the novel target of tumor associated microvesicles.

In one particular embodiment, the invention provides a device for extracorporeal treatment of blood or a blood fraction such as plasma. This device has a plasma separator and a filter that can remove the microvesicles from the resulting plasma, and a blood circulation circuit through which blood cells flow unimpeded. The device may be constructed in several variations that would be clear to one skilled in the art. Specifically, the device may be constructed as a closed system in a manner that no accumulating reservoir is needed and the filter system accumulates the microvesicles, while non-microvesicle matter is allowed to flow back into the blood circulation system and subsequently returned to the patient. Alternatively, the device may use an accumulator reservoir that is attached to the filter circuit and connected in such a manner so that waste fluid is discarded, but volume replenishing fluid is inserted back into the blood circulation system so the substantially microvesicle purified blood that is reintroduced to said patient resembles a hematocrit of significant homology to the blood that was extracted from said patient. In accordance with another embodiment of the present invention, there are provided methods of potentiating the immunologically mediated anticancer response elicited by vaccination to tumor antigens, said methods comprising: a) immunizing a subject in need thereof using a single or combination of tumor antigens; b) removing immunosuppressive microvesicles from the sera of said subject by extracorporeal means; and c) adjusting the amount of removal of immune suppressive microvesicles based on immune stimulation desired.

During the treatment, the blood of the patient first passes through a plasma separator. There are many type of plasma separator suitable for the current application as long as they can separator the blood cell from the plasma while still keeping the microvesicles in the plasma. For example, a hollow fiber based plasma separator with the 0.5 um pore size of the membrane of the hollow fiber can be used, which allow the microvesicles to pass but retaining the blood cells. The resulting plasma then passes through a second filter having pore size smaller than size of microvesicles needed to be removed. It can be a hollow fiber type filter too. In one example, the pore size on the membrane of this second filter is 30 nm. In another example, the pore size on the membrane of the second filter is 50 nm. In a third example, the pore size on the membrane of the second filter is 80 nm.

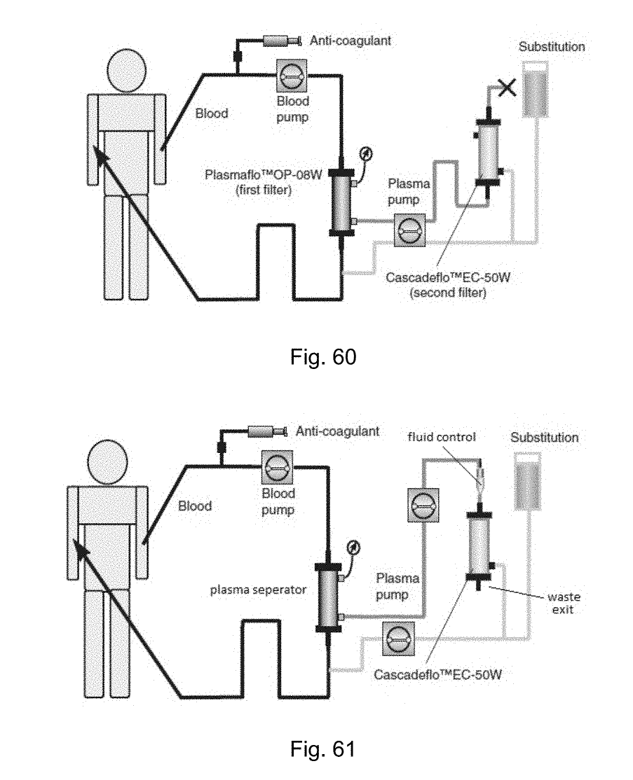

In one example, blood is collected from the peripheral vein for Double filtration plasmapheresis (DFPP, described in FIG. 60), and a Plasmaflo™ OP-08W (Asahi Kasei Medical, Tokyo, Japan) is used to separate the blood into plasma and cell components. The microvesicles are then removed from the separated plasma by a second filter (Cascadeflo™ EC-50W; Asahi Kasei

Medical) with an average pore size of 30 nm. In some cases, for each session, the final volume of treated plasma is 50 mL/kg. The number of sessions and the days when DFPP is given is decided by the physicians, based on the reduced plasma fibrinogen levels during DFPP and patient wishes.

In some embodiments, a fluid control means can be added in the plasma line before the second filter (FIG. 61). The fluid control means is a one way fluid control that only allows the plasma move in one direction without being diffuse back to the plasma separator. It can be a device that generates a separation in the plasma path. The resulting two phases will not contact (or only a little contact) with each other so the substance in second filter will not be able to move back to the plasma separator. Therefore the second filter can have very high concentration of microvesicles but will not diffuse back to the plasma separator. For example, it can be a drip chamber, similar to that used intravenous therapy. The plasma from the plasma separator goes to the drip chamber and falls to the lower liquid phase and then goes to the second filter. It can also be a narrowed path so the plasma travelling speed is increased in that path to prevent diffusion. In one example, the blood of a patient passes through a hollow fiber based plasma separator. The pore size of the membrane of the hollow fiber is 0.3 um. The plasma part passes through a second hollow fiber type filter having a membrane pore size of 50 nm using the system. And then the treated plasma is combined with the blood cells from the plasma separator to form the cleaned blood. The cleaned blood is sent back to the patient. The blood flow rate is 100 ml/min and the treatment continues for 2 h. The plasma in the second filter containing high amount of microvesicles can be drained periodically (e.g. every 30 min) from the waste exit.

Similarly, this kind of fluid control can also be incorporated into other DFPP system for other applications such as removing virus particle from blood by placing it in the path before the second filter and after the plasma separator.

A cartridge filled with adsorbent having affinity to immunosuppressive substance including microvesicles can also be placed in the plasma flow path to further remove these substances either before or after the second filter. Examples of the adsorbent can be found in the literature and those described in U.S. Pat. No. 8,288,172 and it cited reference and those used in HER2osome cartridge from Aethlon biomedical.

Another strategy is to use adsorption column to remove the immune suppressive substance including microvesicles in the blood. Either whole blood or the plasma of patient can be treated with an adsorption column/cartridge. If plasma is being treated, the blood of the patient first passes through a plasma separator to separate the plasma with blood cells. The system and procedure is the same as those described in the DFPP for microvesicles removal except the second filter or both the plasma separator and the second filter is replaced with an adsorption column/cartridge. When the whole blood or plasma passes through the adsorption column, the immune suppressive substance including microvesicles is removed and the clean blood or plasma come out from the exit of the adsorption column to be sent back to the patient.

The adsorption column can be adsorption column filled with charcoal or adsorption resin. The adsorption resin can be either neutral resin or anion exchange resin. Examples of the adsorption column suitable for said application include but not limited to styrene—divinyl benzene copolymer type Adsober Prometh01 Neutral resin filled column (e.g. 100 g resin inside) or Adsober Prometh 02 anion-exchange resin filed column, HA Resin Hemoperfusion cartridge such as HA280 or HA330 cartridge. In one example, during extracorporeal circulation the patient's blood pass through a plasma separator, then plasma part passes through an adsorption column selected from those listed above. And then the treated plasma is combined with the blood cells from the plasma separator to form the cleaned blood. The cleaned blood is sent back to the patient. The blood flow rate is 100 ml/min and the treatment continues for 2 h. Alternatively, no plasma separator is used and the whole blood of the patient passes through the adsorption column and then is returned to the patient. In some embodiments the pore size in the charcoal or adsorption resin is greater than the size of microvesicles to be removed (e.g. preferably >100 nm, more preferably >200 nm).

The adsorption column can also use solid phase support (e.g. resins, particles, fibers) coated with affinity ligand for the immunosuppressive substance including microvesicles. Examples of the affinity ligand can be found in the literature and those described in U.S. Pat. No. 8,288,172 and it cited reference and those used in HER2osome cartridge from Aethlon biomedical. The procedure can be performed in either whole blood perfusion (whole blood pass through the column without prior separating the plasma from blood cells) or plasma hemopurification format.

In another aspect of the current inventions, solid phase support adsorbent with auto antigen coated on the surface can be used in hemopurification to remove the autoimmune T cell or B cell from the patient's blood to treat their auto immune disease, similar to remove the CTC from the patient to treat cancer (e.g. particles) described the current and previous patent application. For example a hemopurifier with adsorbent coated with insulin and/or bata cell surface antigen can be used to remove auto immune T cell/B cell clones to treat diabetes. One can also separate the Lymphocyte from the blood with blood cell separator/leukapheresis and then pass the separated lymphocyte through an affinity column (surface coated with auto antigen) or mix with magnetic particles (surface coated with auto antigen) to remove the autoimmune T cell or B cells and then return the blood/lymphocyte back to the patient. The procedure is similar to the CTC removal described in the current and previous application except the target is B cell or T cells having affinity to certain auto antigens. The current invention discloses the method of T cell and B cell removal with hemopurification to treat the diseases caused by these T cell and/or B cell clones. The U.S. patent application Ser. No. 13/444,201 by the inventor of the current application disclose hemopurification method, device and reagent to remove auto antibody from blood of the patient using hemopurification cartridge containing affinity matrix coated with antigen specific to the auto antibody. The said hemopurification method, device and reagent can be further applied to the whole blood of the patient to remove the T cell and B cell in the blood that are specific to the coated antigen, therefore to treat the immune disease caused by these T cell/B cell clones in the patient. For example, the previous patent application described method, device and reagent to remove CTC from blood using affinity matrix coated with antibody against CTC, when the affinity matrix is coated with pancreatic islet antigen instead, the corresponding method, device and reagent can be used to remove circulating T cells against pancreatic islet therefore to treat diabetes. In another example, the affinity matrix is coated with double strand DNA (e.g. those described in the current invention to conjugate with toxin or alpha-gal), the resulting hemopurification method and device can be used to remove auto immune B cells against DNA, therefore can be used to treat lupus. The antigen can be either B cell antigen or T cell antigen (MHC-peptide complex such as those used for MHC tetramer technology, the MHC and the peptide can be covalently conjugated).

Another aspect of the current invention relates to a method for reducing the viral load by removal of viruses or its fragments or its components or virus infected cell thereof from the blood by extracorporeally circulating blood through solid phase immobilized with affinity molecules having affinity for viral components. Passage of the fluid through the solid phase causes the viral particles and/or virus infected cell to bind to the affinity molecules thereby reducing the viral load in the effluent. Similarly, other pathogens such as bacteria and parasite (e.g. malaria when the red blood cell is broken) can also be removed using with solid phase having affinity molecules with affinity for their components if these pathogens are in the blood.

The solid phase support for blood purification could be a column, a membrane, a fiber, a particle, or any other appropriate surface, which contains appropriate surface properties (including the surface of inside the porous structure) either for direct coupling of the affinity molecules or for coupling after modification or for surface derivatization/modification. If the solid support is porous, its inside can also be used to present the binding affinity molecules.

The current invention also discloses novel absorbents for hemopurification. The solid support of the absorbent is coated with human mannose-binding protein or borate functional group on the surface or borate polymer type synthetic lectins (e.g. benzoboroxole polymer, described in Mol Pharm. 2011 Dec. 5; 8(6): 2465-2475). These absorbent have affinity to sugar rich bio molecules/bio particles/pathogens; therefore can be used to remove virus, bacterial, cells, cytokines, endotoxins, cytokines and immunosuppressive substance including microvesicles from plasma or whole blood, therefore to treat the corresponding diseases. In one embodiment, the blood is withdrawn from the patient and extracorporeal circulating is established. The blood passes through a plasma separator at the flow rate of 200 ml/min. The separated plasma goes into and passes through hemopurification cartridge. The cartridge is a column containing 100 ml adsorbent particle (e.g. 100 um diameter Sepharose 4B beads coupled with recombinant human mannose-binding protein or benzoboroxole polymer). The treated plasma then is combined with blood cells from the plasma separator and goes back to the patient. The entire treatment takes 2 hours.

The current invention also disclose methods to treat sepsis and cytokine storm, autoimmune disease, cancer, fatigue/low appetite (e.g. cancer associated) by removing one or more substances selected from soluble IL-6 receptor-IL-6 complex, soluble IL-6 receptor, IL-6, TNF and TNF receptor in the blood using hemopurification by passing blood or plasma through a cartridge containing one or more solid phase support immobilized affinity ligand (e.g. antibody and aptamer) selected from gp130 or its mimics, antibody against soluble IL-6 receptor-IL-6 complex, antibody against IL-6 receptor (e.g. tocilizumab), antibody against soluble IL-6 receptor, antibody against TNF, antibody against soluble TNF receptor, antibody against IL-6 (e.g. Siltuximab) or aptamers against them, antibody against endotoxin(e.g. Centoxin), affinity ligand for endotoxin(e.g. Polylysine such as ε-Polylysine (ε-poly-L-lysine, EPL)), IL-6 or IL-6 mimic or IL-6 fragment that can bind with soluble IL-6 receptor (to remove soluble IL-6 receptor and/or gp-130) during extracorporeally circulating blood. The current invention also discloses new hemopurification absorbent coated with one or more above affinity ligands to treat sepsis and cytokine storm, IL-6 associated diseases, autoimmune disease, cancer, fatigue/low appetite (e.g. cancer associated). In one example, coupling of antibody or gp130 to the absorbent particle can be performed as follows: 20 mg of particles having surface amine groups (e.g. the 0.2-0.5 mm diameter crosslinked dextran particle such as Sephadex beads or Sepharose 4B or glass beads derivatized to have amine group) are washed three times with 0.1 M MES, pH 5.0 and again three times with deionized water. The particle wet cake is suspended in 0.5 mL of protein (e.g. GP130 or its dimer described in Eur. J. Biochem. 268, 160, 2001 and U.S. patent application Ser. No. 12/026,476; or BMS-945429 a humanized monoclonal antibody against interleukin-6) at 20 mg/mL in deionized water, followed by an addition of 0.5 mL of 20 mg/mL carbodiimide [1-Ethyl-3-(3-dimethyl-aminopropyl)-carbodiimide hydrochloride, EDC] in deionized water, which is prepared immediately before use. The pH is then adjusted to 7.5 with 0.1 M NaHCO3 solution. The particles are rotated at room temperature for 2 hours. Another 10 mg of EDC and 10 mg of NHS (N-hydroxysuccinimide) are added to the mix, followed by an overnight rotation at room temperature. The particles are washed 3 times with 10 mM HEPES buffer, pH 7.5, 5 times with deionized water and then suspended in 1.0 mL of deionized water. The reagent is now ready to be packed in a column for use as absorbent for hemopurification. In one embodiment, the blood is withdrawn from the patient and extracorporeal circulating is established. The blood passes through a plasma separator at the flow rate of 200 ml/min. The separated plasma goes into and passes through hemopurification cartridge. The cartridge is a column containing 50 g adsorbent particle described above. The treated plasma then is combined with blood cells from the plasma separator and goes back to the patient. The entire treatment takes 2 hours. Alternatively, the whole blood without plasma separation can be used to pass the hemopurification cartridge to perform the treatment. To treat sepsis patient, preferably the adsorbent is coated with both affinity ligand for IL-6 (e.g. antibody against IL-6) and affinity ligand for endotoxin (e.g. ε-Polylysine or antibody against endotoxin) and antibody against IL-6 receptor. For example, these types of affinity ligand can be coated on the same Cellufine particles (e.g. prepared using 100 g Cellufine formyl) or the two types of Cellufine particles (e.g. prepared using 50 g Cellufine formyl each) one coated with affinity ligand for endotoxin and another coated with affinity ligand for IL-6 can be mixed together and then packed in one hemopurifier. Furthermore, the adsorbent that can bind with pathogens can also be added to the hemopurification cartridge. Suitable adsorbent for virus and bacterial removal include ε-Polylysine cpated particle, strong cation exchange resin and those solid support having strong negative charged groups or coated with strong negative charged groups described in the previous application by the inventor for virus removal and bacterial removal. ε-Polylysine can kill bacteria, therefore it can be coated to the surface of medical device (e.g. tubing, catheters) to inhibit bacterial growth. For example, the surface of medical device can be derivatized to have —COOH or aldehyde group, then the ε-Polylysine can be coupled covalently to these groups with well known chemistry.

The current invention also disclose method and reagents to treat IL-6 associated diseases (e.g. those involving in IL-6-gp130 signaling, J Clin Invest. 2011 September; 121, 9: 3375-83). The method involves applying administrate (e.g. inject) ligand such as antibody or aptamer to the patient to treat these conditions. This new method to treat disease including sepsis, autoimmune disease and disease caused by IL-6 by administrating the patient with antibody or affinity ligand that can bind with soluble IL-6 receptor or soluble IL-6 receptor-IL-6 complex to prevent it binding with gp130. Suitable affinity ligand includes gp-130 monomer (can be attached with a Fc, or PEG).

Alternatively antibody targeting soluble IL-6 receptor but not inhibit the IL-6 binding with IL-6 receptor, just inhibit IL-6 receptor bind with gp-130 can be used. Gp-130 also binds with other cytokines so the second strategy can reduce the side effect of using gp-130 based affinity ligand. The antibody does not target the region of IL-6 receptor binding with IL-6. It binds with the region that soluble IL-6 receptor binding with gp-130 or provides a steric hindrance that does not allow the soluble IL-6 receptor-IL-6 complex with the cell surface gp-130 or do not allow the aggregation of gp-130 on the cell surface. The antibody can be developed using these sites (e.g.

the C terminal region of soluble IL-6 receptor) as epitope readily by a skilled in the art or screen the antibody library against IL-6 receptor-IL-6 complex to select the desired antibody. Suitable solid phase matrix for the hemopurification in the current invention includes polysaccharide such as cellulose (e.g. Cellufine), agarose, dextran, chitin or chitosan as well as those solid phase support described in the previous application. They can be made as sphere shape.

When the virus infect cell, the cell will present certain viral component (e.g. viral antigen) on the cell surface. So the solid phase support coupled with affinity ligand for virus (preferably the viral antigen on the infected cell surface) will also bind with the cell infected with virus besides the virus. Therefore therapeutical effect to treat viral infection can also be achieved by removing the virus harboring cells from the blood.

In some embodiments, the blood passes through hollow fibers within a cartridge, wherein affinity molecules for virus are immobilized within a porous wall portion of the hollow fiber membrane. Examples of the virus include HIV-1, HBV and HCV. Examples of affinity molecules are antibodies, aptamer, lectin or virus entry inhibitors for these viruses. The affinity molecules can also be attached to a solid matrix and be placed within the blood purification cartridge but outside the porous exterior portion of the hollow fiber. A means that can help the liquid outside the hollow fiber moving (such as pump or stirring device) can be applied to the liquid to increase the diffusing rate. One example of the solid matrix is sepharose. Examples of the hollow fiber membrane can be found in U.S. Pat. No. 6,528,057 and U.S. Pat. No. 7,226,429. The blood purification devices and protocols can also be readily adopted from these patents and other blood purification references. The affinity molecules can also be attached to a solid phase matrix and be placed within the blood purification cartridge and the blood passes through the matrix directly without using hollow fiber. Means that can inactivate the virus such as UV, ultrasound, radiation, heat, microwave and light can also be applied to cartridge or the solid phase within to inactivate the virus inside.

In one example of the method of the present invention, blood is withdrawn from a patient and contacted with the ultra filtration membrane having affinity molecules. In some preferred embodiments, the blood is separated into its plasma and cellular components. The plasma is then contacted with the affinity molecules specific for the virus (or other pathogen) or their surface protein, to remove the virus or components thereof. Following removal of virion (or other pathogen) and/or free nucleic acid, the plasma can then be recombined with the cellular components and returned to the patient. Alternatively, the cellular components may be returned to the patient separately.

Means that can kill the virus or other pathogen can also be applied to the solid phase or the plasma part only. For example, low temperature (e.g. −10 degree) or high temperature (e.g. 40˜60 degree) can be applied to the solid phase support (e.g. the column, filters, tubes, fibers and membrane) or the filter or the separated plasma part. Light (UV or visible light or IR), microwave or radiation can also be applied. Preferably, the means to inactivate pathogen has some selectivity to pathogens over the normal plasma component. For example, if UV is used as means to inactivate pathogens, in some applications the preferred wavelength is the wavelength at which the nucleic acid has high absorption but protein has lower absorption, e.g. 260 nm. Because the captured virus or CTC will stay longer/trap in the solid phase/filter, they will be cool/heat/light or radiation treated much longer time, by carefully control the intensity of the treatment, the virus/CTC will be killed but the healthy cells/plasma component will still be alive/active because they pass through the solid phase/filter quickly. The flow speed, treatment intensity (e.g. temperature, light or radiation intensity) can be adjusted so that only the cells/pathogens stay on the solid phase for a long time will be killed. So even if the virus or other pathogens including CTC are released from the solid phase to the blood they still cannot cause new infection. One method to keep the virus stay longer in the inactivating device is to fill the cartridge of the inactivating device with solid phase support particle having many pore/cavity. The size of the pore/cavity is bigger than the size of the virus but smaller than the blood cell. So when the whole blood pass through the virus will be trapped inside the solid phase and take long time to get out but blood cells will flow away quickly. This mechanism is similar to that of the size exclusion chromatography. Therefore the virus can be treated longer to be inactivated. If photon such as IR, visible light or UV is used to kill the virus, photoactive agents (e.g. those used in photochemical pathogen inactivation for treating blood products) such as phenothiazine dyes, methylene blue, vitamin B2, psoralen(e.g. 8-MOP, AMT), agents used in photodynamic therapy such as photosensitizer can also be added to the blood to increase the virus/pathogen/infected cell inactivating efficacy.

These agents can also be coupled with affinity ligand for the pathogen (e.g. CTC, virus) to increase their selectivity. They can be added to the whole blood or the plasma part or coated on to the solid phase support. These agents (affinity ligand coupled with photo active agent or other cell inactivating agent) can also be coated to the solid phase support such as the surface of the particles or surface of the hollow fiber, therefore it will inactivate/kill the bound pathogens (e.g. virus or CTC) when the solid phase support is being photo irradiated. The affinity ligand and photo active agent can also be co immobilized on the solid phase support instead of conjugating them together, for example by coating the mixture of them to the solid phase support. Besides the photo active regent, other virus/CTC killing regent (such as cytokines, toxins, cell/virus/bacterial killing reagent) can also be co immobilized on the solid phase support with the affinity ligand; or conjugated to the affinity ligand and then the conjugate is immobilized on the solid phase support. Because the virus/CTC killing regent are close to the affinity ligand captured CTC/virus on the solid phase support, the pathogens will be killed.

Examples of toxin/cell inhibitor/inactivator include but not limited to any agent that can kill the cell or inhibit the cell's normal or specific function (e.g. producing certain molecules such as protein (e.g. antibody), replication, differentiation, growth, developing into mature cell or other type of cell). They could be radioactive isotope, proteins, small molecules, siRNA, antisense molecules, enzymes and etc. Examples of them include NK cytotoxic factor, TNF such as TNF-α and TNF-β(LT), perforin, granzyme, cell apoptosis inducers, free radical generating agent, cell membrane damaging agent, toxic agent, chemotherapy agent, siRNA or antisense nucleic acid for the cell normal function, cytotoxic agent and etc. Sometimes they can be made to be in precursor type or inactive type and only become active after they bind with target cell or been taken by the target cell, e.g. the antigen-donomycin conjugate described above. Using affinity molecules coupled with cell damaging reagent is widely used in the treatment of tumor. One can readily adopt the method and principle of them for the current invention. If the cell-damaging reagent is effective only inside the cell, it normally involves a mechanism crossing the cell membrane such as endocytosis.

In one example, the cartridge contains a long tube (e.g 2 meters long)/fiber or multiple hollow fibers (tubes) bundle made of polysulfone membrane or other biocompatible martial. Suitable diameter of the tube/fiber can be selected from 100 um to 3000 um. In one example, the total area of the hollow fiber membrane is 2 m² and the pore size of the membrane is 12 um (the pore of the membrane is optional). One end of the cartridge has blood inlet to connect with the blood from artery and the cartridge also has blood outlet to return blood to the vein. The surface of the fiber/tube is coated with affinity ligand coupled with photo active agent (or other cell inactivating agent). Alternatively, affinity ligand and photo active agent (or other cell inactivating agent are co immobilized on the surface but not conjugated together. Optionally, inside the hollow fiber or tube is filled with solid phase CTC (or other pathogens) adsorbent in the shape of particles or fibers (size>the pore size of hollow fiber membrane, for example, particle size is 100 um) having affinity to the CTC (or other pathogens such as virus). When the blood pass through the cartridge, the red blood cell, platelet, plasma and some white blood cell will pass the wall of the hollow fiber/tube and exit from the cartridge from the blood out outlet and then go back to the patient if the membrane of the fiber/tube contains pores allow small size cells to pass. The affinity captured CTC or virus and some white blood cell/plasma will remain in the hollow fiber/tube. The light (e.g., UV, IR or other wavelength that can activate the photo active agent to kill the cells) radiation can be applied to tube/fiber to kill the affinity captured CTC cells or other pathogens.

For example, photosensitizer such as Photofrin or Levulan can be coupled with antibody against CTC or HIV and then be used as exogenous inactivating affinity material to coat the solid phase support. Photofrin or Levulan or nano particle TiO2 coupled with folic acid or virus entry inhibitor can also be used as exogenous material. When the virus infect cell, the cell will present certain viral component (e.g. viral antigen) on the cell surface. So the exogenous material coupled with affinity ligand for virus (preferably the viral antigen on the infected cell surface) will also kill the cell infected with virus besides the virus by selecting the exogenous material that can damage both human cells and virus. Therefore therapeutical effect to treat viral infection can be achieved by kill the virus harboring cells. Another example of the exogenous inactivating affinity material that can be used to coat the solid phase support can be found at “Extracorporeal photo-immunotherapy for circulating tumor cells” PLoS One. 2015 May 26; 10(5):e0127219.

These agents can also be added to the patient or added to the blood/plasma after the blood is taken out. Furthermore, these agents can be removed from the blood/blood component after the pathogen inactivating treatment but before the blood/blood component is returned to the patient to reduce the potential side effect of these agents to the patient. For example, by passing the blood/blood component through a blood purification device filled with adsorbent (e.g. charcoal, adsorption resin) that can absorb these agents or a blood dialyzer. There are many these types of devices and techniques available for blood purification/blood perfusion/blood dialysis to remove drugs in the blood. One can readily adopt them for the current application. For example, crosslinked agar entrapping attapulgite clay, Pall MB1 filter, Maco Pharma Blueflex filter or LeucoVir MB filter can be used to remove methylene blue in the blood or blood component. If only the plasma part is treated with virus/pathogen killing means (e.g. using a plasma separator to separate the blood cells and the virus containing plasma and then only apply the inactivating means to the plasma part), it may not be always required to remove the virus/pathogen from the plasma using solid phase adsorbent or filter although combining virus killing with solid phase adsorbent or double filtration will increase the therapeutic efficacy. There are many ways to separate plasma from whole blood such as using hollow fiber type plasma separator and many blood component separation devices based on centrifugation. Because many pathogens are in the plasma so treating the plasma only can also reach the pathogen reducing/inactivating effect and reduce the damage to the blood cell. If hollow fiber type plasma separator is used, the pore on the hollow fiber should be big enough to allow pathogen to pass through but not allow most blood cells to pass. In some embodiments, the plasma passes through a filtration device (e.g. a filter) to remove the pathogen inside (e.g. using Double-filtration plasmapheresis) and is also treated with said pathogen inactivating means after or before the filtration. The combination of filtration and pathogen inactivating will result in better therapeutical effect.

The treatment can be repeated periodically until a desired response has been achieved. For example, the treatment can be carried out for 2 hours every 3 days or every week. Thus in some examples, the essential steps of the present invention are (a) contacting the body fluid with the affinity molecule immobilized to an solid phase support (e.g. particles) under conditions that allow the formation of bound complexes of the affinity molecules and their respective target molecules; (b) collecting unbound materials; and (c) reinfusing the unbound materials into the patient.

These methods described in the current invention can also be used to treat other pathogen infection such as bacteria or parasite, as long as they are in the blood. The treatment can be done either in a continuous flow fashion or intermittent flow fashion. For example, the blood is withdrawn continuously and been treated continuously and returned to the patient continuously. In another example, certain volume of blood/blood component is withdrawn and been treated for certain period of time then return to the patient and then the next batch of blood/blood component is withdrawn for treatment. This will allow enough time for the pathogen inactivating. It can also be the combination of continuous flow/intermittent flow. For example, the blood passing through the plasma separator and adsorbent is done continuously but the pathogen inactivating and plasma returning to the patient is done in batch. If the whole blood withdrawing and return is done in an intermittent flow fashion, single needle/catheter in the body can be used for both withdrawing and returning blood in a time slicing fashion by doing them in different time interval.

In some embodiments, the blood or blood component passing through adsorbent is repeated a few times. For example, after the blood or blood component passing through a cartridge filled with adsorbent it is re introduced to the cartridge to allow it pass the adsorbent again before going back to the patient.

Alternatively, the extracorporeal blood circulating is established for a patient with pathogen infection. The whole blood is separated into the blood cells and plasma part. And then pathogen (e.g. virus) containing plasma is treated with physical means (e.g. UV, sonication, radiation, heat, microwave or light) to inactivate the pathogens inside or chemical means (e.g. addition of suitable amount of ozone effective to kill the pathogen into the plasma) to inactivate the pathogens inside. Then the blood cells and the treated plasma are returned to the patient with or without passing through an affinity adsorbent for pathogens. This strategy can also be coupled with the double filtration plasmapheresis to further remove the virus in the pathogen inactivated plasma.

In one example, the extracorporeal blood circulating is established for a patient with HCV infection. The blood passes through a plasma separator at the flow rate of 200 ml/min. The separated plasma goes into and passes through a flat UV transparent container (e.g. an inner size 10×10×1 cm quartz box). The box is irradiated with UV light of 253 nm at the intensity of 60 uW/cm². The plasma travel from one end of the box (plasma inlet) to another end of the box (plasma outlet) in 30 seconds continuously. The treated plasma then is combined with blood cells from the plasma separator and goes back to the patient. The entire treatment takes 2 hours. If desire, the treatment can be repeated several times, e.g. once every 3 days. After the plasma is treated with UV radiation at the above intensity and wavelength, more than 95% HCV virus in the plasma can be inactivated based on the result from virus culture test. Other radiation intensity, wavelength and flow rate and time can also be applied, e.g. 220˜280 nm UV, 30 uW˜3000 uW/cm², 20 seconds to 120 seconds radiation time (the plasma stay time in the radiation path, which is determined by flow rate, shape and size of the radiation path, e.g. the said quartz box). The parameter selected need to provide high pathogen inactivation rate yet low normal plasma protein inactivation rate. For different pathogen, these parameters can be determined experimentally. During the treatment, photoactive agents (e.g. those used in photochemical pathogen inactivation for treating blood products) such as phenothiazine dyes, methylene blue, vitamin B2, S59, psoralen(e.g. 8-MOP, AMT), agents used in photodynamic therapy such as photosensitizer can also be added to the blood or plasma to increase the virus/pathogen/infected cell inactivating efficacy. They can be added either to the plasma directly before the radiation or into the whole blood outside the patient or given to the patient orally or by injection. They can also be coupled with affinity ligand for the pathogens to increase their specificity. The amount added need to be sufficient to inactivate the pathogens under the applied radiation. For example, vitamin B2 can be added to the plasma to reach the concentration of 100 uM and the radiation intensity is 1 mW/cm² at the wavelength of 260 nm-370 nm or 450 nm. A vitamin B2 absorbing cartridge (e.g. a column filled with 100 g of agarose (or gelatin) coated activated charcoal particle) is placed in the downstream of the radiation path to prevent excess vitamin B2 going to the patient. Besides a box shape container, other type of radiation path can also be used such as a spiral tube surrounding a UV lamp. The plasma can either join the blood cell outlet of the plasma separator before going back to the patient or return to the patient directly without combing with the blood cells in which case the plasma separator may not need to have a plasma inlet. Alternatively, heating can be used to inactivating virus instead of UV radiation. For example, the box is placed in a microwave generator and the plasma inside is heated to a temperature of 56 degree. After the plasma is heated at 56 degree, more than 95% HCV virus in the plasma can be inactivated based on the result from virus culture test. Other temperatures can also be used such as those between 50˜70 degree. Alternatively, the plasma is treated with ultrasound instead of with UV or heat. In one example, 1 MHZ 20 W/cm² ultrasound is used to treat the plasma in the container where the plasma travel from one end of the container (plasma inlet) to another end of the container (plasma outlet) in 30 seconds continuously. In another example, a 25 kHZ, 500 W ultrasound generator is placed in the container instead. Furthermore, cartridge filled with HCV adsorbent or a filter with 60 nm pore size can be placed in the downstream of the radiation path to further clean the plasma. Examples of HCV adsorbent include solid phase support coupled with affinity ligand for HCV/their immune complex (e.g. 50 ml 90 um diameter Sepharose 4B beads coupled with a 1:1 molar ratio mixture of Clq and antibody (or lectin) against HCV surface protein).

In another example, the extracorporeal blood circulating is established for a patient with HIV infection. The blood passes through a plasma separator at the flow rate of 100 ml/min. The separated plasma goes into and passes through a flat UV transparent container 5 (e.g. an inner size 10×10×1 cm quartz box). The box is irradiated with UV light of 260 nm at the intensity of 200 uW/cm². The plasma travel from one end of the box (plasma inlet) to another end of the box (plasma outlet) continuously. The treated plasma is then combined with blood cells and goes back to the patient. The entire treatment takes 3 hours. If desire, the treatment can be repeated several times, e.g. once every week. After the plasma is treated with UV radiation at the above intensity and wavelength, more than 95% HIV virus in the plasma can be inactivated based on the result from virus culture test. The plasma separator is filled with HIV adsorbent. The HIV adsorbent contains a mixture of 30 ml of 90 um diameter Sepharose 4B particle coupled with antibody against HIV gp120 and 30 ml of 90 um diameter Sepharose 4B particle coupled with C1q. Alternatively, the plasma is treated with ultrasound instead of with UV. In one example, 1 MHZ 20 W/cm² ultrasound is used to treat the plasma in the container where the plasma travel from one end of the container (plasma inlet) to another end of the container (plasma outlet) in 30 seconds continuously. In another example, a 25 kHZ, 500 W ultrasound generator is placed in the container instead.

The current invention also discloses Antigen-drug conjugate or antigen-alpha gal conjugate for autoimmune disease. The patent application U.S. application Ser. No. 13/444,201 discloses methods to treat autoimmune disease/diseases caused by the production of certain antibody or auto immune T cell against certain foreign antigen or auto antigen. The method involves two steps, in the first step; antibodies or specific antibody or B cells/T cells causing the disease is removed by blood purification procedure. Alternatively, instead of using blood purification, production of antibodies or specific antibody causing the disease is inhibited with drugs. Suitable drug include those can inhibit the production of antibodies such as adrenal corticosteroids, cyclosporin, methotrexate and cellcept. Preferably the dosage is enough to inhibit at least 50% antibody production. The second step is the same as those described in the U.S. Ser. No. 13/444,201 application. When the toxin/cell inhibitor/inactivator-antigen conjugate (e.g. hot suicide antigen) is used to inactivate the antibody production and/or T cells in the second step, the epitope of the antigen need to be selected to be those only bind with specific B cell/T cell/antibody but not other receptors in the body. For example, some diabetes is due to the production of insulin antibody, one can use an insulin epitope-toxin conjugate to inactivate the B cell producing insulin antibody. This epitope need to be selected to only bind with the B cell/T cell/antibody but not the insulin receptor on other human cells.

Many major diseases are caused by auto-antibody (e.g. rheumatoid arthritis and certain diabetes) or bad antibody (e.g. allergy, transplant rejection). Current treatment can not cure from the root and often result in serious side effects (e.g. steroid). ADC (antibody-drug conjugate) becomes a promising cancer treatment in recent years. Antigen-drug conjugate strategy can be used for auto antibody induced autoimmune diseases; selectively inactivate the specific antibody producing B cell clone to cure from the source. The principle was described in U.S. patent application Ser. No. 13/444,201 Methods to detect and treat diseases by the inventor of the current application. Among billions of B cell clones, only several B cell clones produce specific antibody against certain antigen; these B cells secret monoclonal antibody and present membrane bound antibody (BCR receptor) highly specific for target antigen. Antigen-drug conjugate will bind with these B cells with high affinity/high specificity and inactivate them. Selectively inactivating these B cell clones will eliminate the production of harmful antibodies for treating many auto-antibody induced diseases, e.g. lupus, recurrent fetal loss, rheumatoid arthritis, type 1 diabetes, deep vein thrombosis, myasthenia gravis and more.

Companion test (ELISA) to be performed to identify patient having auto antibodies specific to the ADC (similar to the HER2 test for Herceptin): reducing off target. Hemopurification (a clinically used treatment method) using affinity column immobilized with antigen to remove abundant circulating auto-antibodies: one time treatment before ADC administration to improve the ADC efficacy/selectivity for B cells. In most cases no need for protein conjugation, peptide epitope or small molecule antigen will be sufficient for ADC construction, simplify the development/manufacture of ADC. Monthly dosing will be sufficient to prevent somatic hypermutation. T cells also present T cell receptor specific to target antigen, inactivating these T cell clones using antigen-drug conjugate may also be used to treat T-cell-mediated autoimmunity in many major diseases.

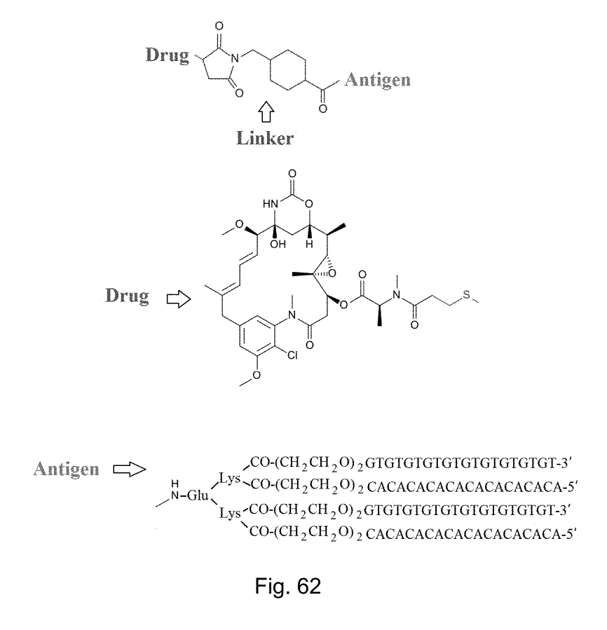

Auto antibody against DNA is a key pathogenic factor in SLE, DNA coated affinity column is clinically used to remove these Ab from patient blood (hemopurification) as an effective SLE treatment. Antigen-drug conjugate can be used for SLE treatment. As shown in FIG. 62, DNA-linker-Mertansine (DNA sequence adopted from Abetimus, linker/toxin adopted from Kadcyla, linker can be optimized for B/T cells) is an example of ADC for SLE treatment. The DNA sequence used are the complex formed with GTGTGTGTGTGTGTGTGTGT (SEQ ID NO: 9) and CACACACACACACACACACA (SEQ ID NO: 10). Single strand DNA Antigen can also be used to inactivate auto antibody generating cells specific to shingle strand DNA. It will selectively inactivate the specific B cell clone producing auto antibody against DNA, treat the disease from the source. It can be prepared easily with solid phase synthesis. Companion test will be performed to increase the efficacy. Patient will be treated with hemopurification to remove the anti-DNA antibody before the first dose ADC administration for better therapeutical index.

In some embodiments preferably the antigen should not bind with the endogenous receptor, for example, insulin fragment that does not bind with insulin receptor but can bind with insulin auto antibody can be used.

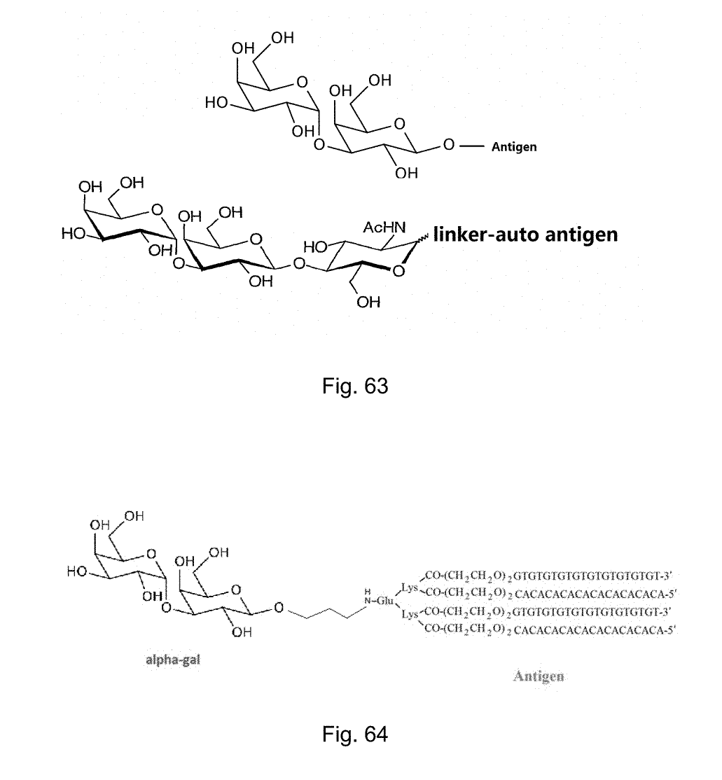

Instead of epitope(antigen)-toxin described in the current application and the previous U.S. application Ser. No. 13/444,201, epitope(antigen)-alpha-gal(e.g. Galactose-alpha-1,3-galactose) can also be used instead, which utilize the endogenous anti gal antibody to inactivate the B cell clone or T cell clone that can selectively bind with the epitope (antigen). The alpha-gal can be readily adopted from U.S. patent application Ser. No. 12/450,384 and other publication. Epitope(antigen)-alpha-gal conjugate design has the formula: alpha-galactosyl-(optional linker)-epitope(antigen), which will allow the T cell/B cell specific to the epitope(antigen) bind with endogenous anti-Gal antibody and therefore be eliminated/inactivated. Examples are shown in FIG. 63.

For example, the antigen can be insulin or insulin fragment that recognized by autoimmune B cell/T cell, or peptide of pancreatic islets recognized by the auto immune T cell in diabetics or the auto antigen of beta cells (e.g. those described in Clin Immunol. 2004 October; 113(1):29-37 and Proc Natl Acad Sci U S A. 2003 Jul. 8; 100(14): 8384-8388). This conjugate will selectively inactive the autoimmune B cell/T cells causing diabetics. For T cell antigen, it can be the MHC-peptide complex form, in which the peptide can be optionally covalently linked with the MHC. An example drug that can selectively inactivate B cells producing auto antibody against DNA is shown in FIG. 64, this drug can be used to treat lupus.

Alternatively, tregitope Peptide-antigen conjugate can be used instead of toxin-antigen conjugate for the same purpose. It will selectively inactivate the autoimmune T cell, therefore treat the corresponding diseases. The carrier system can be used for the above invention as disclosed in U.S. application Ser. No. 13/444,201 by the current inventor. For example, the liposome or microparticle or nano particle can be used. The antigen is immobilized on the surface of the liposome or particles and the effector molecule (e.g. alpha-gal, rhamnose, immuno suppression cytokine, tregitope Peptide, toxin, Si RNA or mi RNA or the like, immune suppressant, antisense molecule) can be either encapsulated inside or co-immobilized on the surface of liposome or particles.

Instead of alpha-gal, other molecule/peptide/protein can also be used to conjugate with a specific antigen to selectively inactivate the specific B cell clone or T cell clone that binds and reacts with the specific antigen. The resulting agent has the general structure:

### Cell Inactivating Molecule-Linker (Optional)-Antigen

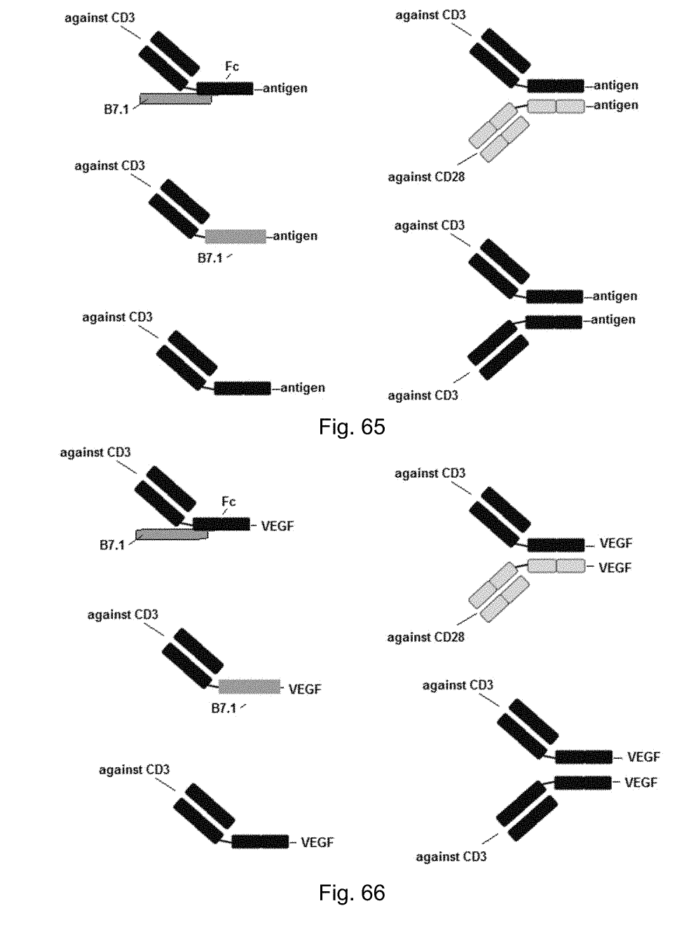

Example of cell inactivating molecule include affinity ligand (e.g. antibody, aptamer) or their combination against immuno cells (e.g. those used in bi specific antibody and triomab for cancer treatment) such as a antibody against a T-lymphocyte antigen like CD3, or a bi specific antibody (or a triomab having Fc) against CD3 and CD28, or a fusion protein of B7 with an antibody (or its fragment) against CD3(examples shown in FIG. 65), antigen that already has immuno response in the body (e.g. alpha-gal, L-rhamnose), B7, super antigen (e.g. staphylococcal enterotoxin A, SEA), cytokines (e.g. immuno cell inactivating cytokines) and those described in the previous patent applications by the inventor and references. For example, L-rhamnose can be linked with a PEG3 by a glycoside bond and the PEG3 is also conjugated with an auto antigen.

SEA is a microbial super-antigen that activates T-lymphocytes and induces production of various cytokines, including interferon-gamma (IFN-gamma), tumor necrosis factor-alpha (TNF-alpha), and cytolytic pore-forming perforin and/or granzyme B secreted by intratumoral CTLs. Example of the SEA gene utilized here can carry the D227A mutation created by Dohlsten's group, which showed a 1000-fold reduction of binding to major histocompatibility complex class (MHC) II in order to decrease systemic toxicity. The protocol of preparing SEA-conjugate can be found at patent applications CN102114239A, CN1629194A and CN101829322A. Besides the co-stimulatory molecules B7.1, other co-stimulatory molecules can also be used such as those selected from other B7 family members including B7.2 (CD86), B7-H1 (PD-Ll), B7-H2 (B7RP-1 or ICOS-L or B7h or GL-50), B7-H3 (B7RP-2), B7-H4 (B7x or B7S1), B7-DC (PD-L2) and etc., and these proteins having amino acid sequence of more than 70% identity of the natural and man-made variants. Co-stimulatory molecules B7.1 (CD80) or other co-stimulatory molecule's role is to stimulate the body's immune response. Furthermore, in addition to B7 family members, other molecules can stimulate T cells can also be used as cell inactivating molecule of the present invention. The protocol described in patent application CN102391377A can be readily adopted for the current invention. For example, the cytokine of the fusion protein in CN102391377A can be replaced with the auto antigen to generate the conjugate of the current application to inactivate the antigen specific B cell and/or T cells.

When the antigen in the conjugate described above and in fig A is replaced with affinity ligand for cancer cells (e.g. antibody against cancer cell or cytokine/peptide/protein having affinity to cancer cells described in paragraph below), it can be used to treat cancer (examples shown in FIG. 66, the VEGF can be VEGF antagonist such as VEGF165b, the VEGF can also be replaced with an antibody or its fragment against cancer cell).

The current invention also discloses methods and agents to treat cancer and kill cancer cells. CN101829322A discloses the use of a cytokine-superantigen fusion protein for preparing a medicament against cancer/tumor, wherein the cytokine is an epidermal growth factor or a vascular endothelial cell growth factor, and the superantigen is the superantigen of staphylococcus aureus enterotoxin A. SEA-conjugates that can be used to treat cancer are also disclosed at patent applications CN102114239A, CN1629194A and CN101829322A. Superantigen fusion protein for anti-cancer therapy and methods for the production is also disclosed at CN1629194A. Patent application CN102391377A discloses a cancer induction and activation of T cells to target the fusion protein and preparation method and use, the protein comprises a peptide with cancer cells and costimulatory molecules B7.1, the cancer cells with a peptide selected Since TGF-α, epidermal growth factor, vascular endothelial growth factor, or gonadotropin-releasing hormone gastrin-releasing peptide, fusion proteins of the invention has a cancer targeting, on the one hand, respectively VEGFR, EGFR, GnRH-R, or GRP-R action, on the other hand with the CD28 receptors expressed on T cells, and CTLA-4 interaction, so it will be targeting T cells targeted to highly expressed VEGFR, EGFR, GnRH-R, GRP-R or around cancer cells, experiments show that the fusion proteins of the invention can inhibit tumor growth and induces apoptosis of cancer cells. The patents listed above utilize B7.1 or super antigen conjugated with a cytokine or peptide or protein that can bind with cancer cell. The current invention disclose a method and agent to treat cancer and kill cancer cells by conjugate the cytokine or peptide or protein used in the above patents (which was conjugated to B7 or super antigen) with alpha-gal or antibody that can bind with immuno cells (such as those used in the bispecific antibody for cancer treatment, e.g. antibody against a T-lymphocyte antigen like CD3). Administering the resulting conjugate to the patient can be used to treat cancer. Several examples of the conjugate are: alpha-gal-linker (optional)-EGF, alpha-gal-linker (optional)-VEGF, alpha-gal-linker(optional)-TGF-α, alpha-gal-GnRH. Preferably the resulting conjugate does not have EGFR/VEGFR agonist activity. When native EGF or VEGFR is used, the conjugate may still have agonist activity. Preferably affinity ligand that can bind with EGFR or VEGFR without activating them, e.g. EGFR or VEGF antagonist, is used to prepare the conjugate. For example, Decorin, VEGF165b, VEGF antagonist in PCT/CA2010/000275 can be used to prepare the conjugate instead of using native VEGF that can activate VEGFR for angiogenesis; they can also be used to conjugate with toxin (such as MMAE, MMAF and DM1) for cancer treatment. These cytokines can be further modified to be peptidase/protease resistant to increase their half life in vivo and a half life modifier such as Fc or fatty acid can be added into the conjugate to increase their half life.

Besides alpha-gal, other antigen that already has T cell immunity or B cell immunity can also be used to replace the alpha-gal in the said conjugate for immuno cell or cancer cell or pathogen inactivation. It can be either endogenous or induced by vaccination using the said antigen. Examples of endogenous antigen include DNP (Dinitrophenyl) and L-rhamnose (e.g. alpha-L-rhamnose). The induced antibody or antigen specific effector T cell can be generated with vaccination. For example, most new born receive the antituberculosis vaccine BCG, the oral poliovirus vaccine (OPV) and the anti-hepatitis B vaccine (HBVac). They will have B cell or T cell immunity against these antigens. One can use the antigen from OPV or BCG or HBV to prepare the conjugate instead of using alpha-gal. The patient can be first tested with his antigen reactivity and select the antigen having strong B cell or T cell immunity to prepare the conjugate and administering this personalized conjugate to the patient to treat his diseases (e.g. cancer or auto immune disease). One can also inject the patient with a vaccine like antigen to allow the patient to develop T cell immunity or B cell immunity against this antigen and then use this antigen to prepare the conjugate for disease treatment. Another example of utilizing native immunity is to use the blood type antigen instead of alpha-gal to build the conjugate: ABO antigen. For example, for patient having Blood type group A, the conjugate can utilize B antigen; for patient having Blood type group B, the conjugate can utilize A antigen; for patient having Blood type group O, the conjugate can utilize either A or B antigen or their combination. In one example, the conjugate of A antigen-double strand DNA can be used to treat blood type B patient having lupus; in another example, the conjugate of B antigen-VEGF165b can be used to treat blood type A patient having cancer.

Compounds described herein can be administered as a pharmaceutical or medicament formulated with a pharmaceutically acceptable carrier. Accordingly, the compounds may be used in the manufacture of a medicament or pharmaceutical composition. Pharmaceutical compositions of the invention may be formulated as solutions or lyophilized powders for parenteral administration. Powders may be reconstituted by addition of a suitable diluent or other pharmaceutically acceptable carrier prior to use. Liquid formulations may be buffered, isotonic, aqueous solutions. Powders also may be sprayed in dry form. Examples of suitable diluents are normal isotonic saline solution, standard 5% dextrose in water, or buffered sodium or ammonium acetate solution. Such formulations are especially suitable for parenteral administration, but may also be used for oral administration or contained in a metered dose inhaler or nebulizer for insufflation. Compounds may be formulated to include other medically useful drugs or biological agents. The compounds also may be administered in conjunction with the administration of other drugs or biological agents useful for the disease or condition to which the invention compounds are directed.

As employed herein, the phrase “an effective amount,” refers to a dose sufficient to provide concentrations high enough to impart a beneficial effect on the recipient thereof. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, and general health of the subject, and like factors well known in the medical arts and sciences. Various general considerations taken into account in determining the “therapeutically effective amount” are known to those of skill in the art and are described.

Dosage levels typically fall in the range of about 0.001 up to 100 mg/kg/day; with levels in the range of about 0.05 up to 10 mg/kg/day are generally applicable. A compound can be administered parenterally, such as intravascularly, intravenously, intraarterially, intramuscularly, subcutaneously, or the like. Administration can also be orally, nasally, rectally, transdermally or inhalationally via an aerosol. The compound may be administered as a bolus, or slowly infused. A therapeutically effective dose can be estimated initially from cell culture assays by determining an IC50. A dose can then be formulated in animal models to achieve a circulating plasma concentration range that includes the IC50 as determined in cell culture. Such information can be used to more accurately determine useful initial doses in humans. Levels of drug in plasma may be measured, for example, by HPLC. The exact formulation, route of administration and dosage can be chosen by the individual physician in view of the patient's condition.

In the current application the “/” mark means either “and” or “or”. Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from text books such as the chemistry textbooks, scientific journal papers and other well known reference sources.

1. A device for treating cytokine storm related syndrome comprising a container which contains and retains adsorbent that can remove soluble IL-6 receptor and IL-6,
wherein said adsorbent comprises affinity ligand for soluble IL-6 receptor and affinity ligand for IL-6 immobilized on solid phase support medium.
wherein said solid phase support medium is sufficiently porous to allow passage of blood cells or plasma therethrough, and
wherein said container has an inlet and an outlet positioned with respect to the adsorbent such that blood or blood component entering the inlet contacts the adsorbent before exiting the container through the outlet.

2. The device of claim 1 wherein the solid phase support medium is in the form of beads.

3. The device of claim 1 wherein the ligand is covalently bound to the beads, and the ligand is selected from antibody against IL-6 and antibody against soluble IL-6 receptor.

4. The device of claim 1 wherein the ligand for soluble IL-6 receptor is GP-130.

5. The device of claim 1 wherein the cytokine storm related syndrome is sepsis.

6. The device of claim 1 further comprising adsorbent that can remove endotoxin.

7. The device of claim 1 wherein in said adsorbent further comprising affinity ligand for endotoxin immobilized on solid phase support medium.

8. A method for treating cytokine storm related syndrome of an animal or human subject comprising removing a portion of blood from the subject, contacting the whole blood or plasma with an adsorbent
wherein said adsorbent comprises ligand immobilized on a solid phase support medium, the ligand consisting of affinity ligand for IL-6 and affinity ligand for soluble IL-6 receptor;
whereby IL-6 and soluble IL-6 receptor are removed from the whole blood or plasma by adsorption to said adsorbent, then returning the blood to the subject.

9. The method of claim 8 wherein the steps of removing, contacting and returning blood are carried out in a continuous flow from and to the subject.

10. The method of claim 8 wherein the affinity ligand is selected from antibody against IL-6, antibody against soluble IL-6 receptor and GP-130.

11. The method of claim 8 wherein the cytokine storm related syndrome is sepsis.

12. The method of claim 8 wherein said adsorbent can further remove endotoxin comprising affinity ligand for endotoxin immobilized on solid phase support medium.


SEQUENCE LISTING (14 sequences)

SEQ ID NO: 1 - protein, 24 aa, Artificial Sequence
  synthetic affinity peptide
NIYNCEPANPSEKNSPSTQYCYSI

SEQ ID NO: 2 - protein, 40 aa, Artificial Sequence
  synthetic affinity peptide 2
LLGPYELWELSHGGSGGSGGSGGSVPLSLYSGGSGGSGGS

SEQ ID NO: 3 - DNA, 20 bp, Artificial Sequence
  synthetic aptamer
cgagaggttggtgtggttgg

SEQ ID NO: 4 - protein, 99 aa, Artificial Sequence
  synthetic peptide linker
GGASEGSDEAEGSEASGEGDGASEGSDEAEGSEASGEGDGASEGSDEAEGSEASGEGDGASEGSDEAEGSEASGEGDGASEGSDEAEGSEASGEGDGGG

SEQ ID NO: 5 - protein, 42 aa, Artificial Sequence
  synthetic peptide linker 2
GGDGSEGSEGEASEGSAEGEGGSEGSEGEASEGSAEGEGDGG

SEQ ID NO: 6 - protein, 37 aa, Artificial Sequence
  synthetic peptide linker
GGSGSGSGTGRGPSWVGGGSGGSARGPSRWGGSGSSG

SEQ ID NO: 7 - protein, 9 aa, Artificial Sequence
  synthetic peptide linker
GESGQGSEG

SEQ ID NO: 8 - protein, 22 aa, Homo sapiens
GLSKGCFGLKLDRIGSMSGLGC

SEQ ID NO: 9 - DNA, 20 bp, Artificial Sequence
  synthetic artificial DNA antigen
gtgtgtgtgtgtgtgtgtgt

SEQ ID NO: 10 - DNA, 20 bp, Artificial Sequence
  synthetic artificial DNA antigen
cacacacacacacacacaca

SEQ ID NO: 11 - protein, 5 aa, Artificial Sequence
  synthetic peptide linker
LPETG

SEQ ID NO: 12 - protein, 6 aa, Artificial Sequence
  synthetic peptide linker
LPETGG

SEQ ID NO: 13 - protein, 5 aa, Artificial Sequence
  synthetic peptide linker
GGGGG

SEQ ID NO: 14 - protein, 9 aa, Artificial Sequence
  synthetic peptide linker
LPETGGGGG